SEQUENCE LISTING (5 sequences)

SEQ ID NO: 1 - DNA, 1203 bp, Homo sapiens
atggcatctctgagtcagctgagtagccacctgaactacacctgtggggcagagaactccacaggtgccagccaggcccgcccacatgcctactatgccctctcctactgcgcgctcatcctggccatcgtcttcggcaatggcctggtgtgcatggctgtgctgaaggagcgggccctgcagactaccaccaactacttagtagtgagcctggctgtggcagacttgctggtggccaccttggtgatgccctgggtggtatacctggaggtgacaggtggagtctggaatttcagccgcatttgctgtgatgtttttgtcaccctggatgtcatgatgtgtacagccagcatccttaatctctgtgccatcagcatagacaggtacactgcagtggtcatgcccgttcactaccagcatggcacgggacagagctcctgtcggcgcgtggccctcatgatcacggccgtctgggtactggcctttgctgtgtcctgccctcttctgtttggctttaataccacaggggaccccactgtctgctccatctccaaccctgattttgtcatctactcttcagtggtgtccttctacctgccctttggagtgactgtccttgtctatgccagaatctatgtggtgctgaaacaaaggagacggaaaaggatcctcactcgacagaacagtcagtgcaacagtgtcaggcctggcttcccccaacaaaccctctctcctgacccggcacatctggagctgaagcgttactacagcatctgccaggacactgccttgggtggaccaggcttccaagaaagaggaggagagttgaaaagagaggagaagactcggaattccctgagtcccaccatagcgcccaagctcagcttagaagttcgaaaactcagcaatggcagattatcgacatctttgaagctggggcccctgcaacctcggggagtgccacttcgggagaagaaggcaacccaaatggtggccattgtgcttggggccttcattgtctgctggctgcccttcttcttgacccatgttctcaatacccactgccagacatgccacgtgtccccagagctttacagtgccacgacatggctgggctacgtgaatagcgccctcaaccctgtgatctataccaccttcaatatcgagttccggaaagccttcctcaagatcctgtcttgctga

SEQ ID NO: 2 - protein, 400 aa, Homo sapiens
MASLSQLSSHLNYTCGAENSTGASQARPHAYYALSYCALILAIVFGNGLVCMAVLKERALQTTTNYLVVSLAVADLLVATLVMPWVVYLEVTGGVWNFSRICCDVFVTLDVMMCTASILNLCAISIDRYTAVVMPVHYQHGTGQSSCRRVALMITAVWVLAFAVSCPLLFGFNTTGDPTVCSISNPDFVIYSSVVSFYLPFGVTVLVYARIYVVLKQRRRKRILTRQNSQCNSVRPGFPQQTLSPDPAHLELKRYYSICQDTALGGPGFQERGGELKREEKTRNSLSPTIAPKLSLEVRKLSNGRLSTSLKLGPLQPRGVPLREKKATQMVAIVLGAFIVCWLPFFLTHVLNTHCQTCHVSPELYSATTWLGYVNSALNPVIYTTFNIEFRKAFLKILSC

SEQ ID NO: 3 - DNA, 2893 bp, Homo sapiens
ggcaccagctcagccccaagccactgctctcccatcccagtccctggaaatccacccacttggcccagctcaccccaactccaacccactgggacccagtctccaggggcctgactgtgggcggcagccactcctgagtgagcaaaggttcctccgcggtgctctcccgtccagagccctgctgatggggaagtccgaaggccccgtggggatggtggagagcgctggccgtgcagggcagaagcgcccggggttcctggagggggggctgctgctgctgctgctgctggtgaccgctgccctggtggccttgggtgtcctctacgccgaccgcagagggaagcagctgccacgccttgctagccggctgtgcttcttacaggaggagaggacctttgtaaaacgaaaaccccgagggatcccagaggcccaagaggtgagcgaggtctgcaccacccctggctgcgtgatagcagctgccaggatcctccagaacatggacccgaccacggaaccgtgtgacgacttctaccagtttgcatgcggaggctggctgcggcgccacgtgatccctgagaccaactcaagatacagcatctttgacgtcctccgcgacgagctggaggtcatcctcaaagcggtgctggagaattcgactgccaaggaccggccggctgtggagaaggccaggacgctgtaccgctcctgcatgaaccagagtgtgatagagaagcgaggctctcagcccctgctggacatcttggaggtggtgggaggctggccggtggcgatggacaggtggaacgagaccgtaggactcgagtgggagctggagcggcagctggcgctgatgaactcacagttcaacaggcgcgtcctcatcgacctcttcatctggaacgacgaccagaactccagccggcacatcatctacatagaccagcccaccttgggcatgccctcccgagagtactacttcaacggcggcagcaaccggaaggtgcgggaagcctacctgcagttcatggtgtcagtggccacgttgctgcgggaggatgcaaacctgcccagggacagctgcctggtgcaggaggacatggtgcaggtgctggagctggagacacagctggccaaggccacggtaccccaggaggagagacacgacgtcatcgccttgtaccaccggatgggactggaggagctgcaaagccagtttggcctgaagggatttaactggactctgttcatacaaactgtgctatcctctgtcaaaatcaagctgctgccagatgaggaagtggtggtctatggcatcccctacctgcagaaccttgaaaacatcatcgacacctactcagccaggaccatacagaactacctggtctggcgcctggtgctggaccgcattggtagcctaagccagagattcaaggacacacgagtgaactaccgcaaggcgctgtttggcacaatggtggaggaggtgcgctggcgtgaatgtgtgggctacgtcaacagcaacatggagaacgccgtgggctccctctacgtcagggaggcgttccctggagacagcaagagcatggtcagagaactcattgacaaggtgcggacagtgtttgtggagacgctggacgagctgggctggatggacgaggagtccaagaagaaggcgcaggagaaggccatgagcatccgggagcagatcgggcaccctgactacatcctggaggagatgaacaggcgcctggacgaggagtactccaatctgaacttctcagaggacctgtactttgagaacagtctgcagaacctcaaggtgggcgcccagcggagcctcaggaagcttcgggaaaaggtggacccaaatctctggatcatcggggcggcggtggtcaatgcgttctactccccaaaccgaaaccagattgtattccctgccgggatcctccagccccccttcttcagcaaggagcagccacaggccttgaactttggaggcattgggatggtgatcgggcacgagatcacgcacggctttgacgacaatggccggaacttcgacaagaatggcaacatgatggattggtggagtaacttctccacccagcacttccgggagcagtcagagtgcatgatctaccagtacggcaactactcctgggacctggcagacgaacagaacgtgaacggattcaacacccttggggaaaacattgctgacaacggaggggtgcggcaagcctataaggcctacctcaagtggatggcagagggtggcaaggaccagcagctgcccggcctggatctcacccatgagcagctcttcttcatcaactatgcccaggtgtggtgcgggtcctaccggcccgagttcgccatccaatccatcaagacagacgtccacagtcccctgaagtacagggtactggggtcgctgcagaacctggccgccttcgcagacacgttccactgtgcccggggcacccccatgcaccccaaggagcgatgccgcgtgtggtagccaaggccctgccgcgctgtgcggcccacgcccacctgctgctcggaggcatctgtgcgaaggtgcagctagcggcgacccagtgtacgtcccgccccggccaaccatgccaagcctgcctgccaggcctctgcgcctggcctagggtgcagccacctgcctgacacccagggatgagcagtgtccagtgcagtacctggaccggagccccctccacagacacccgcggggctcagtgcccccgtcacagctctgtagagacaatcaactgtgtcctgcccaccctccaaggtgcattgtcttccagtatctacagcttcagacttgagctaagtaaatgcttcaaagaaaaaaaaaaaaaaaaaaaaa

SEQ ID NO: 4 - DNA, 2975 bp, Homo sapiens
cagagctcgtttagtgaaccgtcagaattttgtaatacgactcactatagggcggccgcgaattcggcaccagctcagccccaagccactgctctcccatcccagtccctggaaatccacccacttggcccagctcaccccaactccaacccactgggacccagtctccaggggcctgactgtgggcggcagccactcctgagtgagcaaaggttcctccgcggtgctctcccgtccagagccctgctgatggggaagtccgaaggccccgtggggatggtggagagcgctggccgtgcagggcagaagcgcccggggttcctggagggggggctgctgctgctgctgctgctggtgaccgctgccctggtggccttgggtgtcctctacgccgaccgcagagggaagcagctgccacgccttgctagccggctgtgcttcttacaggaggagaggacctttgtaaaacgaaaaccccgagggatcccagaggcccaagaggtgagcgaggtctgcaccacccctggctgcgtgatagcagctgccaggatcctccagaacatggacccgaccacggaaccgtgtgacgacttctaccagtttgcatgcggaggctggctgcggcgccacgtgatccctgagaccaactcaagatacagcatctttgacgtcctccgcgacgagctggaggtcatcctcaaagcggtgctggagaattcgactgccaaggaccggccggctgtggagaaggccaggacgctgtaccgctcctgcatgaaccagagtgtgatagagaagcgaggctctcagcccctgctggacatcttggaggtggtgggaggctggccggtggcgatggacaggtggaacgagaccgtaggactcgagtgggagctggagcggcagctggcgctgatgaactcacagttcaacaggcgcgtcctcatcgacctcttcatctggaacgacgaccagaactccagccggcacatcatctacatagaccagcccaccttgggcatgccctcccgagagtactacttcaacggcggcagcaaccggaaggtgcgggaagcctacctgcagttcatggtgtcagtggccacgttgctgcgggaggatgcaaacctgcccagggacagctgcctggtgcaggaggacatggtgcaggtgctggagctggagacacagctggccaaggccacggtaccccaggaggagagacacgacgtcatcgccttgtaccaccggatgggactggaggagctgcaaagccagtttggcctgaagggatttaactggactctgttcatacaaactgtgctatcctctgtcaaaatcaagctgctgccagatgaggaagtggtggtctatggcatcccctacctgcagaaccttgaaaacatcatcgacacctactcagccaggaccatacagaactacctggtctggcgcctggtgctggaccgcattggtagcctaagccagagattcaaggacacacgagtgaactaccgcaaggcgctgtttggcacaatggtggaggaggtgcgctggcgtgaatgtgtgggctacgtcaacagcaacatggagaacgccgtgggctccctctacgtcagggaggcgttccctggagacagcaagagcatggtcagagaactcattgacaaggtgcggacagtgtttgtggagacgctggacgagctgggctggatggacgaggagtccaagaagaaggcgcaggagaaggccatgagcatccgggagcagatcgggcaccctgactacatcctggaggagatgaacaggcgcctggacgaggagtactccaatctgaacttctcagaggacctgtactttgagaacagtctgcagaacctcaaggtgggcgcccagcggagcctcaggaagcttcgggaaaaggtggacccaaatctctggatcatcggggcggcggtggtcaatgcgttctactccccaaaccgaaaccagattgtattccctgccgggatcctccagccccccttcttcagcaaggagcagccacaggccttgaactttggaggcattgggatggtgatcgggcacgagatcacgcacggctttgacgacaatggccggaacttcgacaagaatggcaacatgatggattggtggagtaacttctccacccagcacttccgggagcagtcagagtgcatgatctaccagtacggcaactactcctgggacctggcagacgaacagaacgtgaacggattcaacacccttggggaaaacattgctgacaacggaggggtgcggcaagcctataaggcctacctcaagtggatggcagagggtggcaaggaccagcagctgcccggcctggatctcacccatgagcagctcttcttcatcaactatgcccaggtgtggtgcgggtcctaccggcccgagttcgccatccaatccatcaagacagacgtccacagtcccctgaagtacagggtactggggtcgctgcagaacctggccgccttcgcagacacgttccactgtgcccggggcacccccatgcaccccaaggagcgatgccgcgtgtggtagccaaggccctgccgcgctgtgcggcccacgcccacctgctgctcggaggcatctgtgcgaaggtgcagctagcggcgacccagtgtacgtcccgccccggccaaccatgccaagcctgcctgccaggcctctgcgcctggcctagggtgcagccacctgcctgacacccagggatgagcagtgtccagtgcagtacctggaccggagccccctccacagacacccgcggggctcagtgcccccgtcacagctctgtagagacaatcaactgtgtcctgcccaccctccaaggtgcattgtcttccagtatctacagcttcagacttgagctaagtaaatgcttcaaagaaaaaaaaaaaaaaaaaaaaactcgactctagattgcg

SEQ ID NO: 5 - protein, 779 aa, Homo sapiens
MGKSEGPVGMVESAGRAGQKRPGFLEGGLLLLLLLVTAALVALGVLYADRRGKQLPRLASRLCFLQEERTFVKRKPRGIPEAQEVSEVCTTPGCVIAAARILQNMDPTTEPCDDFYQFACGGWLRRHVIPETNSRYSIFDVLRDELEVILKAVLENSTAKDRPAVEKARTLYRSCMNQSVIEKRGSQPLLDILEVVGGWPVAMDRWNETVGLEWELERQLALMNSQFNRRVLIDLFIWNDDQNSSRHIIYIDQPTLGMPSREYYFNGGSNRKVREAYLQFMVSVATLLREDANLPRDSCLVQEDMVQVLELETQLAKATVPQEERHDVIALYHRMGLEELQSQFGLKGFNWTLFIQTVLSSVKIKLLPDEEVVVYGIPYLQNLENIIDTYSARTIQNYLVWRLVLDRIGSLSQRFKDTRVNYRKALFGTMVEEVRWRECVGYVNSNMENAVGSLYVREAFPGDSKSMVRELIDKVRTVFVETLDELGWMDEESKKKAQEKAMSIREQIGHPDYILEEMNRRLDEEYSNLNFSEDLYFENSLQNLKVGAQRSLRKLREKVDPNLWIIGAAVVNAFYSPNRNQIVFPAGILQPPFFSKEQPQALNFGGIGMVIGHEITHGFDDNGRNFDKNGNMMDWWSNFSTQHFREQSECMIYQYGNYSWDLADEQNVNGFNTLGENIADNGGVRQAYKAYLKWMAEGGKDQQLPGLDLTHEQLFFINYAQVWCGSYRPEFAIQSIKTDVHSPLKYRVLGSLQNLAAFADTFHCARGTPMHPKERCRVW

(19) United States
(12) Patent Application Publication
(10) Pub. No.: US 2006/0052435 A1
(43) Pub. Date: Mar. 9, 2006

(54) Selective dopamin d3 receptor agonists for the treatment of sexual dysfunction

(76) Inventors: Pieter Hadewijn Van der Graaf, County of Kent (GB); Christopher Peter Wayman, County of Kent (GB); Andrew Douglas Baxter, Bury St. Edmunds (GB); Andrew Simon Cook, County of Kent (GB); Stephen Kwok-Fung Wong, Guilford, CT (US)

Correspondence Address: PFIZER INC., PATENT DEPARTMENT, MS8260-1611, EASTERN POINT ROAD, GROTON, CT, 06340, US

(21) Appl. No.: 10/499,210

(22) PCT Filed: Dec. 10, 2002
(86) PCT No.: PCT/GB02/05595

§ 371 Date: Aug. 26, 2005

(30) Foreign Application Priority Data
Dec. 18, 2001 (GB) 01302199

(51) Int. Cl. A61K 31/405

(52) U.S. Cl. 514/419

(57) ABSTRACT

The use of a composition comprising a selective dopamine D3 receptor agonist, wherein said dopamine D3 receptor agonist is at least about 15-times more functionally selective for a dopamine D3 receptor as compared with a dopamine D2 receptor when measured using the same functional assay, in the preparation of a medicament for the treatment and/or prevention of sexual dysfunction.

### FIELD OF INVENTION

The present invention relates to a compound and a pharmaceutical that is useful for the treatment and/or prevention of sexual dysfunction, for example female sexual dysfunction (FSD), in particular female sexual arousal disorder (FSAD), anorgasmia (inability to achieve orgasm) or desire disorders, such as hypoactive sexual desire disorder (HSDD; lack of interest in sex). Preferably, FSAD with concomitant HSDD is treated or prevented.

The present invention further relates to a method of treatment and/or prevention of FSD, in particular FSAD, anorgasmia or HSDD. Preferably, FSAD with concomitant HSDD is treated or prevented.

The present invention yet further relates to assays to screen for compounds useful in the treatment and/or prevention of FSD, in particular FSAD, anorgasmia or HSDD. Preferably, FSAD with concomitant HSDD is treated or prevented.

The present invention relates to a compound and a pharmaceutical composition for use in the treatment and/or prevention of male sexual dysfunction, in particular male erectile dysfunction (MED). Male sexual dysfunction as referred to herein is meant to include ejaculatory disorders, such as anorgasmia (inability to achieve orgasm) or desire disorders, such as hypoactive sexual desire disorder (HSDD; lack of interest in sex).

The present invention further relates to a method of treatment and/or prevention of male sexual dysfunction, in particular MED.

The present invention also relates to assays to screen for compounds useful in the treatment and/or prevention of male sexual dysfunction, in particular MED.

In one broad aspect, the present invention provides the use of a composition comprising a selective dopamine D3 receptor agonist, wherein said dopamine D3 receptor agonist is at least about 15-times more functionally selective for a dopamine D3 receptor as compared with a dopamine D2 receptor when measured using the same functional assay, in the preparation of a medicament for the treatment and/or prevention of sexual dysfunction.

### BACKGROUND TO THE INVENTION

Dopamine Agonists

Non-selective activation of dopamine receptors within the brain is a clinically proven mechanism to treat male erectile dysfunction (MED). Apomorphine is one such non-selective dopamine agonist, that acts on dopamine receptors within the central nervous system and which is efficacious in the treatment of MED.

Prior to the present invention, it was generally understood that the erectogenic effects and female sexual motivation induced by apomorphine and other non-selective dopamine receptor agonists were mediated by D2 (also termed D₂) dopaminergic receptor activation (see Andersson (2001) Am. Soc. Of Pharmacology and Exp. Therapeutics, Vol 53, No. 3, p 417-450 and Giuliano and Allard (2001) Int. J. Impotence Research, 13, Suppl. 3, S18-S28).

In particular, Chen et al (J. of Urology, July 1999, Vol. 162, 237-242) states that: “an increase in ICP [intracavernosal pressure] to apomorphine was due mainly to activation of the D2 receptor subtype in the PVN [paraventricular nucleus]” (see abstract).

Moreover, further confirmation that prior to the present invention, stimulation of sexual behaviour was thought to be D2 receptor-mediated can be found in Meglasson M. D. et al. (2001) Abstr. Soc. Neurosci., wherein it is stated that:
“ . . . Dopamine receptors in the brain are implicated in sexual behavior.

Earlier findings suggest that the D1 receptor antagonizes the prosexual effects of stimulating D2 receptors (Life Sci 51:1705). This implies that selective D2 agonists may be more effective than a mixed D1/D2 agonist, e.g., apomorphine (APO; Ki: D1, 56 nM vs. D2, 26 nM). We tested this hypothesis using PNU-142774E, a selective D2 receptor agonist (Ki: D2, 7 nM vs. D1>7 uM). Sexual receptivity was measured in ovariectomized, partially hormone-repleted female rats by the lordosis/mounts ratio (L/M) at 15-30 min after dosing with 50 ug/kg PNU-142774E P.O., 50 or 250 g/kg APO I.P., or placebo (time interval matches drugs+/−plasma Cmax). PNU-142774E increased the L/M from 22+/−8% to 65+/−10% (P<0.01). APO did not increase the L/M at the doses tested (%: 10+/−5, 11+/−5, 0+/−0 at 0, 50, and 250 ug/kg, respectively).

These data indicate that a selective D2 agonist is more effective than APO, a drug with mixed D1/D2 receptor agonism. To confirm the relevance of selective D2 receptor agonism to sexual behavior, PNU-142774E was tested in male rhesus monkeys in the presence or absence of sexually-receptive female monkeys. Non-contact sexual behavior was evaluated for 4 hr after a dose of 0, 20, 50, or 125 ug/kg P.O. PNU-142774E produced an increase in sexual behavior (erections, masturbation) at 20 and 50 ug/kg that was enhanced when visual contact with female monkeys was permitted. At 125 ug/kg, sexual behavior was similar to placebo indicating a biphasic dose-response relationship. These findings demonstrate that stimulation of sexual behavior by dopaminergic drugs reflects D2 receptor activation.

Non-selective dopamine agonists, such as apomorphine, 7-hydroxy-DPAT (7-OH-DPAT) and pramipexole, all induce adverse side effects, including nausea, emesis, syncope, hypotension and bradycardia, some of which are a cause for serious concern. In particular the Food and Drug Administration (FDA) in the USA is presently reviewing Uprima® (a.i. apomorphine) following safety concerns due to serious adverse events including syncope, hypotension and bradycardia.

Five dopamine receptors have been cloned. D1-like receptors (D1 and D5; also termed D₁ and D₅) and D2-like receptors (D2, D3 and D4; also termed D₂, D₃ and D₄). By “non-selective” we mean dopamine agonists that display no or only a limited degree of functional selectivity between the different members of the D2-like receptor family, and in particular between D2 and D3 receptors. In WO00/23056, it is suggested that pramipexole is a selective D3 receptor agonist. However, subsequent evaluation determined that functionally pramipexole is only about 9-fold selective for D3 receptors over D2 receptors. This is in agreement with the data shown in Perachon et al. (1999), European Journal of Pharmacology, 366, 293-300. However, yet further evaluation has determined that functionally pramipexole is only as little as about 5-fold selective for D3 receptors over D2 receptors. This is in agreement with other (earlier) work (see Mierau et al. (1995), European Journal of Pharmacology, 290, 29-36). Thus, it is clear that such a compound still possesses potent activity at D2 receptors despite being slightly more active at D3 receptors. Such slightly selective D3 receptor agonists do not fall within the term “selective D3 receptor agonists” as used herein when referring to the present invention, as a selective D3 receptor agonist according to the present invention is selective for a dopamine D3 receptor as compared with a dopamine D2 receptor, which selectivity is, when measured using the same functional assay (i.e. functional agonism assay), at least 3-times the selectivity achieved by the control, slightly D3-preferring (non-selective) compound pramipexole.

Furthermore, with dopamine D3 and D2 receptors, binding data or binding selectivity data has been shown to not always correlate with or reflect functional data or functional selectivity data. For example, compound PNU-95666 ((R)-5,6-dihydro-N,N-dimethyl-4H-imidazo4,5,1-ij)quinolin-5-amine) is a D2 selective compound when binding assays are analysed (U95666A is a high intrinsic activity agonist with selectivity for the D2 dopamine receptor. Lajiness M. E. et al. Abstr Soc Neurosci 1996, 22:1 (217); Synthesis and biological activities of (R)-5,6-dihydro-N,N-dimethyl-4H-imidazo4,5,1-ij)quinolin-5-amine and its metabolites, Heier, R. F. et al., (1997), J. Med. Chem. 40(5) 639-646.), but functionally this compound has the same potency at D2 and D3 receptors. Thus, in prior art disclosures compounds referred to as being selective D3 agonists are often only selective D3 agonists in respect of binding, but are not functionally selective D3 receptor agonists. The term “selective” as used herein in relation to the present invention means “functionally selective”.

Sexual Dysfunction

Sexual dysfunction (SD) is a significant clinical problem which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes self-esteem and disrupts personal relationships thereby inducing personal distress. In the clinic, SD disorders have been divided into female sexual dysfunction (FSD) disorders and male sexual dysfunction (MSD) disorders (Melman et al 1999). FSD is best defined as the difficulty or inability of a woman to find satisfaction in sexual expression. Male sexual dysfunction (MSD) is generally associated with erectile dysfunction, also known as male erectile dysfunction (MED) (Benet et al 1994—Male Erectile dysfunction assessment and treatment options. Comp. Ther. 20: 669-673).

Drug-induced sexual dysfunction can result from therapy with, for example, selective serotonin reuptake inhibitors (SSRis) and other antidepressant therapies (tricyclics and major tranquillizers), anti-hypertensive therapies, and sympatholytic drugs. An example of such a drug-induced sexual dysfunction is anorgasmia (inability to achieve orgasm), which is a type of orgasmic disorder which can occur in both males and females.

The compounds of the invention are particularly beneficial for the prophylaxis and/or treatment of sexual dysfunction in the male (e.g. male erectile dysfunction—MED) and in the female—female sexual dysfunction (FSD), e.g. female sexual arousal disorder (FSAD), anorgasmia or desire disorders, such as hypoactive sexual desire disorder (HSDD; lack of interest in sex). Preferably, in females, FSAD with concomitant HSDD is treated or prevented.

Female Sexual Dysfunction (FSD)

In accordance with the invention, FSD can be defined as the difficulty or inability of a woman to find satisfaction in sexual expression. FSD is a collective term for several diverse female sexual disorders (Leiblum, S. R. (1998)—Definition and classification of female sexual disorders. Int. J. Impotence Res., 10, S104-S106; Berman, J. R., Berman, L. & Goldstein, I. (1999)—Female sexual dysfunction: Incidence, pathophysiology, evaluations and treatment options. Urology, 54, 385-391). The woman may have lack of desire, difficulty with arousal or orgasm, pain with intercourse or a combination of these problems. Several types of disease, medications, injuries or psychological problems can cause FSD. Treatments in development are targeted to treat specific subtypes of FSD, predominantly desire and arousal disorders.

The categories of FSD are best defined by contrasting them to the phases of normal female sexual response: desire, arousal and orgasm (Leiblum, S. R. (1998)—Definition and classification of female sexual disorders. Int. J. Impotence Res., 10, S104-S106). Desire or libido is the drive for sexual expression. Its manifestations often include sexual thoughts either when in the company of an interested partner or when exposed to other erotic stimuli. Arousal is the vascular response to sexual stimulation, an important component of which is genital engorgement and includes increased vaginal lubrication, elongation of the vagina and increased genital sensation/sensitivity. Orgasm is the release of sexual tension that has culminated during arousal.

Hence, FSD occurs when a woman has an inadequate or unsatisfactory response in any of these phases, usually desire, arousal or orgasm. FSD categories include hypoactive sexual desire disorder, sexual arousal disorder, orgasmic disorders and sexual pain disorders. Although the compounds of the invention will improve the genital response to sexual stimulation (as in female sexual arousal disorder), in doing so it may also improve the associated pain, distress and discomfort associated with intercourse and so treat other female sexual disorders.

Thus, in accordance with a preferred aspect of the invention, there is provided use of a compound of the invention in the preparation of a medicament for the treatment or prophylaxis of hypoactive sexual desire disorder, sexual arousal disorder, orgasmic disorder and sexual pain disorder, more preferably for the treatment or prophylaxis of sexual arousal disorder, orgasmic disorder, and sexual pain disorder, and most preferably in the treatment or prophylaxis of sexual arousal disorder.

Hypoactive sexual desire disorder (HSDD) is present if a woman has no or little desire to be sexual, and has no or few sexual thoughts or fantasies. This type of FSD can be caused by low testosterone levels, due either to natural menopause or to surgical menopause. Other causes include illness, medications, fatigue, depression and anxiety. The judgement of deficiency or absence of sexual desire is made by the clinician, taking into account factors that affect functioning, such as age and the context of the persons life.

Female orgasmic disorder (FOD) is considered to be the persistent or recurrent delay in, or absence of, orgasm following a normal sexual excitement phase. Women exhibit wide variability in the type or intensity of stimulation that triggers orgasm. The diagnosis of FOD should be based on the clinician's judgement that the woman's orgasmic capacity is less than would be reasonable for her age, sexual experience, and the adequacy of the sexual stimulation she receives. FOD, especially absence of orgasm, is also termed “anorgasmia”.

Sexual pain disorders (e.g. dyspareunia and vaginismus) are characterised by pain resulting from penetration and may be caused by medications which reduce lubrication, endometriosis, pelvic inflammatory disease, inflammatory bowel disease or urinary tract problems. Dysparenuia is the recurrent or persistent genital pain associated with sexual intercourse. Vaginismus is the recurrent or persistent involuntary spasm of the musculature of the outer third of the vagina that interferes with sexual intercourse.

Female sexual arousal disorder (FSAD) is characterised by inadequate genital response to sexual stimulation. The genitalia do not undergo the engorgement that characterises normal sexual arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants, e.g. selective serotonin re-uptake inhibitors (SSRIs), or antihypertensive agents.

The prevalence of FSD is difficult to gauge because the term covers several types of problem, some of which are difficult to measure, and because the interest in treating FSD is relatively recent. Many women's sexual problems are associated either directly with the female ageing process or with chronic illnesses such as diabetes and hypertension.

Because FSD consists of several subtypes that express symptoms in separate phases of the sexual response cycle, there is not a single therapy. Current treatment of FSD focuses principally on psychological or relationship issues. Treatment of FSD is gradually evolving as more clinical and basic science studies are dedicated to the investigation of this medical problem. Female sexual complaints are not all psychological or pathophysiological, especially for those individuals who may have a component of vasculogenic dysfunction (e.g. FSAD) contributing to the overall female sexual complaint. There are at present no drugs licensed for the treatment of FSD. Empirical drug therapy includes oestrogen administration (topically or as hormone replacement therapy), androgens or mood-altering drugs, such as buspirone or trazodone. These treatment options are often unsatisfactory due to low efficacy or unacceptable side effects.

Since interest is relatively recent in treating FSD pharmacologically, therapy consists of the following: psychological counselling, over-the-counter sexual lubricants, and investigational candidates, including drugs approved for other conditions. These medications consist of hormonal agents, either testosterone or combinations of oestrogen and testosterone, and more recently vascular drugs, that have proved effective in male erectile dysfunction (MED).

As discussed, the compounds of the invention are particularly useful for the prophylaxis and/or treatment of FSD, in particular FSAD, anorgasmia or HSDD.

Hypoactive Sexual Desire Disorder (HSDD)

HSDD is present if a woman has no or little desire to be sexual, and has no or few sexual thoughts or fantasies. This type of FSD can be caused by low testosterone levels, due either to natural menopause or to surgical menopause. Other causes in both pre-menopausal woman (i.e. woman who are pre-menopausal and who have not have hysterectomies) as well as post-menopausal women include illness, medications, fatigue, depression and/or anxiety. Factors having a potential (conscious or sub-conscious) psychological impact such as relationship difficulties or religious factors may be related to the presence of/development of HSDD in females.

Female Sexual Arousal Disorder (FSAD)

The Diagnostic and Statistical Manual (DSM) IV of the American Psychiatric Association defines Female Sexual Arousal Disorder (FSAD) as being:

“ . . . a persistent or recurrent inability to attain or to maintain until completion of the sexual activity adequate lubrication-swelling response of sexual excitement. The disturbance must cause marked distress or interpersonal difficulty. . . . ”.

The arousal response consists of vasocongestion in the pelvis, vaginal lubrication and expansion and swelling of the external genitalia. The disturbance causes marked distress and/or interpersonal difficulty.

FSAD is a highly prevalent sexual disorder affecting pre-, per- and post-menopausal (± hormone replacement therapy (HRT)) women. It is associated with concomitant disorders such as depression, cardiovascular diseases, diabetes and urogenital (UG) disorders.

The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

It has recently been hypothesised that there is a vascular basis for at least a proportion of patients with symptoms of FSAD (Goldstein et al., Int. J. Impot. Res., 10, S84-S90, 1998) with animal data supporting this view (Park et al., Int. J. Impot. Res., 9, 27-37, 1997).

Drug candidates for treating FSAD, which are under investigation for efficacy, are primarily erectile dysfunction therapies that promote circulation to male genitalia. They consist of two types of formulation, oral or sublingual medications (Apomorphine, Phentolamine, phosphodiesterase type 5 (PDE5) inhibitors, e.g. Sildenafil), and prostaglandin (PGE₁) that are injected or administered transurethrally in men and topically to the genitalia in women.

The compounds of the present invention are advantageous by providing a means for restoring a normal sexual arousal response—namely increased genital blood flow leading to vaginal, clitoral and labial engorgement. This will result in increased vaginal lubrication via plasma transudation, increased vaginal compliance and increased genital sensitivity. Hence, the present invention provides a means to restore, or potentiate, the normal sexual arousal response.

The compounds of the present invention are also advantageous by providing a means for restoring (i) a normalised desire and/or (ii) an interest in sex, thereby preventing or treating reduced sexual desire disorders, such as HSDD.

By female genitalia herein we mean: “The genital organs consist of an internal and external group. The internal organs are situated within the pelvis and consist of ovaries, the uterine tubes, uterus and the vagina. The external organs are superficial to the urogenital diaphragm and below the pelvic arch.

They comprise the mons pubis, the labia majora and minora pudendi, the clitoris, the vestibule, the bulb of the vestibule, and the greater vestibular glands” (Gray's Anatomy, C. D. Clemente, 13^{th} American Edition).

R. J. Levin teaches us that because “ . . . male and female genitalia develop embryologically from the common tissue anlagen, [that] male and female genital structures are argued to be homologues of one another. Thus the clitoris is the penile homologue and the labia homologues of the scrotal sac. . . . ” (Levin, R. J. (1991), Exp. Clin. Endocrinol., 98, 61-69).

Male Sexual Dysfunction (MSD)

Male sexual dysfunction (MSD) as referred to herein is meant to include ejaculatory disorders, such as anorgasmia (inability to achieve orgasm), or desire disorders, such as hypoactive sexual desire disorder (lack of interest in sex). MSD also includes male erectile dysfunction (MED).

Male Erectile Dysfunction (MED)

Male erectile dysfunction (MED), otherwise known as male erectile disorder, is defined as:
“the inability to achieve and/or maintain a penile erection for satisfactory sexual performance” (NIH Consensus Development Panel on Impotence, 1993)”

It has been estimated that the prevalence of erectile dysfunction (ED) of all degrees (minimal, moderate and complete impotence) is 52% in men 40 to 70 years old, with higher rates in those older than 70 (Melman et al 1999, J. Urology, 161, p 5-11). The condition has a significant negative impact on the quality of life of the individual and their partner, often resulting in increased anxiety and tension which leads to depression and low self-esteem. Whereas two decades ago, MED was primarily considered to be a psychological disorder (Benet et al 1994 Comp. Ther., 20: 669-673), it is now known that for the majority of individuals there is an underlying organic cause. As a result, much progress has been made in identifying the mechanism of normal penile erection and the pathophysiologies of MED.

Penile erection is a haemodynamic event which is dependent upon the balance of contraction and relaxation of the corpus cavernosal smooth muscle and vasculature of the penis (Lerner et al 1993, J. Urology, 149, 1256-1255). Corpus cavernosal smooth muscle is also referred to herein as corporal smooth muscle or in the plural sense corpus cavernosa. Relaxation of the corpus cavernosal smooth muscle leads to an increased blood flow into the trabecular spaces of the corpus cavernosa, causing them to expand against the surrounding tunica and compress the draining veins. This produces a vast elevation in blood pressure which results in an erection (Naylor, 1998, J. Urology, 81, 424-431).

The changes that occur during the erectile process are complex and require a high degree of co-ordinated control involving the peripheral and central nervous systems, and the endocrine system (Naylor, 1998, J. Urology, 81, 424-431). Corporal smooth muscle contraction is modulated by sympathetic noradrenergic innervation via activation of postsynaptic α₁-adrenoceptors. MED may be associated with an increase in the endogenous smooth muscle tone of the corpus cavernosum. However, the process of corporal smooth muscle relaxation is mediated partly by non-adrenergic, non-cholinergic (NANC) neurotransmission. There are a number of other NANC neurotransmitters found in the penis, other than NO, such as calcitonin gene related peptide (CGRP) and vasoactive intestinal peptide (VIP). The main relaxing factor responsible for mediating this relaxation is nitric oxide (NO), which is synthesised from L-arginine by nitric oxide synthase (NOS) (Taub et al 1993 Urology, 42, 698-704). It is thought that reducing corporal smooth muscle tone may aid NO to induce relaxation of the corpus cavernosum. During sexual arousal in the male, NO is released from neurones and the endothelium and binds to and activates soluble guanylate cyclase (sGC) located in the smooth muscle cells and endothelium, leading to an elevation in intracellular cyclic guanosine 3′,5′-monophosphate (cGMP) levels. This rise in cGMP leads to a relaxation of the corpus cavernosum due to a reduction in the intracellular calcium concentration ([Ca²⁺]ᵢ), via unknown mechanisms thought to involve protein kinase G activation (possibly due to activation of Ca²⁺ pumps and Ca²⁺-activated K⁺ channels).

Sildenafil citrate (also known as Viagra™) has recently been developed by Pfizer as the first oral drug treatment for MED. Sildenafil acts by inhibiting cGMP breakdown in the corpus cavernosa by selectively inhibiting phosphodiesterase 5 (PDE5), thereby limiting the hydrolysis of cGMP to 5′GMP (Boolel et al., 1996, J. Urology, 78, 257-261; Jeremy et al., 1997, Br. J. Urology, 79, 958-963) and thereby increasing the intracellular concentrations of cGMP and facilitating corpus cavernosal smooth muscle relaxation.

Currently, all other available MED therapies on the market, such as treatment with prostaglandin-based compounds i.e. alprostadil which can be administered intra-urethrally (available from Vivus Inc., as Muse™) or via small needle injection (available from Pharmacia & Upjohn, as Caverject™), are either inconvenient and/or invasive. Other treatments include vacuum constriction devices, vasoactive drug injection or penile prostheses implantation (Montague et al., 1996, J. Urology, 156, 2007-2011). Although injectable vasoactive drugs show high efficacy, side effects such as penile pain, fibrosis and priapism are common, and injection therapy is not as convenient as oral therapy. Therefore sildenafil currently represents the most preferred therapy on the market.

Thus, it is desirable to find new ways of treating male sexual dysfunction, in particular MED.

### SUMMARY ASPECTS

A seminal finding of the present invention is that dopamine D2 receptors are responsible for the adverse side effects observed following administration of non-selective dopamine receptor agonists, such as apomorphine, pramipexole and 7-hydroxy-DPAT for example. Surprisingly, it has also been shown that by selective activation of dopamine D3 receptors, treatment of sexual dysfunction, particularly of male erectile dysfunction (MED) and of female sexual dysfunction, particularly female sexual arousal disorder (FSAD) and/or hypoactive sexual desire disorder (HSDD), can be effected whilst adverse side effects, such as one or more of nausea, emesis, syncope, hypotension or bradycardia, observed following administration of non-selective agonists, are alleviated and/or substantially eliminated. We have found that in order to obtain a satisfactory treatment of sexual dysfunction whilst reducing and/or eliminating adverse side effects effectively, the dopamine D3 agonist must be selective for a dopamine D3 receptor as compared with a dopamine D2 receptor, which selectivity is, when measured using the same functional assay, at least 3-times the selectivity achieved by the control, slightly D3-preferring (non-selective) compound pramipexole. Surprisingly, the applicants have found that by using a selective dopamine D3 agonist according to the present invention, one or more of the side effects, such as nausea, vomiting and adverse cardiovascular events, observed following administration of non-selective dopamine agonists, are eliminated or substantially alleviated.

Thus, the compounds according to the present invention have the unexpected advantage of reduced adverse side effects as compared with known dopamine agonists.

### DETAILED ASPECTS

In one aspect the present invention relates to the use of a composition or a pharmaceutical composition comprising a selective dopamine D3 receptor agonist, wherein said dopamine D3 receptor agonist is functionally selective for a dopamine D3 receptor as compared with a dopamine D2 receptor, which selectivity is, when measured using the same functional assay, at least 3-times the functional selectivity achieved by the control, slightly D3-preferring (non-selective) compound pramipexole, in the manufacture or preparation of a medicament for the treatment and/or prevention of sexual dysfunction.

In other words, said selective dopamine D3 receptor agonist is functionally selective for D3 receptors over D2 receptors to an extent which, when the compound according to the present invention and the control compound pramipexole are tested using the same functional assay, i.e. with the same or substantially the same parameters, the compound according to the present invention is at least 3-times more selective compared with the functional selectivity for D3 receptors observed in respect of pramipexole.

In a further aspect the present invention relates to the use of a composition or a pharmaceutical composition comprising a selective dopamine D3 receptor agonist, but not in combination with one or more of an NEP inhibitor, a neuropeptide Y (NPY) inhibitor, a bombesin receptor antagonist or an agent capable of modulating the activity of an intermediate conductance calcium-activated potassium (IK_{Ca}) channel in the sexual genitalia of an individual, in the manufacture or preparation of a medicament for the treatment and/or prevention of sexual dysfunction.

In another aspect, the present invention relates to the use of a composition consisting essentially of a selective dopamine D3 receptor agonist in the preparation of a medicament for the treatment and/or prevention of sexual dysfunction.

In a further aspect, the present invention relates to the use of a composition consisting of a selective dopamine D3 receptor agonist in the preparation of a medicament for the treatment and/or prevention of sexual dysfunction.

In a yet further aspect, the present invention relates to a composition or a pharmaceutical composition comprising a selective dopamine D3 receptor agonist wherein said dopamine D3 receptor agonist is functionally selective for a dopamine D3 receptor as compared with a dopamine D2 receptor, which functional selectivity is, when measured using the same functional assay, at least 3-times the functional selectivity achieved by the control, slightly D3-preferring (non-selective) compound pramipexole; wherein said agonist is optionally admixed with a pharmaceutically acceptable carrier, diluent or excipient.

In a further aspect, the present invention relates to a composition or a pharmaceutical composition comprising a selective dopamine D3 receptor agonist, but not in combination with one or more of an NEP inhibitor, an NPY inhibitor, a bombesin receptor antagonist or an agent capable of modulating the activity of an intermediate conductance calcium-activated potassium (IK_{Ca}) channel in the sexual genitalia of an individual, wherein said selective dopamine D3 receptor agonist is optionally admixed with a pharmaceutically acceptable carrier, diluent or excipient.

In a further aspect, the present invention relates to a composition or a pharmaceutical composition consisting essentially of a selective dopamine D3 receptor agonist; wherein said composition is optionally admixed with a pharmaceutically acceptable carrier, diluent or excipient.

In a further aspect, the present invention relates to a composition or a pharmaceutical composition consisting of a selective dopamine D3 receptor agonist; wherein said composition is optionally admixed with a pharmaceutically acceptable carrier, diluent or excipient.

In another aspect, the present invention relates to a method of treating and/or preventing sexual dysfunction in a human or animal which method comprises administering to an individual an effective amount of a composition comprising a selective dopamine D3 receptor agonist wherein said dopamine D3 receptor agonist is functionally selective for a dopamine D3 receptor as compared with a dopamine D2 receptor, which functional selectivity is, when measured using the same functional assay, at least 3-times the functional selectivity achieved by the control, slightly D3-preferring (non-selective) compound pramipexole, wherein said selective dopamine D3 receptor agonist is optionally admixed with a pharmaceutically acceptable carrier, diluent or excipient.

In a further aspect, the present invention relates to a method of treating and/or preventing sexual dysfunction in a human or animal which method comprises administering to an individual an effective amount of a composition comprising a selective dopamine D3 receptor agonist, but not in combination with one or more of an NEP inhibitor, an NPY inhibitor, a bombesin receptor antagonist or an agent capable of modulating the activity of an intermediate conductance calcium-activated potassium (IK_{Ca}) channel in the sexual genitalia of an individual, wherein said selective dopamine D3 receptor agonist is optionally admixed with a pharmaceutically acceptable carrier, diluent or excipient.

In another aspect, the present invention provides a method of treating or preventing sexual dysfunction in a human or animal which method comprises administering to an individual an effective amount of a composition consisting essentially of a selective dopamine D3 receptor agonist, wherein said composition is optionally admixed with a pharmaceutically acceptable carrier, diluent or excipient.

In a further aspect, the present invention provides a method of treating or preventing sexual dysfunction in a human or animal which method comprises administering to an individual an effective amount of a composition consisting of a selective dopamine D3 receptor agonist, wherein said composition is optionally admixed with a pharmaceutically acceptable carrier, diluent or excipient.

There is further provided a pharmaceutical pack comprising one or more compartments wherein at least one compartment comprises one or more selective dopamine D3 receptor agonists.

In a further aspect, the present invention relates to a pharmaceutical pack comprising one or more compartments wherein at least one compartment comprises one or more compositions comprising a selective dopamine D3 receptor agonist, but not in combination with one or more of an NEP inhibitor, an NPY inhibitor, a bombesin receptor antagonist or an agent capable of modulating the activity of an intermediate conductance calcium-activated potassium (IK_{Ca}) channel in the sexual genitalia of an individual.

In a yet further aspect, the present invention provides a pharmaceutical pack comprising one or more compartments wherein at least one compartment comprises one or more compositions consisting essentially of a selective dopamine D3 receptor agonist.

In a yet further aspect, the present invention provides a pharmaceutical pack comprising one or more compartments wherein at least one compartment comprises one or more compositions consisting of a selective dopamine D3 receptor agonist.

The present invention further provides a process of preparation of a pharmaceutical composition according to the present invention, said process comprising admixing one or more selective dopamine D3 agonists with a pharmaceutically acceptable diluent, excipient or carrier.

In a further aspect, the present invention provides a process of preparation of a pharmaceutical composition according to the present invention, said process comprising admixing one or more selective dopamine D3 agonists, wherein said dopamine D3 receptor agonist is functionally selective for a dopamine D3 receptor as compared with a dopamine D2 receptor, which functional selectivity is, when measured using the same functional assay, at least 3-times the functional selectivity achieved by the control, slightly D3-preferring (non-selective) compound pramipexole with a pharmaceutically acceptable diluent, excipient or carrier.

The present invention further provides a process of preparation of a pharmaceutical composition according to the present invention, said process comprising admixing one or more compositions consisting essentially of a selective dopamine D3 receptor agonist with a pharmaceutically acceptable diluent, excipient or carrier.

The present invention further provides a process of preparation of a pharmaceutical composition according to the present invention, said process comprising admixing one or more compositions consisting of a selective dopamine D3 receptor agonist with a pharmaceutically acceptable diluent, excipient or carrier.

In a further aspect, the present invention relates to an assay method for identifying an agent (hereinafter referred to as a selective dopamine D3 agonist) that can be used to treat and/or prevent female sexual dysfunction, in particular FSAD and/or HSDD, or male sexual dysfunction, in particular MED, the assay comprising: determining whether a test agent can directly enhance the endogenous genital engorgement process or erectile process; wherein said enhancement is defined as a potentiation of genital blood flow or intracavernosal (i.c.) pressure (and/or cavernosal blood-flow) in the presence of a test agent as defined herein; such potentiation by a test agent is indicative that the test agent may be useful in the treatment and/or prevention of female sexual dysfunction, in particular FSAD and/or HSDD, or male sexual dysfunction, in particular MED and wherein said test agent is a selective dopamine D3 receptor agonist. Preferably, the agent does not cause, or causes only to a minimal degree, in an individual administered with said agent, any one of the following: nausea, emesis, syncope, hypotension or bradycardia.

In a further aspect, the present invention relates to an animal model used to identify agents capable of treating or preventing female sexual dysfunction, in particular FSAD and/or HSDD, or male sexual dysfunction, in particular MED, said model comprising an anaesthetised female or male animal including means to measure changes in vaginal/clitoral blood flow, intracavernosal pressure and/or cavernosal blood flow of said animal following stimulation of the pelvic nerve thereof; and wherein said agent in a selective dopamine D3 receptor agonist.

The animal model may further comprise means to measure the following parameters in or of said animal: nausea, emesis, syncope, hypotension or bradycardia.

In a further aspect, the present invention relates to an assay method for identifying an agent that can directly enhance the endogenous genital arousal process or erectile process in order to treat FSAD or MED, the assay method comprising: administering an agent to the animal model of the present invention; and measuring the change in the endogenous genital arousal process or erectile process; wherein said change is defined as a potentiation of vaginal/clitoral blood flow, intracavernosal pressure (ICP) (and/or cavernosal blood flow) in the animal model in the presence of a test agent as defined; and wherein said agent is a selective dopamine D3 receptor agonist.

In a further aspect, the present invention relates to a diagnostic method, the method comprising isolating a sample from a female or male; determining whether the sample contains an entity present in such an amount as to cause female sexual dysfunction, preferably FSAD and/or HSDD, or male sexual dysfunction, preferably MED; wherein the entity has a direct effect on the endogenous genital arousal process in the female or erectile process in the corpus cavernosum of the male; and wherein said entity can be modulated to achieve a beneficial effect by use of an agent; and wherein said agent is a selective dopamine D3 receptor agonist.

In a further aspect, the present invention relates to a diagnostic composition or kit comprising means for detecting an entity in an isolated female or male sample; wherein the means can be used to determine whether the sample contains the entity and in such an amount to cause female sexual dysfunction, preferably FSAD and/or HSDD, or male sexual dysfunction, preferably MED, or is in an amount so as to cause sexual dysfunction, preferably FSAD and/or HSDD, or MED; wherein the entity has a direct effect on the endogenous genital arousal process or erectile process and wherein said entity can be modulated to achieve a beneficial effect by use of an agent; and wherein said agent is a selective dopamine D3 receptor agonist.

In a further aspect, the present invention relates to a diagnostic method, the method comprising isolating a sample from a male/or female; determining whether the sample contains an entity present in such an amount as to cause sexual dysfunction; and wherein the entity can be modulated to achieve a beneficial effect by use of an agent; and wherein said agent is a selective dopamine D3 receptor agonist.

In a yet further aspect, the present invention relates to a diagnostic composition or kit comprising means for detecting an entity in an isolated male or female sample; wherein the means can be used to determine whether the sample contains the entity and in such an amount to cause sexual dysfunction; wherein said entity can be modulated to achieve a beneficial effect by use of an agent; and wherein said agent is a selective dopamine D3 receptor agonist.

For ease of reference, these and further aspects of the present invention are now discussed under appropriate section headings. However, the teachings under each section are not necessarily limited to each particular section.

The term “comprising” as used herein means that the selective dopamine D3 receptor agonist need not be the sole active component in the composition—other active and non-active components may be present.

The term “active component” as used herein means a component or ingredient which is active in the treatment of sexual dysfunction (i.e. male sexual dysfunction, preferably MED, or female sexual dysfunction, preferably FSAD and/or HSDD).

The term “consisting essentially of” as used herein means that the selective dopamine D3 receptor agonist is the sole active component in the composition. However, other non-active components may be present.

The term “consisting of” as used herein means that the selective dopamine D3 receptor agonist is the sole component in the composition, with the exception of, and limited to, any pharmaceutically acceptable carriers, diluents or excipients, as are necessary.

Each of the terms “selective dopamine D3 receptor agonist” or “selective dopamine D3 agonist” or “selective D3 agonist” or “selective D3 dopamine receptor agonist” or “selective D3 dopamine agonist” or D3 selective agonist” used herein are interchangeable and means a dopamine D3 receptor agonist which is functionally selective for a dopamine D3 receptor as compared with a dopamine D2 receptor, which functional selectivity is, when measured using the same functional assay, at least 3-times the functional selectivity achieved by the control, slightly D3-preferring (non-selective) compound pramipexole.

The term “functionally selective” as used herein in relation to the dopamine D3 receptor agonist means capable of selectively enhancing the action of, or activating a, D3 receptor as compared with a D2 receptor. Similarly, the term “functionally selective” as used herein in relation to the dopamine D3 receptor agonist means capable of selectively enhancing the action of, or activating a, D3 receptor as compared with a D1, D4 or D5 receptor.

The terms “selective” or “selectivity” as used herein in relation to compounds according to the present invention means “functionally selective” or “functional selectivity”.

The terms “preparation” and “manufacture” are to be construed synonymously in the context of the present invention (i.e. in the context of the “preparation” or “manufacture” of a medicament or the like).

Preferable Aspects

The agents for use in the treatment or prevention of sexual dysfunction according to the present invention are preferably functionally selective dopamine D3 agonists.

In one embodiment, preferably the agent for use according to the present invention is for oral administration.

In another embodiment, the agent for use according to the present invention may be for topical administration or intranasal administration.

Preferably, the agent according to the present invention is for use in the treatment or prevention of male erectile dysfuntion (MED).

Preferably, the agent according to the present invention is for use in the treatment or prevention of female sexual dysfunction (FSD).

Preferably, the agent according to the present invention is for use in the treatment or prevention of female sexual arousal disorder (FSAD).

Preferably, the agent according to the present invention is for use in the treatment or prevention of hypoactive sexual desire disorder (HSDD).

Preferably, the agent according to the present invention is for use in the treatment or prevention of female sexual arousal disorder (FSAD) and hypoactive sexual desire disorder (HSDD) (i.e. FSAD with concomitant HSDD is treated or prevented).

Preferably said selective D3 receptor agonist is functionally selective for a dopamine D3 receptor as compared with a dopamine D2 receptor, which functional selectivity is, when measured using the same functional assay, at least 5-times the functional selectivity achieved by the control, slightly D3-preferring (non-selective) compound pramipexole.

Preferably said selective D3 receptor agonist is functionally selective for a dopamine D3 receptor as compared with a dopamine D2 receptor, which functional selectivity is, when measured using the same functional assay, at least 10-times the functional selectivity achieved by the control, slightly D3-preferring (non-selective) compound pramipexole.

Preferably said selective D3 receptor agonist is functionally selective for a dopamine D3 receptor as compared with a dopamine D2 receptor, which functional selectivity is, when measured using the same functional assay, at least 20-times the functional selectivity achieved by the control, slightly D3-preferring (non-selective) compound pramipexole.

Preferably said selective D3 receptor agonist is functionally selective for a dopamine D3 receptor as compared with a dopamine D2 receptor, which functional selectivity is, when measured using the same functional assay, at least 25-times the functional selectivity achieved by the control, slightly D3-preferring (non-selective) compound pramipexole.

Preferably said selective D3 receptor agonist is functionally selective for a dopamine D3 receptor as compared with a dopamine D2 receptor, which functional selectivity is, when measured using the same functional assay, at least 30-, 40-, 50-, 60-, 70-, 80-, 90-, 100-, 110-, or at least 120-times the functional selectivity achieved by the control, slightly D3-preferring (non-selective) compound pramipexole.

Preferably said selective D3 receptor agonist exhibits a functional potency at D3 receptor expressed as an EC50, lower than 1000 nm, more preferably lower than 100 nm, yet more preferably lower than 50 nm, most preferably lower than 10 nm.

The present invention also encompasses administration of the agent of the present invention before and/or during sexual arousal/stimulation.

Thus, for some aspects of the present invention it is highly desirable that there is a sexual arousal/stimulation step.

Here, “sexual arousal/stimulation” may be one or more of a visual arousal/stimulation, a physical arousal/stimulation, an auditory arousal/stimulation or a thought arousal/stimulation.

Thus, preferably the agents of the present invention are delivered before or during sexual arousal/stimulation, particularly when those agents are for oral delivery.

Further Preferable Aspects

As noted above, it has been determined that functionally pramipexole is only about 5-fold to about 9-fold selective for D3 receptors over D2 receptors. Thus, if a selective dopamine D3 receptor agonist of the present invention is said to be “ . . . functionally selective for a dopamine D3 receptor as compared with a dopamine D2 receptor, which selectivity is, when measured using the same functional assay, at least 3-times the functional selectivity achieved by pramipexole . . . ”, then this may alternatively be expressed as the selective dopamine D3 receptor agonist of the present invention being “ . . . at least about 15-times to about 27-times more functionally selective for a dopamine D3 receptor as compared with a dopamine D2 receptor when measured using the same functional assay . . . ” (i.e. at least 3× about 5-fold (=at least about 15-times) to 3× about 9-fold (=about 27-times) functional selectivity for a dopamine D3 receptor as compared with a dopamine D2 receptor when measured using the same functional assay).

Similarly, where the functional selectivity of the D3 receptor agonist of the present invention is stated to be at least 5-times the functional selectivity achieved by pramipexole, this equates to the D3 receptor agonist being at least about 25-times to about 45-times more functionally selective for a dopamine D3 receptor as compared with a dopamine D2 receptor when measured using the same functional assay.

Similarly, where the functional selectivity of the D3 receptor agonist of the present invention is stated to be at least 10-times the functional selectivity achieved by pramipexole, this equates to the D3 receptor agonist being at least about 50-times to about 90-times more functionally selective for a dopamine D3 receptor as compared with a dopamine D2 receptor when measured using the same functional assay.

Similarly, where the functional selectivity of the D3 receptor agonist of the present invention is stated to be at least 20-times the functional selectivity achieved by pramipexole, this equates to the D3 receptor agonist being at least about 100-times to about 180-times more functionally selective for a dopamine D3 receptor as compared with a dopamine D2 receptor when measured using the same functional assay.

Similarly, where the functional selectivity of the D3 receptor agonist of the present invention is stated to be at least 25-times the functional selectivity achieved by pramipexole, this equates to the D3 receptor agonist being at least about 125-times to about 225-times more functionally selective for a dopamine D3 receptor as compared with a dopamine D2 receptor when measured using the same functional assay.

With this in mind, the present invention provides the following further (numbered) aspects:

1. The use of a composition comprising a selective dopamine D3 receptor agonist, wherein said dopamine D3 receptor agonist is at least about 15-times, preferably at least about 27-times, more preferably at least about 30-times, most preferably at least about 100-times more functionally selective for a dopamine D3 receptor as compared with a dopamine D2 receptor when measured using the same functional assay, in the preparation of a medicament for the treatment and/or prevention of sexual dysfunction. Preferably, said selective dopamine D3 receptor agonist does not cause, or causes only to a minimal degree, in an individual administered with said selective dopamine D3 receptor agonist any one of the following: nausea, emesis, syncope, hypotension or bradycardia.

2. The use of a composition according to aspect 1 consisting essentially of a selective dopamine D3 receptor agonist in the preparation of a medicament for the treatment and/or prevention of sexual dysfunction.

3. The use of a composition according to aspect 1 or aspect 2 consisting of a selective dopamine D3 receptor agonist in the preparation of a medicament for the treatment and/or prevention of sexual dysfunction.

4. The use according to any one of the preceding aspects for the treatment and/or prevention of male erectile dysfuntion (MED).

5. The use according to any one of aspects 1-3 for the treatment and/or prevention of female sexual dysfunction (FSD).

6. The use according to aspect 5 for the treatment and/or prevention of female sexual arousal disorder (FSAD) and/or hypoactive sexual desire disorder (HSDD).

7. The use according to any one of the preceding aspects wherein said medicament is administered by mouth or intranasally.

8. The use according to any one of the preceding aspects wherein said selective dopamine D3 receptor agonist is administered before and/or during sexual arousal.

9. A pharmaceutical composition comprising a selective dopamine D3 receptor agonist, wherein said dopamine D3 receptor agonist is at least about 15-times, preferably at least about 27-times, more preferably at least about 30-times, most preferably at least about 100-times more functionally selective for a dopamine D3 receptor as compared with a dopamine D2 receptor when measured using the same functional assay; and wherein said agonist is optionally admixed with a pharmaceutically acceptable carrier, diluent or excipient. Preferably, said selective dopamine D3 receptor agonist does not cause, or causes only to a minimal degree, in an individual administered with said selective dopamine D3 receptor agonist any one of the following: nausea, emesis, syncope, hypotension or bradycardia.

10. A method of treating or preventing sexual dysfunction in a human or animal which method comprises administering to an individual an effective amount of a composition comprising a selective dopamine D3 receptor agonist, wherein said dopamine D3 receptor agonist is at least about 15-times, preferably at least about 27-times, more preferably at least about 30-times, most preferably at least about 100-times more functionally selective for a dopamine D3 receptor as compared with a dopamine D2 receptor when measured using the same functional assay; and wherein said agonist is optionally admixed with a pharmaceutically acceptable carrier, diluent or excipient. Preferably, said selective dopamine D3 receptor agonist does not cause, or causes only to a minimal degree, in an individual administered with said selective dopamine D3 receptor agonist any one of the following: nausea, emesis, syncope, hypotension or bradycardia.

11. A method of treating or preventing sexual dysfunction in a human or animal according to aspect 10, which method comprises administering to an individual an effective amount of a composition consisting essentially of a selective dopamine D3 receptor agonist.

12. A method of treating or preventing sexual dysfunction in a human or animal according to aspect 10 or aspect 11, which method comprises administering to an individual an effective amount of a composition consisting of a selective dopamine D3 receptor agonist.

13. A method according to any one aspects 10-12, wherein said sexual dysfunction is male erectile dysfunction (MED).

14. A method according to any one of aspects 10-12, wherein said sexual dysfunction is female sexual dysfunction (FSD).

15. A method according to aspect 14, wherein said female sexual dysfunction (FSD) is female sexual arousal disorder (FSAD) and/or hypoactive sexual desire disorder (HSDD).

16. An assay method for identifying an agent (hereinafter referred to as a selective dopamine D3 agonist) that can be used to treat and/or prevent sexual dysfunction, the assay comprising; determining whether a test agent can directly enhance the endogenous genital engorgement process or erectile process; wherein said enhancement is defined as a potentiation of genital blood flow or intracavernosal pressure and/or cavernosal blood flow in the presence of a test agent as defined herein; such potentiation by a test agent is indicative that the test agent may be useful in the treatment and/or prevention of sexual dysfunction and wherein said test agent is a selective dopamine D3 receptor agonist. Preferably, the agent does not cause, or causes only to a minimal degree, in an individual administered with said agent any one of the following: nausea, emesis, syncope, hypotension or bradycardia.

17. An agent identified by the assay method according to aspect 16.

18. A medicament for oral or intranasal administration to treat sexual dysfunction, wherein the medicament comprises the agent according to aspect 17.

19. A diagnostic method the method comprising isolating a sample from a female or male; determining whether the sample contains an entity present in such an amount as to cause female sexual dysfunction or male sexual dysfunction; wherein the entity has a direct effect on the endogenous genital arousal process in the female or erectile process in the corpus cavernosum of the male; and wherein said entity can be modulated to achieve a beneficial effect by use of an agent; and wherein said agent is a selective dopamine D3 receptor agonist. Preferably, said selective dopamine D3 receptor agonist does not cause, or causes only to a minimal degree, in an individual administered with said selective dopamine D3 receptor agonist any one of the following: nausea, emesis, syncope, hypotension or bradycardia.

20. A diagnostic composition or kit comprising means for detecting an entity in an isolated female or male sample; wherein the means can be used to determine whether the sample contains the entity and in such an amount to cause female sexual dysfunction or male sexual dysfunction or is in an amount so as to cause sexual dysfunction; wherein the entity has a direct effect on the endogenous genital arousal process or erectile process and wherein said entity can be modulated to achieve a beneficial effect by use of an agent; and wherein said agent is a selective dopamine D3 receptor agonist. Preferably, said selective dopamine D3 receptor agonist does not cause, or causes only to a minimal degree, in an individual administered with said selective dopamine D3 receptor agonist any one of the following: nausea, emesis, syncope, hypotension or bradycardia.

21. An animal model used to identify agents capable of treating or preventing female sexual dysfunction or male sexual dysfunction, said model comprising an anaesthetised female or male animal including means to measure changes in vaginal/clitoral blood flow, intracavernosal pressure and/or cavernosal blood flow of said animal following stimulation of the pelvic nerve thereof; and wherein said agent in a selective dopamine D3 receptor agonist. Preferably, the animal model may further comprise means to measure the following parameters in or of said animal: nausea, emesis, syncope, hypotension or bradycardia.

22. An assay method for identifying an agent that can directly enhance the endogenous genital arousal process or erectile process in order to treat FSAD or MED, the assay method comprising: administering an agent to the animal model according to aspect 21; and measuring the change in the endogenous genital arousal process or erectile process; wherein said change is defined as a potentiation of vaginal/clitoral blood flow, intracavernosal pressure (ICP) (and/or cavernosal blood flow) in the animal model in the presence of a test agent as defined; and wherein said agent is a selective dopamine D3 receptor agonist. Preferably, said selective dopamine D3 receptor agonist does not cause, or causes only to a minimal degree, in an individual administered with said selective dopamine D3 receptor agonist any one of the following: nausea, emesis, syncope, hypotension or bradycardia.

23. The use of a combination consisting of one or more selective dopamine D3 receptor agonists, wherein said dopamine D3 receptor agonists are at least about 15-times, preferably at least about 27-times, more preferably at least about 30-times, most preferably at least about 100-times more functionally selective for a dopamine D3 receptor as compared with a dopamine D2 receptor when measured using the same functional assay, and one or more of the following auxiliary agents in the preparation of a medicament for the treatment or prevention of sexual dysfunction:
(i) Naturally occurring or synthetic prostaglandins or esters thereof;
(ii) α-adrenergic receptor antagonist compounds also known as α-adrenoceptors or α-receptors or α-blockers;
(iii) NO-donor (NO-agonist) compounds;
(iv) Potassium channel openers or modulators;
(v) Vasodilator agents;
(vi) Thromboxane A2 agonists;
(vii) CNS active agents;
(viii) Ergot alkoloids;
(ix) Compounds which modulate the action of naturetic factors in particular atrial naturetic factor (also known as atrial naturetic peptide), B type and C type naturetic factors such as inhibitors of neutral endopeptidase;
(x) Compounds which inhibit neutral endopeptidase (NEP);
(xi) Angiotensin receptor antagonists;
(xii) Substrates for NO-synthase;
(xiii) Calcium channel blockers;
(xiv) Antagonists of endothelin receptors and inhibitors or endothelin-converting enzyme;
(xv) Cholesterol lowering agents;
(xvi) Antiplatelet and antithrombotic agents;
(xvii) Insulin sensitising agents;
(xviii) L-DOPA or carbidopa;
(xix) Acetylcholinesterase inhibitors;
(xx) Steroidal or non-steroidal anti-inflammatory agents;
(xxi) Estrogen receptor modulators and/or estrogen agonists and/or estrogen antagonists;
(xxii) A PDE inhibitor;
(xxiii) Vasoactive intestinal protein (VIP), VIP mimetic, VIP analogue, more particularly mediated by one or more of the VIP receptor subtypes VPAC1, VPAC or PACAP (pituitory adenylate cyclase activating peptide), one or more of a VIP receptor agonist or a VIP analogue (e.g. Ro-125-1553) or a VIP fragment, one or more of a α-adrenoceptor antagonist with VIP combination (e.g. Invicorp, Aviptadil);
(xxiv) A melanocortin receptor agonist or modulator or melanocortin enhancer;
(xxv) A serotonin receptor agonist, antagonist or modulator, more particularly agonists, antagonists or modulators for 5HT1A;
(xxvi) A testosterone replacement agent or a testosterone implant;
(xxvii) Estrogen, estrogen and medroxyprogesterone or medroxyprogesterone acetate (MPA), alone or as a combination, or estrogen and methyl testosterone hormone replacement therapy agent;
(xxviii) A modulator of transporters for noradrenaline, dopamine and/or serotonin;
(xxix) A purinergic receptor agonist and/or modulator;
(xxx) A neurokinin (NK) receptor antagonist;
(xxxi) An opioid receptor agonist, antagonist or modulator, preferably agonists for the ORL-1 receptor;
(xxxii) An agonist or modulator for oxytocin/vasopressin receptors, preferably a selective oxytocin agonist or modulator;
(xxxiii) Modulators of cannabinoid receptors;
(xxxiv) A SEP inhibitor (SEPi), for instance a SEPi having an IC₅₀ at less than 100 nanomolar, more preferably, at less than 50 nanomolar;
(xxxv) A NPY inhibitor;
(xxxvi) A bombesin receptor antagonist; or
(xxxvii) An agent capable of modulating the activity of an intermediate conductance calcium-activated potassium (IK_{Ca}) channel in the sexual genitalia of an individual.

Preferably, said one or auxiliary agents are selected from:
(a) A PDE inhibitor (PDEi) e.g. PDE5i, PDE2i, PDE3i, PDE7i, PDE8i (with PDE2i and PDE5i being most preferred);
(b) Compounds which inhibit neutral endopeptidase (NEP);
(c) α-adrenergic receptor antagonist compounds also known as α-adrenoceptors or α-receptors or α-blockers, in particular α1-adrenoceptor antagonists (e.g. α1B-adrenoceptor antagonists as disclosed in U.S. 2002/0161009 A1) and α2-adrenoceptor antagonists such as yohimbine;
(d) An NPY-Y1 antagonist;
(e) Cholesterol lowering agents e.g. statins (such as atorvastatin (Lipitor™));
(f) Estrogen receptor modulators and/or estrogen agonists and/or estrogen antagonists e.g. Lasofoxifene or Raloxifene;
(g) Androgen receptor modulators and/or androgen agonists and/or androgen antagonists e.g. Tibolone;
(h) A testosterone replacement agent or a testosterone implant; and
(i) Estrogen, estrogen and medroxyprogesterone or medroxyprogesterone acetate (MPA), alone or as a combination, or estrogen and methyl testosterone hormone replacement therapy agent.

More preferably, said one or auxiliary agents are selected from:
i) One or more of testosterone, a testosterone replacement agent (inc dehydroandrostendione), testosternone (Tostrelle), dihydrotestosterone or a testosterone implant;
ii) One or more of estradiol, estrogen, estrogen and medroxyprogesterone or medroxyprogesterone acetate (M PA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone);
iii) One or more further PDE inhibitors, more particularly a PDE 2, 4, 5, 7 or 8 inhibitor, preferably PDE2 inhibitor, said inhibitors preferably having an IC50 against the respective enzyme of less than 100 nM;
iv) One or more NEP inhibitors, preferably wherein said NEP is EC 3.4.24.11 and more preferably wherein said NEP inhibitor is a selective inhibitor for EC 3.4.24.11, more preferably a selective NEP inhibitor is a selective inhibitor for EC 3.4.24.11, which has an IC₅₀ of less than 100 nM (e.g. ompatrilat, sampatrilat) suitable NEP inhibitor compounds are described in EP-A-1097719;
v) One or more of an NPY (neuropeptide Y) inhibitor, more particularly NPY1 or NPY5 inhibitor, preferably NPY1 inhibitor, preferably said NPY inhibitors (including NPY Y1 and NPY Y5) having an IC50 of less than 100 nM, more preferably less than 50 nM; and
vi) One or more estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof the preparation of which is detailed in WO 96/21656.

Most preferably, said one or auxiliary agents are selected from:
testosterone;
estradiol;
combination of testosterone and estradiol;
lasofoxifene;
combination of lasofoxifene and testosterone;
combination of lasofoxifene and estradiol;
combination of lasofoxifene, testosterone and estradiol.

In females, the generally preferred combination is a selective D3 receptor agonist according to the present invention combined with a genital vasoactive agent and/or hormone replacement therapy (HRT), estrogen(s), androgen(s), SERMs (selective estrogen receptor modulators) or SARMs (selective androgen receptor modulators). Examples of SERMs include Lasofoxifene or Raloxifene. An example of a SARM is Tibolone.

Preferably, said compounds which inhibit NEP (NEP inhibitors; NEPi) are such as described in, for example, EP 1097719 A1 or WO 02/03995 A2. Preferably, said NPY inhibitor is such as described in, for example, EP 1097718 A1 or WO 02/47670 A1. Preferably, said bombesin receptor antagonist is such as described in, for example, WO 02/40008 A2 or U.S. 2002/0058606 A1. Preferably, said agent capable of modulating the activity of an intermediate conductance calcium-activated potassium (Ik_{Ca}) channel in the sexual genitalia of an individual is such as described in, for example, WO 02/17963 A2.

Preferably, said selective dopamine D3 receptor agonist does not cause, or causes only to a minimal degree, in an individual administered with said selective dopamine D3 receptor agonist any one of the following: nausea, emesis, syncope, hypotension or bradycardia.

24. The use according to aspect 23, wherein said PDE inhibitor is a PDE 2, 3, 4, 5, 7 or 8 inhibitor.

25. The use according to aspect 24, wherein said PDE 5 inhibitor is Sildenafil.

26. The use according to any one of aspects 23-25 for the treatment of male erectile dysfunction (MED).

27. The use according to any one of aspects 23-25 for the treatment of female sexual dysfunction (FSD).

28. The use according to aspect 27 for the treatment of female sexual arousal disorder (FSAD) and/or hypoactive sexual desire disorder (HSDD).

Preferably, said selective dopamine D3 receptor agonist of the present invention is at least about 20-, 25-, 27-, 30-, 45-, 50-, 90-, 100-, 125-, 135-, 180-, 200-, 225-, 250-, 270-, 300-, 400-, 500-, or at least about 600-times more functionally selective for a dopamine D3 receptor as compared with a dopamine D2 receptor when measured using the same functional assay.

Preferably, said selective dopamine D3 receptor agonist of the present invention has little or no functional effect at non-dopamine D3 receptors such as dopamine D1, D4 or D5 receptors.

Preferably, said selective dopamine D3 receptor agonist of the present invention has no effect on dopamine D1, D4 or D5 receptors at 1×10⁻⁵ M concentration measured using a binding affinity assay.

It has been shown in studies that pramipexole has an EC₅₀ (“effective concentration 50%”—also written EC50—and defined, in this context, as “concentration of agonist required to induce 50% of maximal agonist response”) of 0.62 nM for dopamine D3 receptor and an EC₅₀ of 37.7 nM for dopamine D4 receptor measured using effective concentration (nM) for 50% reduction in cAMP levels. Thus, pramipexole is about 60-times (approximately 37.7/0.62) more functionally selective for a dopamine D3 receptor as compared with a dopamine D4 receptor when measured using the same functional assay. As noted above, the selective dopamine D3 agonist of the present invention is at least 3-times, preferably at least 5-, 10-, 20-, or 25-times more functionally selective for a dopamine D3 receptor compared with a dopamine D2 receptor when measured using the same functional assay. Accordingly, it follows that the selective dopamine D3 agonist of the present invention is at least 3×60-times, preferably at least about 5×60-, at least about 10×60-, at least about 20×60-, or at least about 25×60-times (i.e. at least about 180-times, preferably at least about 300-, at least about 600-, at least about 1200-, or at least about 1500-times) more functionally selective for a dopamine D3 receptor compared with a dopamine D4 receptor when measured using the same functional assay.

Preferably, said selective dopamine D3 receptor agonist of the present invention avoids or ameliorates dose-limiting adverse side effects. More preferably, said side effects that are avoided or ameliorated are emesis (i.e. vomiting/nausea) and/or hypotensive effects (e.g. hypotension (preferably orthostatic hypotension), reduced blood pressure, increased cardiac output, or increased heart rate (tachycardia)) and/or reduced heart rate (bradycardia). Most preferably, said selective dopamine D3 receptor agonist avoids or ameliorates said dose-limiting adverse side effects at ≧10-times, preferably ≧100-times the dose at which adverse side effects outway the beneficial (prophylactic/therapeutic) effects when compounds not encompassed by the present invention are used (e.g. non-selective dopamine receptor agonists or non-dopamine D3 receptor agonists (e.g. selective D2 receptor agonists or D3/D2 receptor agonists), such as apomorphine (non-selective dopamine agonist), pramipexole (slightly D3-preferring D3/D2 receptor agonist) or trans-[4-[(4-Phenylpiperazin-1-yl)methyl]cyclohexyl]-pyrimidin-2-ylamine (selective D2 receptor agonist) (see FIG. 1)).

Preferably, said dopamine D3 receptor agonist of the present invention increases the therapeutic window between prosexual effects (e.g. erection) and the above-noted dose-limiting side effects by ≧10-times, preferably ≧100-times that of compounds not encompassed by the present invention (e.g. non-selective dopamine receptor agonists or non-dopamine D3 receptor agonists (e.g. selective D2 receptor agonists or D3/D2 receptor agonists), such as apomorphine (non-selective dopamine agonist), pramipexole (slightly D3-preferring D3/D2 receptor agonist) or trans-[4-[(4-Phenylpiperazin-1-yl)methyl]cyclohexyl]-pyrimidin-2-ylamine (selective D2 receptor agonist) (see FIG. 1)).

The chemical structure for the selective D2 receptor agonist, trans-[4-[(4-Phenylpiperazin-1-yl)methyl]cyclohexyl]-pyrimidin-2-ylamine, is shown below:

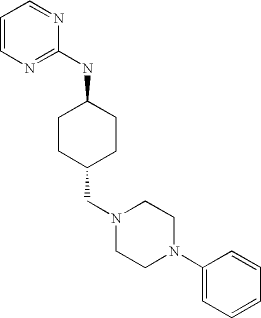

Preferably, said functional assay measures the levels of intracellular cAMP within D3 receptor-expressing cells and D2 receptor-expressing cells treated with said selective dopamine D3 receptor agonist, thereby resulting in a ratio of cAMP levels (D3 receptor-expressing cells:D2 receptor-expressing cells), with a selective dopamine D3 receptor agonist showing an at least about 15-times, preferably at least about 27-times, more preferably at least about 30-times, most preferably at least about 100-times higher level of cAMP in the D3 receptor-expressing cells compared to the D2 receptor-expressing cells. Also preferred is where said selective dopamine D3 receptor agonist shows an at least about 20-times, at least about 25-times, or an at least about 45-, 50-, 90-, 100-, 125-, 135-, 180-, 200-, 225-, 250-, 270-, 300-, 400-, 500-, or at least about 600-times higher level of cAMP in the D3 receptor-expressing cells compared to the D2 receptor-expressing cells. Preferably, said functional assay is a cAMP enzyme-linked immunoassay (enzyme immunoassay (EIA) or enzyme-linked immunosorbant assay (ELISA)), although other functional assays are readily known to the skilled person. Such a cAMP enzyme-linked immunoassay assay can be termed a “D3/D2 receptor agonist functional assay” and an example of a such an assay can be found infra. Basically, this assay measures intracellular cAMP levels from cells expressing the dopamine receptor. The human D2 family of receptor subtypes (i.e. D2, D3 and 04) acts via a Gi subtype of the G-protein to inhibit adenylate cyclase. The functional assay relies on forskolin to stimulate adenylate cyclase to increase the level of cAMP formation in the cells. If a test compound is incubated with the cells and acts as an agonist, the cAMP level will decrease as the Gi protein is being stimulated to inhibit adenylate cyclase and thus cAMP formation. The intracellular cAMP level within the cells is measured using a commercially available cAMP EIA kit (Amersham Pharmacia Biotech). The following references concerning the preferred functional assay of the present invention are incorporated herein by reference:
1. O'SULLIVAN, M. J., CAPPER, S., WHATELEY, J. HORTON, J. K., BAXENDALE, P. M., Immunoassay Applications in Life Science Research. In: Wild, D., (Ed) The Immunoassay Handbook, Nature Publishing Group, pp. 817-845, 2001.
2. HORTON, J. K., SEDDON, L. J., WILLIAMS A. S. BAXENDALE P. M. A method for measuring intracellular cyclic AMP levels with immunoassay technology and novel, cellular lysis reagents. Br. J. Pharmacol. (Suppl), 123, p. 31, 1998.
3. Conversion of the cAMP direct screening assay to a 384 well format. Amersham Biosciences, Proximity Headlines 4, 1998.
4. ICHIKAWA ET AL., Identification and role of adenylyl cyclase in auxin signalling in higher plants. Nature, 390, pp. 698-701, 1997.
5. Direct quantitation of intracellular levels of cAMP-eliminating lengthy extraction processes. Nycomed Amersham, Life Science News 23, 1997.
6. Direct and in situ measurement of cAMP in cell culture with scintillation proximity radioimmunoassay. Amersham International, Proximity News 34, 1997.
7. High throughput screening for c AMP formation by scintillation proximity radioimmunoassay. Amersham International, Proximity News 23, 1996.
8. HORTON, J. K., BAXENDALE, P. M., Mass measurements of cyclic AMP formation by radioimmunoassay, enzyme immunoassay and scintillation proximity assay. In: Kendall, D. A., and Hill, S. J., (Eds) Meth. in Mol. Biol, 41, pp. 95-105, 1995.
9. HORTON, J. K., SMITH, L., ALI, A., BAXENDALE, P. M., NEUMANN, K., KOLB, A., High throughput screening for cAMP formation by scintillation proximity radioimmunoassay. Packard Instrument Company TopCount Topics 21, pp. 1-4, 1995.

10. HORTON, J. K., MARTIN, R. C., KALINKA, S., CUSHING, A. KITCHER, J. P., O'SULLIVAN, M. J., BAXENDALE, P. M. Enzymeimmunoassays for the estimation of adenosine 3′,5′cyclic monophosphate and guanosine 3′,5′cyclic monophosphate in biological fluids. J. Immunological Methods, 155, pp. 31-40, 1992.

Another suitable functional assay can be found in Perachon et al. (1999), European Journal of Pharmacology, 366, 293-300.

Another suitable functional assay can be found in co-pending patent application EP 02257707.6 filed by the applicant on 6 Nov. 2002 and incorporated herein by reference. The assay disclosed in EP 02257707.6 is an assay method for determining activation by an agonist compound of a G-protein linked receptor, such as a neuroreceptor, said method being based on use of a Fluorometric Imaging Plate Reader (FLIPR), which comprises:
(a) generating a cell line having at least one suitable selection factor, selected from a drug resistance marker, selected from HEK293-G alpha 15, said cell line stably expressing a promiscuous G protein selected from G alpha 15, and then co-expressing said G-linked receptor in said cell line, by transfecting cDNA coding for the selected G-linked receptor, into said cell line;
(b) growing the co-expressed cells in a suitable medium;
(c) plating said cells for approximately one day;
(d) loading the plated cells with an amount of a fluorescent dye suited to the purpose;
(e) incubating the dye-loaded cells at a temperature from about room temperature to about 37° C. for about one hour;
(f) washing the plate to remove excess dye with a suitable buffer and replacing the volume of buffer removed with a similar volume of fresh buffer;
(g) incubating at from about 30° C. to about 37° C.;
(h) adding an agonist compound under constant temperature conditions from about 30° C. to about 37° C.; and
(i) measuring fluorescence emission under constant temperature conditions from about 30° C. to about 37° C. in a Fluorometric Imaging Plate Reader so as to thereby determine the level of activation of the selected receptor by the agonist compound.

In one embodiment of the assay, the G-linked receptor is a dopamine or histamine receptor. In other embodiments, the G-linked receptor is selected from the group consisting of D2, D3, Alpha 1A, Alpha 2A, M1, H1, 5HT1A, and 5HT2A receptors. In another particular embodiment, said G-linked receptor is a dopamine D3 receptor. In a further embodiment of the assay, the selection factor selected from a drug resistance marker is a puromycin-resistance marker. In yet another embodiment of the assay, the selection factor selected from a drug resistance marker is a blastocidin-resistance marker. A preferred fluorescent dye used in practising the assay method of EP 02257707.6 is Fluo-3™ or Fluo-4™. Preferably, the plated cells have a density of between about 12,000 and about 30,000 cells/square cm. In another embodiment of the assay, incubating step (g) occurs for from about 15 minutes to about 60 minutes. Preferably, said incubating step (g) occurs for about 30 minutes.

Surprising and Unexpected Findings

The present invention demonstrates the surprising and unexpected findings that:
(a) activating or stimulating dopamine D2 receptors is responsible for causing adverse side effects such as nausea, emesis, syncope, hypotension or bradycardia;
(b) selective activation or stimulation of dopamine D3 receptors using a selective dopamine D3 receptor agonist effectively treats or prevents sexual dysfunction, both male and female, in particular MED and FSAD and/or HSDD, without causing adverse side effects observed following administration of non-selective dopamine agonists. In essence, agonism of the D3 receptor is an initiator of sexual behaviour.
   Advantages

The present invention is advantageous because:
(a) selectively targeting dopamine D3 receptors by use of a selective dopamine D3 receptor agonist results in the treatment or prevention of sexual dysfunction (both male and female), particularly in the treatment or prevention of MED and FSAD and/or HSDD, whilst effectively reducing or eliminating one or more adverse side effects, such as nausea, emesis, syncope, hypotension or bradycardia observed following the administration of non-selective dopamine agonists.
   Patient Groups
   Female

The compounds of the invention find application in the following sub-populations of patients with FSD: the young, the elderly, pre-menopausal, peri-menopausal, or post-menopausal women with or without hormone replacement therapy.

The compounds of the invention find application in patients with FSD arising from:—
i) Vasculogenic etiologies e.g. cardiovascular or atherosclerotic diseases, hypercholesterolemia, cigarette smoking, diabetes, hypertension, radiation and perineal trauma, or traumatic injury to the iliohypogastric pudendal vascular system;
ii) Neurogenic etiologies such as spinal cord injuries or diseases of the central nervous system including multiple sclerosis, diabetes, Parkinsonism, cerebrovascular accidents, peripheral neuropathies, trauma or radical pelvic surgery;
iii) Hormonal/endocrine etiologies such as dysfunction of the hypothalamic/pituitary/gonadal axis, or dysfunction of the ovaries, dysfunction of the pancreas, surgical or medical castration, androgen deficiency, high circulating levels of prolactin e.g. hyperprolactinemia, natural menopause, premature ovarian failure, or hyper- and hypothyroidism;
iv) Psychogenic etiologies such as depression, obsessive compulsive disorder, anxiety disorder, postnatal depression/“Baby Blues”, emotional and relational issues, performance anxiety, marital discord, dysfunctional attitudes, sexual phobias, religious inhibition or traumatic past experiences; or
v) Drug-induced sexual dysfunction resulting from therapy with selective serotonin reuptake inhibitors (SSR is) and other antidepressant therapies (tricyclics and major tranquillizers), anti-hypertensive therapies, sympatholytic drugs, or chronic oral contraceptive pill therapy.
   Male

Patients with mild to moderate MED should benefit from treatment with the compounds according to the present invention, and patients with severe MED may also respond. However, early investigations suggest that the responder rate of patients with mild, moderate and severe MED may be greater with a selective D3 agonist/PDE5 inhibitor (PDE5i) combination. Mild, moderate and severe MED will be terms known to the man skilled in the art, but guidance can be found in The Journal of Urology, vol. 151, 54-61 (January 1994).

Early investigations suggest the below-mentioned MED patient groups should benefit from treatment with a selective D3 agonist and a PDE5i (or other combination set out hereinafter). These patient groups, which are described in more detail in Clinical Andrology vol. 23, no. 4, p773-782 and chapter 3 of the book by 1. Eardley and K. Sethia “Erectile Dysfunction-Current Investigation and Management”, published by Mosby-Wolfe, are as follows: psychogenic, organic, vascular, endocrinologic, neurogenic, arteriogenic, drug-induced sexual dysfunction (lactogenic) and sexual dysfunction related to cavernosal factors, particularly venogenic causes.

Dopamine D3 Receptor

As indicated above, the agent may be any suitable agent that can act as a selective dopamine D3 receptor agonist.

Background teachings on dopamine D3 receptors have been prepared by Victor A. McKusick et al. on http://www3.ncbi.nlm.nih.gov/Omim/searchomim.htm. The following text concerning D3 receptors has been extracted from that source:

“Sokoloff et al. (1990) [Nature 347:146-151] characterized a dopamine receptor that differs in its pharmacology and signaling system from the D1 and D2 receptors and represents both an autoreceptor and a postsynaptic receptor. Designated the dopamine receptor D3, it is localized to limbic areas of the brain, which are associated with cognitive, emotional, and endocrine functions. It appeared to mediate some of the effects of antipsychotic drugs and drugs used in the treatment of Parkinson disease, which were previously thought to interact only with D2 receptors. By screening cDNA and genomic libraries using a combination of reverse transcription and PCR, Sokoloff et al. (1990) ibid cloned the DRD3 gene. Like the DRD2 gene, but unlike most other members of this superfamily, the DRD3 gene contains introns, 5 in number. The position of 2 of the introns corresponds to that of introns in DRD2. Le Coniat et al. (1991) [Hum. Genet. Sep; 87(5):618-20] assigned the DRD3 gene to chromosome 3 by hybridization of a genomic probe to flow-sorted chromosomes and localized it to band 3q13.3 by in situ hybridization.”

The D3 receptor was initially cloned from a rat cDNA library by Sokoloff et al. (1990) ibid using probes derived from the D2 dopamine receptor sequence. The cloning of the human D3 receptor was reported shortly thereafter (Giros et al. (1990) CR Acad. Sci. III, 311: 501-508), followed by the murine D3 receptor (Fishburn et al. (1993) J. Biol. Chem. 268: 5872-5878).

Dopamine D3 Receptor Sequence Data

Nucleotide sequences and amino acid sequences for the dopamine D3 receptors are available in the literature (see Sokoloff et al. (1990); Giros et al. (1990); and Fishburn et al. (1993) ibid).

A nucleotide sequence (SEQ ID NO: 1) and an amino acid sequence (SEQ ID NO: 2) for the human dopamine D3 receptor are presented in the List of Sequences infra.

Selective D3 Agonists

A selective dopamine D3 receptor agonist is a compound which initiates a physiological response when combined with a dopamine D3 receptor and which is selective for a dopamine D3 receptor as compared with a dopamine D2 receptor, which selectivity is, when measured using the same functional assay, at least 3-times the selectivity achieved by the compound pramipexole. That is to say, a selective dopamine D3 receptor agonist according to the present invention is one which elicits a response which is at least 3-times more selective towards a D3 receptor than the compound pramipexole.

Details of suitable assay systems for identifying and/or studying dopamine D3 receptor agonists are presented hereinafter in the section entitled “D3 Agonist Assay”.

Examples of suitable selective dopamine D3 receptor agonists and intermediates relating thereto are presented in Examples section (“Chemistry Examples”) infra.

D3 Agonist Assays

As stated above, with dopamine D3 receptors and dopamine D2 receptors, binding data or binding selectivity data has been shown to not always correlate with or reflect functional data or functional selectivity data. In the present invention, by “selective” we mean “functionally selective”.

D3/D2 Agonist Binding Assay

Gonazalez et al. (Eur. J. Pharmacology 272 (1995) R1-R3) discloses an assay for determining the binding capability of a compound at D3 and/or D2 dopamine receptors and thus the binding selectivity of such compounds. This assay is, thus, herein referred to as a binding assay.

D3/D2 Agonist Functional Assay

A suitable assay for determining functionally the activity of a compound at D3 and/or D2 dopamine receptors is detailed hereinbelow.

Compounds are evaluated as agonists or antagonists at the dopamine D2 and D3 receptors by looking at cAMP levels in a GH4C1 and CHO cell-line expressing the human D2 and D3 receptors, respectively.

### EXPERIMENTAL PROCEDURE

Materials

Cell Culture Media:

| HD2LhD₂LGH4C1 Medium | hD₃CHO Medium |
|---|---|
| Hams F-10 (Sigma N6013) | DMEM, high glucose (Sigma D5671) |
| 2 mM L-Glutamine (Sigma G7513) | 2 mM L-Glutamine (Sigma G7513) |
| 10% FBS (Gibco 10106-169) | 10% dialyzed FBS (Sigma F0392) |
| 700 μg/ml Geneticin | 20 nM Methotrexate hydrate |
| (Gibco 10131-019) | (Sigma M8407) |

Two adherent cell lines expressing cloned human dopamine receptors are:
hD₂LGH4C1—rat pituitary cells expressing the human dopamine D2 long receptor; and
hD₃CHO—Chinese hamster ovary cells deficient in dihydrofolate reductase gene which express the human dopamine D3 receptor.

Media required for their growth is made up fresh every week as below and filtered through a 0.22 μM filter before use. Media stored at 4° C. and warmed to 37° C. for addition to the cells.

Cell Dissociation Solution:

(Sigma C-5914) 10-15 ml used to harvest cells from 225 cm² flask (37° C. 5 min for hD2LGH4C1 cells and 10 minutes for hD3CHO cells).

KRH Buffer:

KRH is prepared as follows:

| KH₂PO₄ | (BDH - 1025034B) | 1.2 | mM | 163 | mg/l |
|---|---|---|---|---|---|
| NaCl | (Fisher - S/3160/60) | 1.45M | | 8.47 | g/l |
| KCl | (Sigma - P-9333) | 5 | mM | 373 | mg/l |
| MgSO₄ | (BDH - 101514Y) | 1.2 | mM | 296 | mg/l |
| CaCl₂ | (Sigma - C-5080) | 1 | mM | 147 | mg/l |
| Hepes | (Sigma - H-7523) | 25 | mM | 5.96 | g/l |
| Glucose | (BDH - 101176K) | 5 | mM | 0.9 | g/l |

Made up to 1 litre with distilled water and pH adjusted to pH 7.4 at room temperature. Stored for up to 1 week at 4° C.

3-isobutyl-1-methylxanthine (IBMX):

(Sigma 17018) Dissolved to a concentration of 100 mM in DMSO. 10× assay stock of 1 mM made by carrying out a 1:100 dilution in KRH buffer. 20 μl added to a final assay volume of 2001 μl, giving a final assay concentration of 100 μM/well.

Forskolin:

(Calbiochem 344273) Dissolved to a concentration of 10 mM in water. (This stock is stored at +4° C.). 10× assay stock of 100 μM and 200 μM made by carrying out a 100- and 50-fold dilution in KRH buffer. 20111 added to a final assay volume of 200 μl, giving a final assay concentration of 10 μM for the D2 cells and 20 μM for the D3 cells.

Test Compounds:

Dissolved to a concentration of 10 mM in 100% DMSO and diluted in KRH buffer to give the top concentration of 100 μM/well in 1% DMSO/KRH (10 μM/well in 0.1% DMSO/KRH in assay). Further dilutions are made in 1% DMSO/KRH (10× assay concentration): 10 μM, 1 μM, 100 nM, 10 nM, 1 nM, 0.1 nM, 0.01 nM and 0.001 nM.

20 μl added to a final assay volume of 200 μl, giving the following final assay concentrations: 1 μM, 100 nM, 10 nM, 1 nM, 0.1 nM, 0.01 nM and 0.001 nM.

Compounds are normally assayed from 1 e-5 to 1 e-12.

The following compounds are always included in the assay:
Apomorphine
Assayed from 1 e-5 to 1 e-12
Full agonist

cAMP Enzymeimmunoassay:

All materials are supplied by Amersham Pharmacia Biotech cAMP EIA kit (RPN 225) unless otherwise stated.

Microtitre Plate:

96 well plate coated with donkey anti-rabbit IgG.

Assay Buffer:

0.05M sodium acetate buffer, pH 5.8 containing 0.02% bovine serum albumin and 0.01% preservative upon dilution. The contents of this bottle are transferred to a graduated cylinder using 3×15 ml distilled water washes. The final volume is then adjusted to 500 ml.

cAMP Standard (for Non-Acetylation Assay):

cAMP at 3200 fmol/ml upon reconstitution. Standard is dissolved in 2 ml lysis reagent 1B (see below) for use.

Antibody:

Rabbit anti-cAMP. Antibody is dissolved in 11 ml lysis reagent 2B (see below) for use.

Peroxidase Conjugate:

cAMP-horseradish peroxidase. Peroxidase conjugate is dissolved in 11 ml assay buffer for use.

Wash Buffer:

0.01M phosphate buffer, pH 7.5 containing 0.05% Tween 20 on dilution. The contents of this bottle are transferred to a graduated cylinder using 3×15 ml distilled water washes. The final volume is then adjusted to 500 ml.

TMB Substrate:

3,3′,5,5′-tetramethylbenzidine (TMB)/hydrogen peroxide, in 20% (v/v) dimethylformamide.

Lysis Reagent 1:

Dodecyltrimethlyammonium bromide (25 mg/ml on reconstituion). The powder is transferred to a 100 ml graduated cylinder using 3×15 ml assay buffer. The volume is adjusted to 60 ml and stirred until dissolved. The final volume is then made up to 80 ml with assay buffer.

Lysis Reagent 1B:

5 ml of lysis reagent 1 is diluted to 50 ml with assay buffer.

Lysis Reagent 2:

Solid, 5 g. Contains no chemicals classified as hazardous. The powder is transferred to a 100 ml graduated cylinder using 3×15 ml assay buffer. The volume is adjusted to 80 ml and stirred until dissolved. The final volume is then made up to 100 ml with assay buffer.

Lysis Reagent 2B:

10 ml of lysis reagent 2 is diluted to 40 ml with assay buffer.

Sulphuric Acid (1M):

1M Sulphuric acid is prepared from an 18M stock (BDH). 11 ml of acid is added to 189 ml of distilled water.

Specific Equipment

Spectroscophometric plate reader (Spectra max 190).

Microtitre plate shaker/incubator (Wesbart).

Methods

Resuscitation of Frozen Ampoules:

Remove ampoules from liquid nitrogen and allow them to equilibrate for 2 minutes as trapped gas or liquid may cause the ampoule to expand rapidly and explode. They can also be placed at minus 20° C. before thawing.

Thaw ampoules quickly and completely at 37° C. in a water bath.

Transfer the contents to a 15 ml tube carefully. Slowly add 2 ml of media and then another 8 ml.

Transfer cell suspension to a T25 flask and incubate for 24 h at 37° C., 5% CO₂. N.B. hD₃CHO cells can be placed straight into a T225 flask as they are fast growing cells; the amount of medium required is 50 ml.

Cell Harvesting and Splitting:

Generally, cells are split on a Monday and Wednesday in order to perform assays on Tuesday and Thursday. Cells may also be split on Friday if too confluent to leave over the weekend. It is very important not to let the hD₃CHO cells grow beyond 80% confluency as they cannot be recovered once grown past this point.

Cells are grown in T225 flasks (Jumbos). Every component added to the cells must be warmed to 37° C. before use.

Cell Harvest:

Growth media removed from flasks and cells washed twice with warm PBS (Gibco. 14040-091) and removed.

1. Approximately 10 ml of cell dissociation buffer (Sigma C5914) added to cells and placed in incubator for approx. 5 min. (D₃ cells adhere more strongly to the flask than D₂ cells, therefore D₃ cells may require longer to dislodge).

2. Flasks given a sharp tap when removed from the incubator to dislodge any remaining cells from the bottom.

3. Approximately 10 ml of full medium added to the cells and used to wash the sides of the flask. Cells are centrifuged for 5 min at 1000 rpm to pellet the cells.

4. Media is discarded and 10 ml of fresh medium used to resuspend the cells. 100 μl removed and combined with 100 μl of trypan blue (Sigma T8154) for counting.

Split Ratios:

hD₂LGH4C1 split between 1:3 to 1:6.

hD₃CHO split between 1:5 to 1:10 (faster growing of the two cell lines).

Seeding for Assay:

Require 50,000 cells/200 μl/well equal to 2.5×10⁵ cells/ml. Dilute cells to 2.5×10⁵ cells/ml and add 200 μl to wells in a tissue culture 96 well plate. Leave all cells at 37° C., 5% CO₂.

Cryopreservation of Cell Lines:

It is a good idea to create a cell bank of your own cells to resuscitate for further use.

1. Cells are harvested in the same manner as before.

2. Cells are counted.

3. Freeze medium contains full medium plus 10% DMSO, cells resuspended to give between 2 to 4×10⁶ cells/ml. Cell suspension is divided into 1 ml aliquots.

4. The cells are frozen down between 1° C. to 3° C. using ‘Mr Frosty’ in the minus 80° C. freezer overnight before being transferred to a gaseous phase nitrogen storage vessel.

It is advisable to test the cell viability by thawing one ampoule after freezing. Viabilities below 70% may cause problems on recovery due to low cell numbers and the presence of debris.

Measurement of Intracellular cAMP Levels in Cells:

Cells are plated at 50,000 cells/well into sterile 96-well plates in cell culture medium (see above) at a final volume of 200 μl/well the previous day and incubated at 37° C., 5% CO₂ overnight (O/N).

KRH buffer is made up as shown above and warmed to 37° C.

IBMX, Forskolin and test compounds are made up and diluted as shown above.

Cells are washed once with 200 μl KRH buffer.

The following are added to each well:

| HD2LhD₂GH4C1 cells | | hD₃CHO cells | |
|---|---|---|---|
| 120 | μl KRH buffer | 120 | μl KRH buffer |
| 20 | μl IMBX (100 μM/well) | 20 | μl IMBX (100 μM/well) |
| 20 | μl Forskolin (10 μM/well) | 20 | μl Forskolin (20 μM/well) |
| 20 | μl Agonist | 20 | μl Agonist |
| 20 | μl Antagonist or 1% DMSO | 20 | μl Antagonist or 1% DMSO |

Controls:

| Forskolin only | Blank | DMSO control | Antagonist control |
|---|---|---|---|
| 160 μl KRH buffer | 200 μl | 120 μl KRH buffer | 120 μl KRH buffer |
| 20 μl IMBX | KRH | 20 μl IMBX | 20 μl IMBX |
| (100 μM/well) | | (100 μM/well) | (100 μM/well) |
| 20 μl Forskolin | | 20 μl Forskolin | 20 μl Forskolin |
| (10 μM/well) | | (10 μM/well) | (10 μM/well) |
| | | 40 μl 1% DMSO | 20 μl 1% DMSO |
| | | | 20 μl Antagonist |

The plates are shaken at 37° C. for 45 mins.

After 45 min the assay mixture is aspirated and 200 μl of lysis reagent 1B is added to the cells.

Cells are shaken for 20 min before further lysing by repeated pipetting (˜20 times/well).

cAMP Enzymeimmunoassay:

Stock reagents equilibrated to room temperature and working solutions prepared (as described above).

cAMP standards prepared in eppendorf tubes labelled 12.5, 25, 50, 100, 200, 400, 800, 1600 and 3200 fmol. 0.5 ml of lysis reagent 1B is added to each tube. 0.5 ml of the diluted standard is added to the 3200 fmol tube. The tube is vortexed and 0.5 ml added to the 1600 fmol tube. This is continued to give the other dilutions.

20 μl (hD₂) and 100 μl (hD₃) of each cell lysate is transferred to the EIA plate and, for the hD2 sample, made up to 100 μl with lysis reagent 1B. No further addition is added to the hD3 sample. 100 μl of each standard and of the original standard is placed in duplicate into the plate. The following controls are set up:
Zero standard: 100 μl lysis reagent 1B
NSB: 100 μl lysis reagent 1B and 2B
Blank: no additions

100 μl of antibody is added to all wells except for blank and NSB wells before incubating for 2 hours at 4° C.

After incubation, 50 μl of peroxidase conjugate is added to all wells except for the blank wells and incubated for a further hour at 4° C.

Plates are emptied by blotting onto absorbent paper and washed 4 times with 400 μl of wash buffer. 150 μl of TMB substrate is then added to each well.

Plates are shaken at room temperature for 30 min before the addition of 100 μl of 1M sulphuric acid into all wells.

The optical density is read on Spectramax 190 at 450 nM within 30 minutes.

Calculations

Calculations are carried out using a combination of Excel and Origin templates.

The standard curve is generated by plotting percentage of control OD data (y axis) against log cAMP α-axis) mol/well in Excel. Standard curve is constrained through 0 and 100.

From standard curve, cAMP predictions are made for each sample well using the variables generated from the standard curve.

Formula for predicting a dose given a response from a sigmoid curve.

$$x = {c ( \frac{y - a}{d - y} )}^{1 / b}$$

where:—
a=lower asymptote
b=hill slope
c=IC₅₀
d=upper asymptote

cAMP predictions are made for each OD reading and expressed as a percentage of DMSO control.

Plotting Log concentration of compound α-axis) against percentage control response (y-axis), a sigmoidal dose response curve can be constructed from which an EC₅₀ concentration can be obtained.

Combinations

In more detail, the present invention further comprises the combination of a compound of the invention for the treatment of sexual dysfunction as outlined herein (more particularly male sexual dysfunction, in particular MED, or female sexual dysfunction, in particular FSAD and/or HSDD) with one or more auxiliary active agents (see later discussion for suitable examples). The combination provides a treatment for both male and female sexual dysfunction and in particular erectile dysfunctions of organic, vascular, neurogenic, drug induced and/or psychogenic origin.

The present invention further comprises the use of a combination consisting essentially of a selective dopamine D3 receptor agonist according to the present invention and two auxiliary active agents (see later discussion for suitable examples) in the manufacture or preparation of a medicament for the treatment or prevention of male sexual dysfunction as outlined herein (more particularly male erectile dysfunction (MED) or female sexual arousal disorder (FSAD) and/or hypoactive sexual desire disorder (HSDD)). The combination provides a treatment for sexual dysfunctions of organic, vascular, neurogenic, drug induced and/or psychogenic origin.

The present invention further comprises the use of a combination consisting of a selective dopamine D3 receptor agonist according to the present invention and two auxiliary active agents (see later discussion for suitable examples) in the manufacture or preparation of a medicament for the treatment or prevention of sexual dysfunction as outlined herein (more particularly male erectile dysfunction (MED) or female sexual arousal disorder (FSAD) and/or hypoactive sexual desire disorder (HSDD)). The combination provides a treatment for erectile dysfunctions of organic, vascular, neurogenic, drug induced and/or psychogenic origin.

The present invention further comprises the use of a combination consisting essentially of a selective dopamine D3 receptor agonist according to the present invention and one auxiliary active agent (see later discussion for suitable examples) in the manufacture or preparation of a medicament for the treatment or prevention of male sexual dysfunction as outlined herein (more particularly male erectile dysfunction (MED) or female sexual arousal disorder (FSAD) and/or hypoactive sexual desire disorder (HSDD)). The combination provides a treatment for sexual dysfunctions of organic, vascular, neurogenic, drug induced and/or psychogenic origin.

The present invention further comprises the use of a combination consisting of a selective dopamine D3 receptor agonist according to the present invention and one auxiliary active agent (see later discussion for suitable examples) in the manufacture or preparation of a medicament for the treatment or prevention of male sexual dysfunction as outlined herein (more particularly male erectile dysfunction (MED) or female sexual arousal disorder (FSAD) and/or hypoactive sexual desire disorder (HSDD)). The combination provides a treatment for sexual dysfunctions of organic, vascular, neurogenic, drug induced and/or psychogenic origin.

In males, and in some instances in females, preferably the combination of a selective dopamine D3 receptor agonist and one or more auxiliary agent does not comprise one or more of the following: an NEP inhibitor, and NPY inhibitor, a bombesin receptor antagonist or an agent capable of modulating the activity of an intermediate conductance calcium-activated potassium (IK_{Ca}) channel in the sexual genitalia of the individual.

In females only, it is envisaged that in some instances the selective dopamine D3 receptor agonist may be used in combination with one or both of an NEP inhibitor or NPY inhibitor. Thus, the present invention encompasses, for females only, use of such combinations to treat female sexual dysfunction (for example FSAD and/or HSDD). The present invention further provides the use of pharmaceutical compositions comprising, or consisting of, a selective dopamine D3 receptor agonist together with one, alternatively with both, of an NEP inhibitor and/or NPY inhibitor, for use in the treatment of female sexual dysfunction (for example FSAD and/or HSDD). Suitably, such a pharmaceutical composition may further comprise one or more auxiliary agents (see later discussion for suitable examples).

Thus, a further combination aspect of the invention provides a pharmaceutical combination (for simultaneous, separate or sequential administration) comprising a compound of the invention and one or more auxiliary active agents (see later discussion for suitable examples).

A yet further combination aspect of the invention provides a pharmaceutical composition (for simultaneous, separate or sequential administration) consisting essentially of a selective dopamine D3 agonist and two auxiliary active agents (see later discussion for suitable examples).

A yet further combination aspect of the invention provides a pharmaceutical composition (for simultaneous, separate or sequential administration) consisting of a selective dopamine D3 agonist and two auxiliary active agents (see later discussion for suitable examples).

A yet further combination aspect of the invention provides a pharmaceutical composition (for simultaneous, separate or sequential administration) consisting essentially of a selective dopamine D3 agonist and one auxiliary active agent (see later discussion for suitable examples).

A yet further combination aspect of the invention provides a pharmaceutical composition (for simultaneous, separate or sequential administration) consisting of a selective dopamine D3 agonist and one auxiliary active agent (see later discussion for suitable examples).

Auxiliary Active Agents

Suitable auxiliary active agents for use in the combinations of the present invention include:
1) Naturally occurring or synthetic prostaglandins or esters thereof. Suitable prostaglandins for use herein include compounds such as alprostadil, prostaglandin E₁, prostaglandin E₀, 13, 14-dihydroprosta glandin E₁, prostaglandin E₂, eprostinol, natural synthetic and semi-synthetic prostaglandins and derivatives thereof including those described in WO-00033825 and/or U.S. Pat. No. 6,037,346 issued on 14 Mar. 2000 all incorporated herein by reference, PGE₀, PGE₁, PGA₁, PGB₁, PGF₁ α, 19-hydroxy PGA₁, 19-hydroxy-PGB₁, PGE₂, PGB₂, 19-hydroxy-PGA₂, 19-hydroxy-PGB₂, PGE₃α, carboprost tromethamine dinoprost, tromethamine, dinoprostone, lipo prost, gemeprost, metenoprost, sulprostune, tiaprost and moxisylate;
2) α-adrenergic receptor antagonist compounds also known as α-adrenoceptors or α-receptors or α-blockers. Suitable compounds for use herein include: the α-adrenergic receptor blockers as described in PCT application WO99/30697 published on 14 Jun. 1998, the disclosures of which relating to α-adrenergic receptors are incorporated herein by reference and include, selective α₁-adrenoceptor or α₂-adrenoceptor blockers and non-selective adrenoceptor blockers, suitable α₁-adrenoceptor blockers include: phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, Recordati 15/2739, SNAP 1069, SNAP 5089, RS17053, SL 89.0591, doxazosin, terazosin, abanoquil and prazosin; α₂-blocker blockers from U.S. Pat. No. 6,037,346 [14 Mar. 2000] dibenamine, tolazoline, trimazosin and dibenamine; α-adrenergic receptors as described in US patents: U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptor blockers include: clonidine, papaverine, papaverine hydrochloride, optionally in the presence of a cariotonic agent such as pirxamine;
3) NO-donor (NO-agonist) compounds. Suitable NO-donor compounds for use herein include organic nitrates, such as mono- di or tri-nitrates or organic nitrate esters including glyceryl trinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl penicilliamine (SNAP) S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates, (NONOates), 1,5-pentanedinitrate, L-arginene, ginseng, zizphi fructus, molsidomine, Re-2047, nitrosylated maxisylyte derivatives such as NMI-678-11 and NMI-937 as described in published PCT application WO 0012075;
4) Potassium channel openers or modulators. Suitable potassium channel openers/modulators for use herein include nicorandil, cromokalim, levcromakalim, lemakalim, pinacidil, cliazoxide, minoxidil, charybdotoxin, glyburide, 4-amini pyridine, BaCl₂;
5) Vasodilator agents. Suitable vasodilator agents for use herein include nimodepine, pinacidil, cyclandelate, isoxsuprine, chloroprumazine, halo peridol, Rec 15/2739, trazodone;
6) Thromboxane A2 agonists;
7) CNS active agents;
8) Ergot alkoloids; Suitable ergot alkaloids are described in U.S. Pat. No. 6,037,346 issued on 14 Mar. 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride;
9) Compounds which modulate the action of natruretic factors in particular atrial naturetic factor (also known as atrial naturetic peptide), B type and C type naturetic factors such as inhibitors of neutral endopeptidase;
10) Compounds which inhibit neutral endopeptidase (NEP);
11) Angiotensin receptor antagonists such as losartan;
12) Substrates for NO-synthase, such as L-arginine;
13) Calcium channel blockers such as amlodipine;
14) Antagonists of endothelin receptors and inhibitors or endothelin-converting enzyme;
15) Cholesterol lowering agents such as statins (e.g. atorvastatin/Lipitor™) and fibrates;
16) Antiplatelet and antithrombotic agents, e.g. tPA, uPA, warfarin, hirudin and other thrombin inhibitors, heparin, thromboplastin activating factor inhibitors;
17) Insulin sensitising agents such as rezulin and hypoglycaemic agents such as glipizide;
18) L-DOPA or carbidopa;
19) Acetylcholinesterase inhibitors such as donezipil;
20) Steroidal or non-steroidal anti-inflammatory agents;
21) Estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof the preparation of which is detailed in WO 96/21656;
23) A PDE inhibitor, more particularly a PDE 2, 3, 4, 5, 7 or 8 inhibitor, preferably PDE2 or PDE5 inhibitor and most preferably a PDE5 inhibitor (see hereinafter), said inhibitors preferably having an IC50 against the respective enzyme of less than 100 nM (with the proviso that PDE 3 and 4 inhibitors are only administered topically or by injection to the penis);
22) Vasoactive intestinal protein (VIP), VIP mimetic, VIP analogue, more particularly mediated by one or more of the VIP receptor subtypes VPAC1, VPAC or PACAP (pituitory adenylate cyclase activating peptide), one or more of a VIP receptor agonist or a VIP analogue (e.g. Ro-125-1553) or a VIP fragment, one or more of a α-adrenoceptor antagonist with VIP combination (e.g. Invicorp, Aviptadil);
23) A melanocortin receptor agonist or modulator or melanocortin enhancer, such as melanotan II, PT-14, PT-141 or compounds claimed in WO-09964002, WO-00074679, WO-09955679, WO-00105401, WO-00058361, WO-00114879, WO-00113112, WO-09954358;
24) A serotonin receptor agonist, antagonist or modulator, more particularly agonists, antagonists or modulators for 5HT1A (including VML 670), 5HT2A, 5HT2C, 5HT3 and/or 5HT6 receptors, including those described in WO-09902159, WO-00002550 and/or WO-00028993;
25) A testosterone replacement agent (including dehydroandrostendione), testosternone (Tostrelle), dihydrotestosterone or a testosterone implant;
26) Estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone);
27) A modulator of transporters for noradrenaline, dopamine and/or serotonin, such as bupropion, GW-320659;
28) A purinergic receptor agonist and/or modulator;
29) A neurokinin (NK) receptor antagonist, including those described in WO-09964008;
30) An opioid receptor agonist, antagonist or modulator, preferably agonists for the ORL-1 receptor;
31) An agonist or modulator for oxytocin/vasopressin receptors, preferably a selective oxytocin agonist or modulator;
32) Modulators of cannabinoid receptors;
33) A SEP inhibitor (SEPi), for instance a SEPi having an IC₅₀ at less than 100 nanomolar, more preferably, at less than 50 nanomolar.
   Preferably, the SEP inhibitors according to the present invention have greater than 30-fold, more preferably greater than 50-fold selectivity for SEP over neutral endopeptidase NEP EC 3.4.24.11 and angiotensin converting enzyme (ACE). Preferably the SEPi also has a greater than 100-fold selectivity over endothelin converting enzyme (ECE).

By cross reference herein to compounds contained in patents and patent applications which can be used in accordance with invention, we mean the therapeutically active compounds as defined in the claims and the specific examples (all of which is incorporated herein by reference).

If a combination of active agents is administered, then they may be administered simultaneously, separately or sequentially.

Auxiliary Agents—PDE5 Inhibitors

The suitability of any particular cGMP PDE5 inhibitor can be readily determined by evaluation of its potency and selectivity using literature methods followed by evaluation of its toxicity, absorption, metabolism, pharmacokinetics; etc in accordance with standard pharmaceutical practice.

IC50 values for the cGMP PDE5 inhibitors may be determined using the PDE5 assay (see hereinbelow).

Preferably the cGMP PDE5 inhibitors used in the pharmaceutical combinations according to the present invention are selective for the PDE5 enzyme. Preferably (when used orally) they are selective over PDE3, more preferably over PDE3 and PDE4. Preferably (when oral), the cGMP PDE5 inhibitors of the invention have a selectivity ratio greater than 100 more preferably greater than 300, over PDE3 and more preferably over PDE3 and PDE4.

Selectivity ratios may readily be determined by the skilled person. IC50 values for the PDE3 and PDE4 enzyme may be determined using established literature methodology, see S A Ballard et al, Journal of Urology, 1998, vol. 159, pages 2164-2171 and as detailed herein after.

Suitable cGMP PDE5 inhibitors for the use according to the present invention include:
the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124. The pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclosed in EP-A-1092719.

Further suitable PDE5 inhibitors for the use according to the present invention include:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333); (+)-3-ethyl-5-[5-(4-ethylpiperazin-1-ylsulphonyl)-2-(2-methoxy-1 (R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d] pyrimidin-7-one (see WO99/54333); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl}-3-pyridylsulphonyl)-4-ethylpiperazine (see WO 01/27113, Example 8); 5-[2-iso-Butoxy-5-(4-ethylpiperazin-1-ylsulphonyl)pyridin-3-yl]-3-ethyl-2-(1-methylpiperdin-4-yl)-2,6-dihydro-7H-pyrazolo[4,3-d]pyrimidin-7-one (see WO 01/27113, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66); 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and the compound of example 11 of published international application WO93/07124 (EISAI); and compounds 3 and 14 from Rotella D P, J. Med. Chem., 2000, 43, 1257.

Still other suitable PDE5 inhibitors include:
4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

In vitro PDE inhibitory activities against cyclic guanosine 3′,5′-monophosphate (cGMP) and cyclic adenosine 3′,5′-monophosphate (cAMP) phosphodiesterases were determined by measurement of their IC₅₀ values (the concentration of compound required for 50% inhibition of enzyme activity).

The required PDE enzymes were isolated from a variety of sources, including human corpus cavernosum, human and rabbit platelets, human cardiac ventricle, human skeletal muscle and human and canine retina, essentially by the method of W. J. Thompson and M. M. Appleman (Biochem., 1971, 10, 311). In particular, the cGMP-specific PDE (PDE5) and the cGMP-inhibited cAMP PDE (PDE3) were obtained from human corpus cavernosum or human platelets; the cGMP-stimulated PDE (PDE2) was obtained from human corpus cavernosum and human platelets; the calcium/calmodulin (Ca/CAM)-dependent PDE (PDE1) from human cardiac ventricle; the cAMP-specific PDE (PDE4) from human skeletal muscle and human recombinant, expressed in SF9 cells; and the photoreceptor PDE (PDE6) from human or canine retina. Phosphodiesterases 7-11 were generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors was investigated by assaying a fixed amount of enzyme in the presence of varying inhibitor concentrations and low substrate, (cGMP or cAMP in a 3:1 ratio unlabelled to [³H]-labelled at a conc ˜1/3 Kₘ) such that IC₅₀≅Kᵢ. The final assay volume was made up to 100 μl with assay buffer [20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension (or in-house equivalent). Results from these tests show that the compounds of the present invention are inhibitors of cGMP-specific PDE5.

Functional activity can be assessed in vitro by determining the capacity of a compound of the invention to enhance sodium nitroprusside or electrical field stimulation-induced relaxation of pre-contracted rabbit corpus cavernosum tissue strips, using methods based on that described by S. A. Ballard et al. (Brit. J. Pharmacol., 1996, 118 (suppl.), abstract 153P) or S. A. Ballard et al. (J. Urology, 1998, 159, 2164-2171).

Compounds can be screened in vivo in test animals, such as anaesthetised rabbits, to determine their capacity, after i.v. administration, to enhance the pressure rises in the corpora cavernosa of the penis induced by intracavernosal injection of sodium nitroprusside, using a method based on that described by Trigo-Rocha et al. (Neurourol. and Urodyn., 1994, 13, 71).

Highly preferred for use in combination with a selective dopamine D3 receptor agonist in the pharmaceutical compositions herein are potent and selective PDE5 inhibitors.

Especially preferred herein is the combination of one or more potent and selective cGMP PDE5 inhibitors with one or more selective D3 dopamine receptor agonists.

Auxiliary Agents—SEP Inhibitors (SEPi)

A SEPi is a compound which inhibits or selectively inhibits a polypeptide having SEP activity.

SEP is a soluble secreted endopeptidase. Endopeptidases, including serine proteases, cysteine proteases and metalloendopeptidases, cleave at a sequence within an peptide.

An important group of endopeptidases known as zinc metalloproteases are characterised by having a requirement for the binding of a zinc ion in their catalytic site. Zinc metalloproteases can be subdivided into classes (for review see FEBS Letters 354 (1994) pp. 1-6), with one such class being the neprilysin (NEP)-like zinc metalloproteases (FASEB Journal, Vol 11, 1997 pp. 355-384). The NEP class includes at least 7 enzymes that are structurally related to each other (see later). They are typically membrane-bound, with a large carboxy-terminal extracellular domain, a short membrane-spanning region, and a short intracellular domain at the amino terminus. Known members of this family are neprilysin (also called NEP, CD10, CALLA, enkephalinase or EC 3.4.24.11), endothelin-converting enzymes (ECE-1 and ECE-2), PEX, KELL, X-converting enzyme/damage induced neural endopeptidase (XCE/DINE), and an enzyme identified in rodents called soluble secreted endopeptidase/neprilysin II (SEP/NEPII; Ghaddar, G et al, Biochem Journal, Vol 347, 2000, pp. 419-429; Ikeda, K et al, Journal Biological Chemistry, Vol 274, 1999, pp. 32469-32477; Tanja, O et al, Biochem Biophys Research Communication, Vol 271, 2000, pp. 565-570; International Patent Application WO 99/53077). The functions of the members of this class are thought to be related to peptidergic signalling. This is a process that occurs in most organisms, including humans, in which peptide molecules are used as “messengers” to elicit physiological responses. This typically involves the production and release of the peptide messenger by a specific cell, sometimes as an inactive precursor that is cleaved by a protease to become active. The active form of the peptide then binds a specific receptor on the surface of another cell where it elicits a response. The peptide is then inactivated by degradation by another protease.

NEPII is likely to be a rat equivalent of SEP, which is a mouse enzyme, as they share 91% amino acid identity. They are the members of this class closest to NEP in their amino acid sequence, both being 54% identical to human NEP. The mRNA of both is highly abundant in the testis and can also be detected at low levels in a broad range of other tissues. In the case of rat NEPII, the mRNA has also been found at comparatively high levels in the brain and pituitary. When produced recombinantly in mammalian cells, both mouse SEP and rat NEPII can be found in the growth media. This suggests they could be secreted proteases that may be able to circulate and hence cleave peptides at other sites in the body. Mouse SEP and rat NEPII, like some other members of this class such as ECE-1, exhibit splice variation. In the case of mouse SEP and rat NEPII, this splice variation results in isoforms with alterations in sequences involved in membrane localisation and secretion. The physiological significance of this is unclear but it is likely there could be membrane-bound, circulating, and intracellular forms of these enzymes. Mouse SEP has been shown to be able to cleave a range of important biological peptides including enkephalin, endothelin, big-endothelin, Bradykinin and substance P. Like NEP, therefore, it has a fairly broad substrate specificity and may have several physiological functions in different tissues.

Enzymes in this NEP class, like other metalloprotease enzymes, have been shown to be amenable to inhibition by small drug-like molecules (for example, thiorphan and phosphoramidon). This, together with the emerging nature of the physiological function of some members of the NEP-like enzymes in modulating peptidergic signalling, makes them attractive targets for pharmaceutical intervention.

Sequences for SEP are presented in WO99/53077, EP 1069188, WO02/06492 and WO00/47750 and also in SEQ ID NOS: 3-5 of the present application.

SEP sequences mentioned herein for, for example, assays, include references to any one or more of the sequences presented in WO99/53077, EP 1069188, WO02/06492 or WO00/47750 or presented as SEQ ID NO: 3, SEQ ID NO: 4 or SEQ ID NO: 5 or variants, fragments, homologues, analogues or derivatives thereof.

SEQ ID NO: 3 and SEQ ID NO: 4 each disclose a nucleotide sequence (cDNA) coding for human SEP. SEQ ID NO: 4 includes 5′ and 3′ partial vector sequences. SEQ ID NO: 5 shows a human SEP protein.

The suitability of any particular SEPi can be determined by evaluation of its potency and selectivity using, for example, the following assays followed by evaluation of its toxicity, absorption, metabolism, pharmacokinetics, etc. in accordance with standard pharmaceutical practice.

One SEP assay that may be used to detect candidate inhibitors of SEP is a fluorescence resonance energy transfer (FRET) assay. Most preferably, said labelled substrate peptide is Rhodamine green-Gly-Gly-dPhe-Leu-Arg-Arg-Val-Cys(QSY™-7)-βAla-NH₂.

SEP FRET Assay

The SEP FRET assay is based on an assay developed by Carvalho et al. for use with NEP (Carvalho et al., Annal. Biochem. 237, pp. 167-173 (1996)). The SEP FRET assay utilises a similar intramolecularly quenched fluorogenic peptide substrate, but with a novel combination of fluorogenic donor/acceptor dyes, specifically Rhodamine green (Molecular Probes, Inc., Eugene, Oreg., USA) and QSY™-7 (abbreviated hereafter as “QSY-7” or “QSY7”; Molecular Probes, Inc.).

The endopeptidase activity of SEP is measured by monitoring its ability to proteolyse the synthetic peptide substrate Rhodamine green-Gly-Gly-dPhe-Leu-Arg-Arg-Val-Cys(QSY7)-βAla-NH₂.

The two fluorophores (fluorogenic dyes) chosen for this assay have overlapping emission and absorption spectra and hence are suitable for energy transfer. The Rhodamine green acts as a donor and when excited at 485 nm gives out an emission (fluorescence) at 535 nm which in turn excites the QSY7 (FRET is occurring). The QSY7 is fluorescently silent and so gives off no emission above 535 nm hence no signal is observed (the Rhodamine green emission is quenched).

Upon cleavage (selective hydrolysis) by SEP at the Arg-Val peptide bond of the peptide substrate, the Rhodamine green and QSY7 moieties move apart and so upon excitation at 485 nm, energy transfer can no longer take place. As a result, an increase in fluorescence is observed at 535 nm for the Rhodamine green.

Preparation of the Synthetic Peptide Substrate Rhodamine green-Gly-Gly-dPhe-Leu-Arg-Arg-Val-Cys(QSY7)-βAla-NH₂

Peptide assembly was completed on 0.25 mmol FMOC-PAL-PEG-PS resin by solid phase peptide synthesis protocols using modifications to manufacturer supplied (Applied Biosystems, Foster City, Calif., USA) 9-fluoreneylmethoxycarbonyl (FMOC)-based synthesis cycles. Our modified cycles deprotect the amino terminus with 2×5 minute treatments with 20% piperidine/N-methylpyrrolidinone (NMP); the efficiency of which is monitored by UV absorbance at 301 nm by passage of a small aliquot of deprotection solution through a UV absorbance detector. In a separate cartridge, the incoming amino acid is activated with 0.9 equivalents each of 2-(1H-Benzotriazole-1-yl)-1,1,3,3 tetramethyluronium hexafluorophosphate (HBTU)/1-Hydroxybenzotriazole (HOBt) dissolved in N,N-dimethylformamide (DMF). 2 equivalents of diisopropylethyl amine (DIEA) are added. Concurrently, the resin is then washed with NMP to remove deprotection by-products. The wash solution is drained from the resin and the activated amino acid ester is transferred to the resin and stirred to allow coupling to the amino terminus for 20 minutes. The residual coupling solution is drained and the resin washed again with NMP. To ensure peptide homogeneity, a solution of 0.4M Acetic Anhydride/0.04M HOBt in NMP and 12 mmole DIEA are added to the resin to acetylate any potential unreacted sites. Finally, the resin is washed with NMP, drained, then washed with a mixture of 1:1 dichloromethane/2,2,2-trifluoroethanol and drained. This typifies one cycle of peptide synthesis. The completed synthesis resin was cleaved and deprotected using Reagent K (King, D. S. et. al., (1990), Int. J. Pep. Prot. Res., 36, pp. 255-66) affording 251 mg (100%) crude peptide CP1 Electrospray mass spectrometry (ESMS) (m/z calculation (calc.)=977.21 (MH+average), obs.=977.47).

Attachment of QSY-7 to Cysteine:

50 mg (51 μmol) of crude CP1 was dissolved in solution of 10% DIEA/DMF containing 45 mg (52.4 μmol) QSY-7 maleimide. After 10 minutes, the reaction was judged to be incomplete via HPLC-MS analysis and an additional 30 mg (30.7 μmol) crude peptide was added. After 30 additional minutes, the reaction was judged via HPLC-MS to be complete and all starting reagents consumed. The product was isolated by C18 preparative HPLC chromatography and fractions exhibiting desired product molecular weight by Matrix Assisted Laser Desorption Ionization mass spectrometry (MALDI-MS) were pooled and lyophilized to 73.7 mg (50%) of a purple powder, CP2 ESMS (m/z calc.=1797.86 (MH+monoisotopic), obs.=1797.86).

Attachment of bis(trifluoroacetyl) Rhodamine Green to the Amino Terminus:

73.7 mg (41 μmol) of CP2 was dissolved in a 2% DIEA/DMF solution containing 35 mg (52.8 μmol) Rhodamine Green carboxylic acid, trifluoroacetamide, succinimidyl ester (5(6)-CR 110 TFA, SE) *mixed isomers*. After 2 hours, the reaction was judged to be complete via HPLC-MS analysis. The product was isolated via C4 preparative HPLC chromatography and fractions exhibiting desired product molecular weights (MALDI-MS) were pooled and lyophilized to 71.4 mg (74%) of a purple powder CP3 ESMS (m/z calc.=2345.92 (MH+monoisotopic), obs.=2345.47).

Removal of Trifluoroacetyl Protecting Groups from Rhodamine Green:

71.4 mg (30.4 μmol) of CP3 was dissolved in 10 m[4:1 CH₃CN/H₂O. To this was added 200 mg (1886 μmol) Na₂CO₃. After 16 hr. vortexing, the supernatant was decanted from the insoluble material. The reaction vessel was rinsed with 1 ml DMSO; this was combined with the supernatant and the product isolated via C4 preparative HPLC chromatography. Fractions exhibiting product molecular weights (MALDI-MS) were combined and lyophilized to 64 mg (98%) of a purple powder, CP4 ESMS (m/z calc.=2155.54 (MH+average), obs.=2155.27). CP4 is the desired synthetic peptide substrate Rhodamine green-Gly-Gly-dPhe-Leu-Arg-Arg-Val-Cys(QSY7)-βAla-NH₂.

Materials:

All reagents were purchased of the highest commercial purity available and were used without further refinement. All reagents for peptide synthesis were purchased from Applied Biosystems, Foster City, Calif., USA with the following exceptions: QSY™-7 maleimide (Catalog number Q-10257) and Rhodamine Green carboxylic acid, trifluoroacetamide, succinimidyl ester (5(6)-CR 110 TFA, SE) *mixed isomers* (Catalog number R-6112) were purchased from Molecular Probes, Inc., OR, USA; FMOC-PAL-PEG-PS was purchased from Perseptive Biosystems, MA, USA (Catalog number GEN913384); FMOC-B-Alanine and FMOC-d-phenylalanine were purchased from Novabiochem, CA, USA; FMOC-Arg(Pbf)-OH was purchased from AnaSpec, Inc., CA, USA; 2,2,2-Trifluoroethanol was purchased from Aldrich, WI, USA. Sodium Carbonate was purchased from Fisher, PA, USA.

Preparative HPLC chromatography was performed on Vydac (CA, USA) C18 (Catalog number 218TP1022) or C4 (Catalog number 214TP1022) columns at 10 ml/min flow rate eluting with a linear gradient of 0% to 80% (A=5% CH₃CN/0.1% TFA/94.9% H₂O, B=100% CH₃CN) over 30 minutes collecting 30 second time fractions. Analytical HPLC-MS was performed using a Micromass (Manchester, UK) LCT mass spectrometer (masses based on externally calibrated standards) coupled with a Waters (MA, USA) 2690 HPLC inlet and a Waters 996 photodiode array detector performing chromatography on a Vydac C4 (Catalog number 214TP5415) column with a linear gradient of 0% to 80% (A=5% CH₃CN/0.1% TFA/94.9% H₂O, B=100% CH₃CN) over 30 minutes at 1 ml/min flow rate. Deconvoluted molecular weights were calculated from multiply charged observed ions using Micromass transform software. MALDI-MS were obtained on a Perseptive Biosystems Voyager-DE linear mass spectrometer using alpha cyano 4-hydroxy cinnamic acid matrix (Hewlett Packard, CA, USA) and reported masses based on external calibration.

Process (Including Chemical Structures):

CP4 (=synthetic peptide substrate Rhodamine green-Gly-Gly-dPhe-Leu-Arg-Arg-Val-Cys(QSY™-7)-βAla-NH₂) is synthesised by incorporating the key intermediate CP3 in a solid phase peptide synthesis scheme.

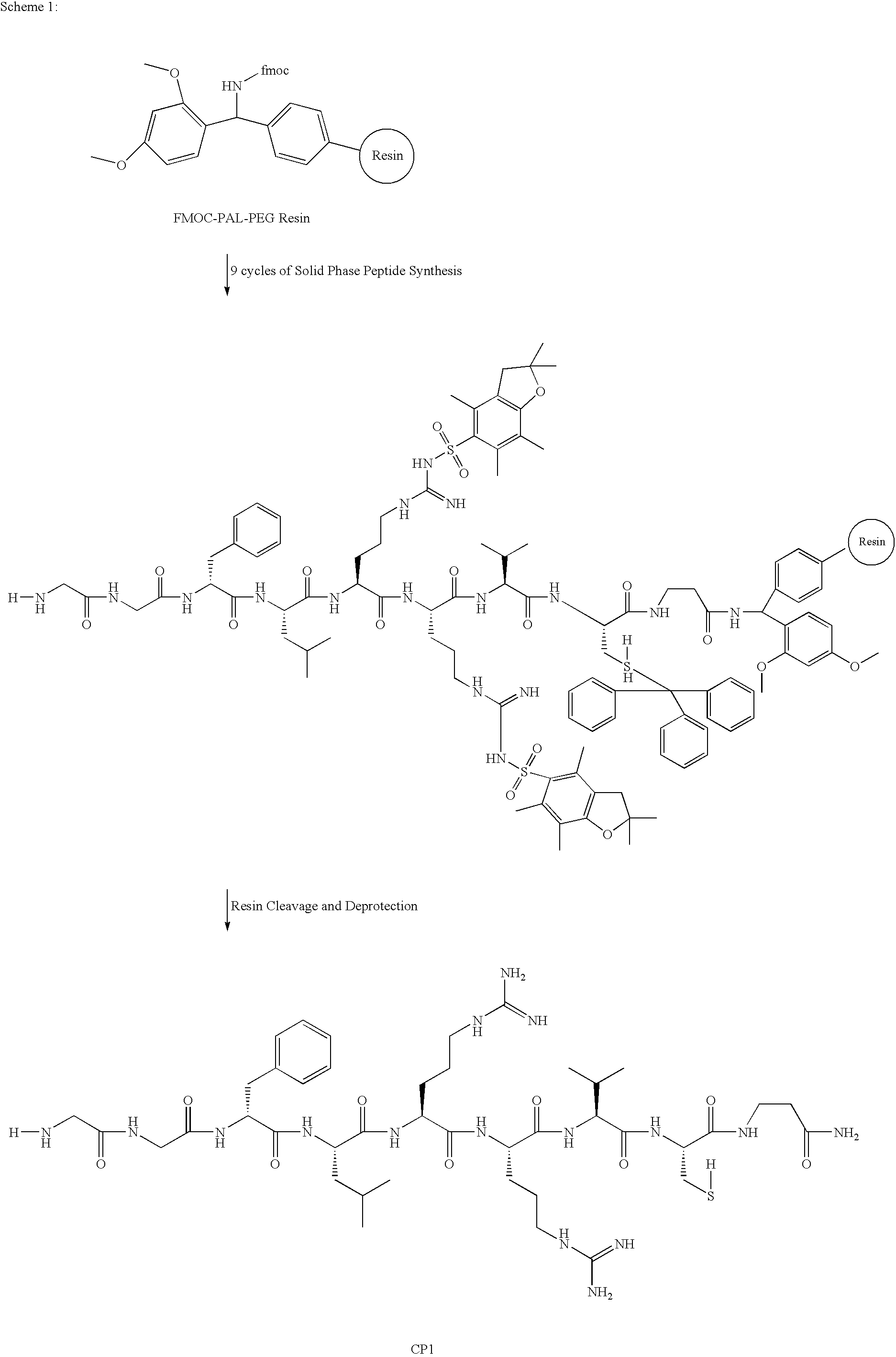

In summary, FMOC-PAL-PEG Resin is elaborated using Solid Phase Peptide Synthesis protocols optimised for efficiency of yield and time. These cycles (full details supra) incorporate 2 FMOC deprotections, washes, a single coupling of HBTU activated amino acid, washes, capping and finally, washing first with NMP then with 1:1 trifluoroethanol/dichloromethane. These washes help to relax resin secondary structure allowing for thorough deprotection and efficient coupling of the next incoming amino acid during the next cycle.

CP2 is synthesised (full details supra) according to Scheme 2:

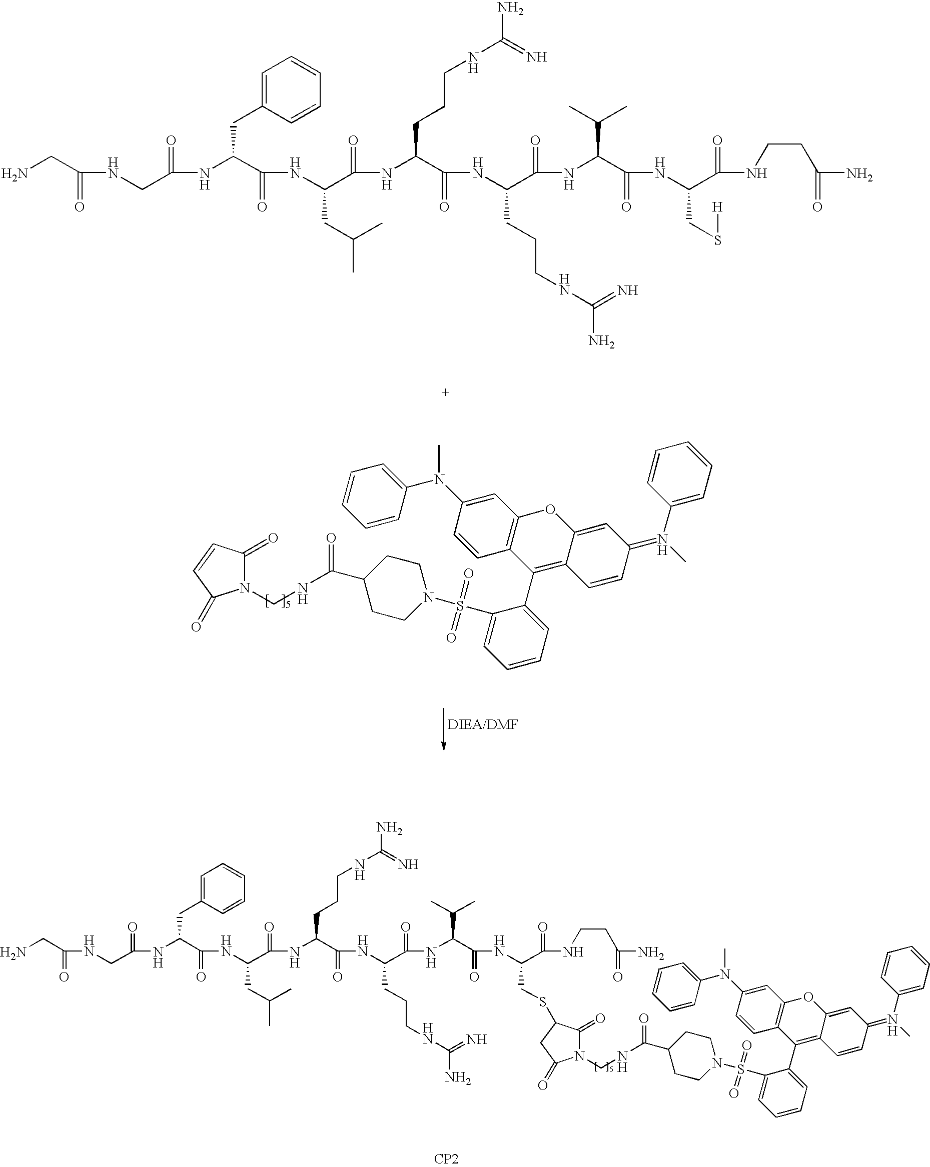

Following this incorporation of the QSY-7 tag, the second fluorophore, Rhodamine Green is added as the bis-trifluoroacetyl protected dye according to Scheme 3:

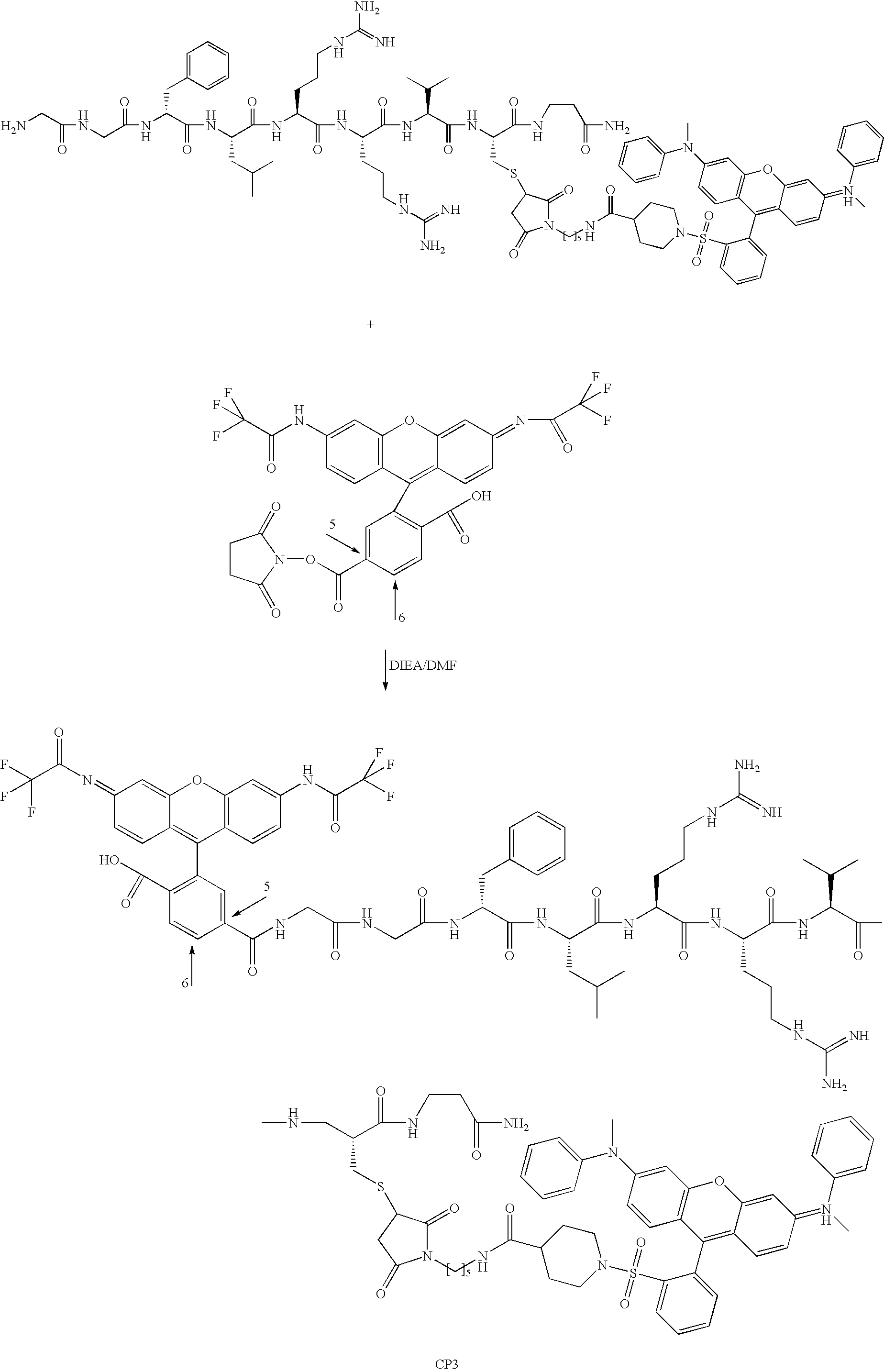

Finally, the trifluoroacetyl groups are removed by treatment with Na₂CO₃ affording the desired substrate, CP4: 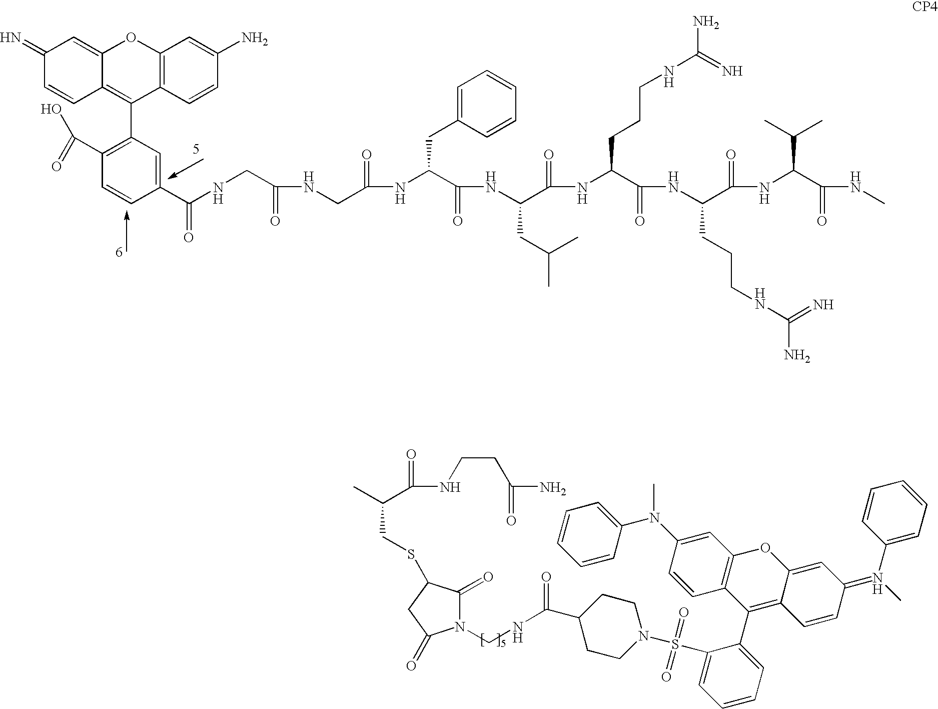
Assay

Reagents for the assay are first prepared as follows:

A substrate solution is made up by resuspending the substrate Rhodamine green-Gly-Gly-dPhe-Leu-Arg-Arg-Val-Cys(QSY7)-βAla-NH₂ in 50 mM HEPES buffer pH7.4 (Sigma, UK) at a concentration of 2 μM, then adding 1 EDTA-free protease inhibitor cocktail tablet (Roche Diagnostics, UK) per 25 ml.

An aliquot of SEP enzyme described above is thawed then diluted in 50 mM HEPES, pH7.4 by a predetermined factor specific to each enzyme batch, such that 50 μl contains sufficient enzyme to convert approximately 30% of substrate to product during the assay.

A 4% DMSO solution comprised of 4 ml DMSO plus 96 ml 50 mM HEPES pH7.4 is prepared.

A product solution is prepared by adding 500 μl of substrate solution to 250 μl enzyme solution plus 250 μl of 4% DMSO solution, and incubating at 37° C. for 16 hours.

Assays are set up as follows:

In a black 96 well microtitre plate, 100 μl of substrate solution is added to 50 μl of 4% DMSO solution. A similar non-specific background blank is also set up in which the 50 μl of 4% DMSO solution additionally contains 40 μM phosphoramidon. 50 μl of enzyme solution is added to the assay and blank, and the 96 well plate placed in a BMG galaxy fluorescence reader, operating with the Biolise software package (BMG Lab technologies, Offenberg, Germany).

The plate is incubated in the fluorescence reader for 1 hour at 37° C. and a fluorescence measurement taken every 3 minutes (Excitation (Ex) 485 nm/Emission (Em) 535 nm). The proteolytic activity of SEP corresponds to the rate of increase in fluorescence of the sample−rate of increase in fluorescence units of the non-specific background blank. The maximum velocity measurement (MaxV) calculated by the software over four successive readings is used for this calculation.

A fluorescence measurement taken from 200 μl of product in a well on an identical microtitre plate is taken. If required this value is used, together with the measured fluorescence units from the 60 min timepoint of the SEP assay, to calculate the percentage (%) of the substrate proteolysed during the 1 hour incubation period or to convert the measured rates of fluorescence increase into other useful units such as ng substrate proteolysed/min/ml enzyme.

The assay is used to calculate enzyme kinetic parameters such as Vmax and Km following standard principles described in Fundamentals of Enzyme Kinetics by Athel Cornish Bowden, 1979, published by Butterworths.

Using the SEP Assay to Determine the Inhibition Parameters of SEP Inhibitors

To determine the IC₅₀ of SEP inhibitors (for example phosphoramidon), multiple SEP assays are performed as described above with a range of test concentrations of inhibitor included in the 50 μl of DMSO solution (made by appropriate dilution of a 10 mM 100% DMSO stock of inhibitor with 4% DMSO/50 mM HEPES pH7.4). Using a suitable standard graph fitting computer program, a sigmoidal dose response curve is fitted to a plot of log inhibitor concentration versus MaxV (or % inhibition or % activity). The IC₅₀ is calculated as the inhibitor concentration causing 50% maximal inhibition. Typically for a given IC₅₀ determination, a dose range of at least 10 inhibitor concentrations differing in half log unit increments is used.

The SEP assay is used to determine the Ki and mode of inhibition (i.e. whether the inhibition is competitive, mixed, non-competitive, etc.) following standard enzymology principles as described, for example, in Fundamentals of Enzyme Kinetics by Athel Cornish Bowden, 1979, published by Butterworths.

NPY Inhibitors and/or NPY Y1 Inhibitors

Details of suitable assay systems for identifying and/or studying an NPYi (or an NPY Y1i) are presented hereinafter in the section entitled NPY assay and are based on the assay presented in WO-A-98/52890 (see page 96 thereof, lines 2 to 28).

Further examples of NPY inhibitors or NPY Y1 inhibitors are disclosed and discussed in the following review articles:
Dunlop J, Rosenzweig-Lipson S: Therapeutic approaches to obesity Exp Opin Ther Pat 1999 8 12 1683-1694
Wang S, Ferguson K C, Burris T P, Dhurandhar N V: 8th annual international conference on obesity and non-insulin dependent diabetes mellitus: novel drug developments. Exp Opin Invest Drugs 1999 8 7 1117-1125
Ling A L: Neuropeptide Y receptor antagonists Exp Opin Ther Pat 1999 9 4 375-384
Adham N, Tamm J, Du P, Hou C, et al: Identification of residues involved in the binding of the antagonist SNAP 6608 to the Y5 receptor Soc Neurosci Abstr 1998 24 part 2 626.9
Shu Y Z, Cutrone J Q, Klohr S E, Huang S: BMS-192548, a tetracyclic binding inhibitor of neuropeptide Y receptors, from Aspergillus niger WB2346. II. Physico-chemical properties and structural characterization J Antibiot 1995 48 10 1060-1065
Rigollier P, Rueger H, Whitebread S, Yamaguchi Y, Chiesi M, Schilling W, Criscione L: Synthesis and SAR of CGP 71683A, a potent and selective antagonist of the neuropeptide Y Y5 receptor. Int Symp Med Chem 1998 15th Edinburgh 239
Criscione L, Rigollier P, Batzl-Hartmann C, Rueger H, Stricker-Krongrad A, et al: Food intake in free-feeding and energy-deprived lean rats is mediated by the neuropeptide Y5 receptor. J Clin Invest 1998 102 12 2136-2145
Neurogen Corp: NGD 95-1 Clin Trials Monitor 1996 5 10 Ab 19244
Buttle L A: Anti-obesity drugs: on target for huge market sales. Exp Opin Invest Drugs 1996 5 12 1583-1587
Gehiert D R, Hipskind P A: Neuropeptide Y receptor antagonists in obesity. Exp Opin Invest Drugs 1996 7 9 1827-1838
Goldstein D J, Trautmann M E: Treatments for obesity. Emerging Drugs 1997 2-1-27
Hipskind P A, Lobb K L, Nixon J A, Britton T C, Bruns R F, Catlow J, Dieckman McGinty D K, Gackenheimer S L, Gitter B D, Iyengar S, Schober D A, et al.: Potent and selective 1,2,3-trisubstituted indole NPY Y-1 antagonists. J Med Chem 1997 40 3712-3714
Zimmerman D M, Cantrall B E, Smith E C R, Nixon J A, Bruns R F, Gitter B, Hipskind P A, Ornstein P L, Zarrinmayeh H, Britton T C, Schober D A, Gehiert D R: Structure-activity relationships of a series of 1-substituted-4-methylbenzimidazole neuropeptide Y-1 receptor antagonists Bioorganic Med Chem Lett 1998 8 5 473-476
Zarrinmayeh H, Nunes A, Ornstein P, Zimmerman D, Arnold M B, et al Synthesis and evaluation of a series of novel 2-[(4-chlorophenoxy)methy]benzimidazoles as selectiveneuropeptide Y Y1 receptor antagonists J Med Chem 1998 41 15 2709-2719
Britton T C, Spinazze P G, Hipskind P A, Zimmerman D M, Zarrinmayeh H, Schober D A, Gehlert D R, Bruns R F: Structure-activity relationships of a series of benzothiophene-dervied NPY-Y1 antagonists: optimization of the C2 side chain Bioorganic Med Chem Lett 1999 9 3 475-480
Zarrinmayeh H, Zimmerman D M, Cantrell B E, Schober D A, Bruns R F, Gackenheimer S L, Ornstein P L, Hipskind P A, Britton T C, Gehlert D R: Structure-activity relationship of a series of diaminoalkyl substituted benzimidazole as neuropeptide Y Y1 receptor antagonists Bioorganic Med Chem Lett 1999 9 5 647-652
Murakami Y, Hara H, Okada T, Hashizume H, Kii M, Ishihara Y, Ishikawa M, Mihara S-I, Kato G, Hanasaki K, Hagishita S, Fujimoto M: 1,3-disubstituted benzazepines as novel, potent, selective neuropeptide Y Y1 receptor antagonists J Med Chem 1999 42 14 2621-2632
Rudolf K, Eberlein W, Engel W, Wieland H A, Willim K D, Entzeroth M, Wienen W, Beck Sickinger A G, Doods H N: The first highly potent and selective non-peptide neuropeptide YY1 receptor antagonist: BIBP3226 Eur J Pharmacol 1994 271 2-3 R11-R13
Wieland H A, Willim K D, Entzeroth M, Wienen W, Rudolf K, Eberlein W, Engel W, Doods H N: Subtype selectivity and antagonist profile of the nonpeptide neuropeptide Y1 receptor antagonist BIBP 3226 J Pharmacol Exp Ther 1995 275 1 143-149.
Wright J, Bolton G, Creswell M, Downing D, Georgic L, Heffner T, Hodges J, MacKenzie R, Wise L: 8-amino-6-(arylsulphonyl)-5-nitroquinolones: novel nonpeptide neuropeptide Y1 receptor antagonists Bioorganic Med Chem Lett 1996 6 15 1809-1814
Capurro D, Huidobro-Toro J P: The involvement of neuropeptide Y Y1 receptors in the blood pressure baroreflex:studies with BIBP 3226 and BIB 3304. Eur J Pharmacol 1999 376 3 251-255
Dumont Y, Cadieux A, Doods H, Quirion R: New tools to investigate neuropeptide Y receptors in the central and peripheral nervous systems: BIBO-3304 (Y1), BIIE-246 (Y2) and [125I]-GR-231118 (Y1/Y4). Soc Neurosci Abstr 1999 25 Part 1 Abs 74.11
Hegde S S, Bonhaus D W, Stanley W, Eglen R M, Moy T M, Loeb M, et al Pharmacological evaluation of 1229U91, a high affinity and selective neuropeptide Y(NPY)-Y1 receptor antagonist Pharmacol Res 1995 31 190
Matthews J E, Chance W T, Grizzle M K, Heyer D, Daniels A J: Food intake inhibition and body weight loss in rats treated with GI 264879A, an NPY-Y1 receptor. Soc Neurosci Abstr 1997 23 Pt 2 1346
Doods H N, Willim K-D, Smith S J: BIBP 3226: a selective and highly potent NPY-Y1 antagonist Proc Br Pharmacol Soc 1994 13-16 Dec. C47
Rudolf K, Eberlein W, Engel W, Wieland H A, Willim K D, Entzeroth M, Wienen W, Beck Sickinger A G, Doods H N: The first highly potent and selective non-peptide neuropeptide YY1 receptor antagonist: BIBP3226 Eur J Pharmacol 1994 271 2-3 R11-R13
Serradelil-Le-Gal C, Valette G, Rouby P E, Pellet A, Villanova G, Foulon L, Lespy L, Neliat G, Chambon J P, Maffrand J P, Le-Fur G: SR 120107A and SR 120819A: Two potent and selective, orally-effective antagonists for NPY Y1 receptors Soc Neurosci Abstr 1994 20 Pt 1 907-Abs 376.14
Hong Y, Gregor V, Ling A L, Tompkins E V, Porter J, Chou T S, Paderes G, Peng Z, Hagaman C, Anderes K, Luthin D, May J: Synthesis and biological evaluation of novel guanylurea compounds as potent NPY Y1 receptor antagonist Acs 1999 217 Anaheim MEDI 108

Yet further examples of NPYi's and/or NPY Y1 i's are disclosed in the following documents:
WO-98/07420
WO-94/00486
WO-96/22305
WO-97/20821
WO-97/20822
WO-96/14307
JP-07267988
WO-96/12489
U.S. Pat. No. 5,552,422
WO-98/35957
WO-96/14307
WO-94/17035
EP-0614911
WO-98/40356
EP-0448765
EP-0747356
WO-98/35941
WO-97/46250
EP-0747357
EP-0896822
EP-1033366
WO-00/66578

Further examples of NPY inhibitors and/or NPY Y1 inhibitors are selected from the following structures:

| | | Mode of Action |
|---|---|---|
| Compound | Structure | References |
| F34 | 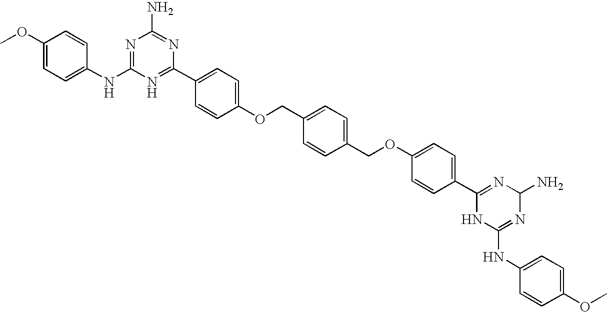 | I:NPY Y1 WO-98/07420 |
| F35 | 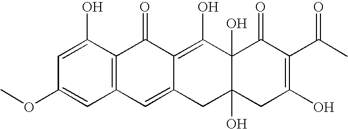 | I:NPY |
| F37 | Ile-Cys-Pro- | I:NPY Y1 |
| | Cys-Tyr-Arg-Leu-Arg-Tyr- | WO-94/00486 |
| | NH2 cyclic (2,2′), (4,4′)-disulfide | WO-96/22305 |
| | dimer | |
| F39 | 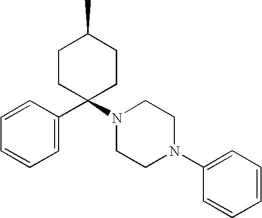 | I:NPY Y1 WO-96/14307 |
| F40 | 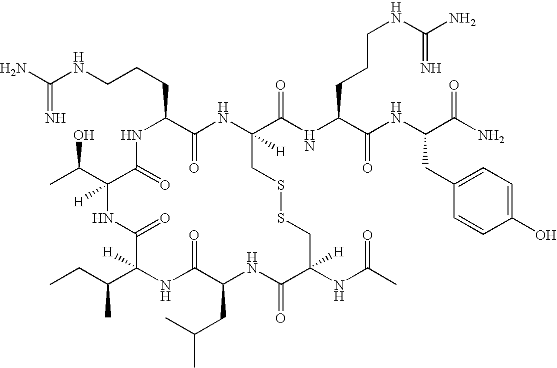 | I:NPY Y1 JP-07267988 |
| F41 | 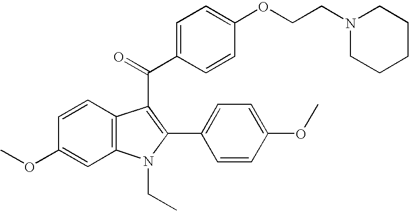 | I:NPY Y1 WO-96/12489 |
| F42 | 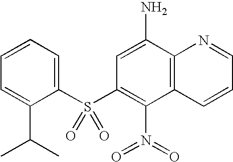 | I:NPY Y1 US-5552422 |
| F44 | 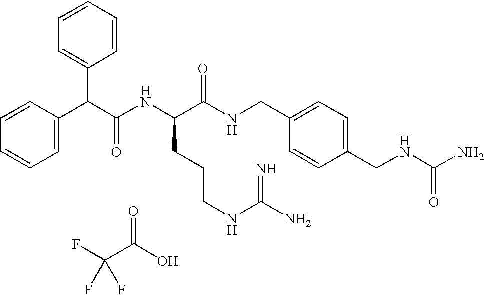 | I:NPY Y1 |
| F45 | 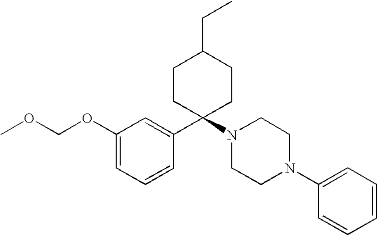 | I:NPY Y1 WO-96/14307 |
| F47a | 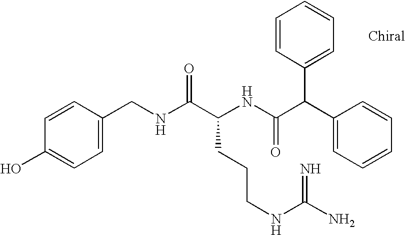 | I:NPY Y1 WO-94/17035 (BIBP 3226) |
| F48 | 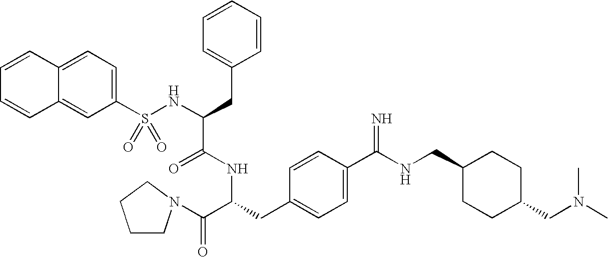 | I:NPY Y1 EP-0614911 |
| F49 | 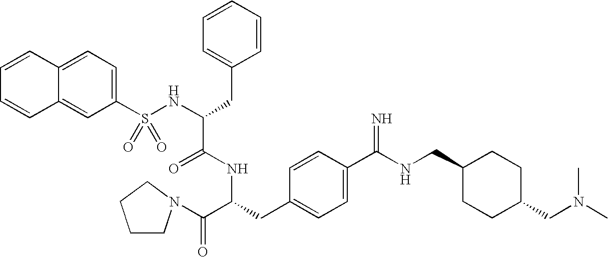 | I:NPY Y1 EP-0614911 |
| F50 | 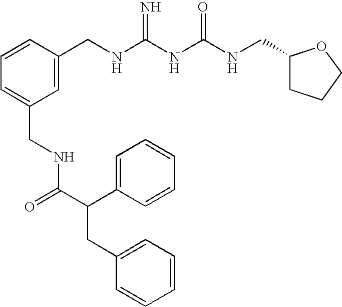 | I:NPYY1 |
| F52 | 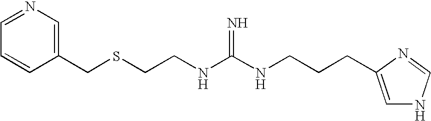 | I:NPY EP-0448765 |
| F53 | 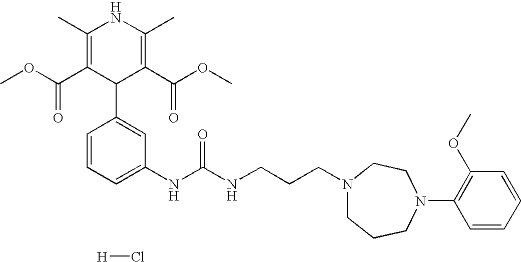 | I:NPY Y1 EP-0747356 |
| F54 | 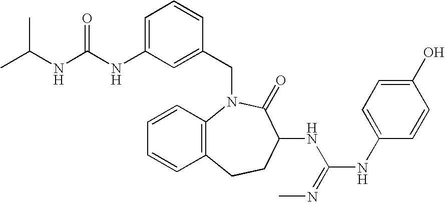 | I:NPY Y1 WO-98/35941 |
| F56 | 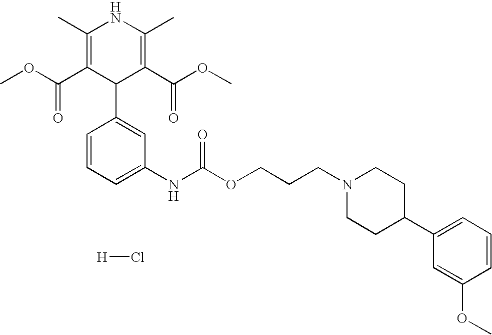 | I:NPY Y1 EP-0747357 |
| F57 | 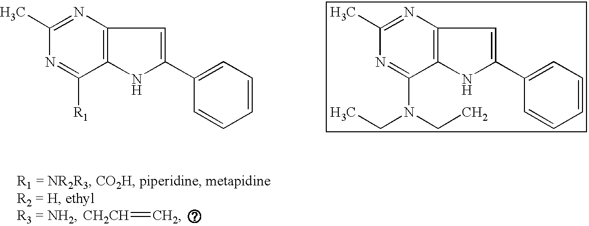 | I:NPY EP-0896822 |
| F58 | 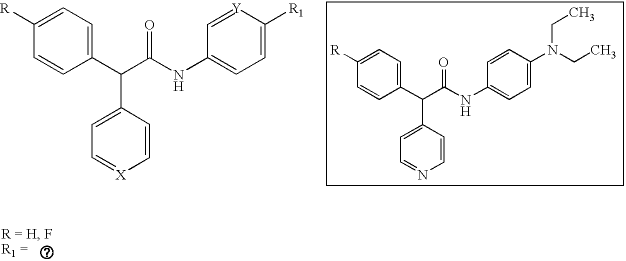 | I:NPY EP-1033366 |
| F59 | 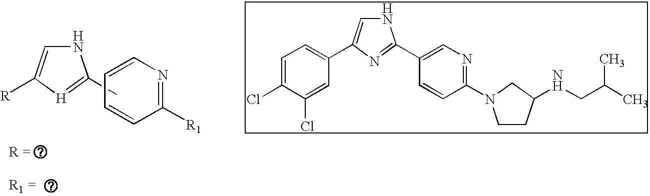 | I:NPY WO-00/66578 |

NPY Assays

As taught in WO98/52890 (page 96, lines 2-28), the ability of compounds to bind to NPY may be assessed using a protocol essentially as described in M. W. Walker et al Journal of Neurosciences 8:2438-2446 (1988).

In this assay the cell line SK-N-MC was employed. This cell line was available from Sloane-Kettering Memorial Hospital, New York.

These cells were cultured in T-150 flasks using Dulbecco's Minimal Essential Media (DMEM) supplemented with 5% fetal calf serum. The cells were manually removed from the flasks by scraping, pelleted, and stored at −70° C.

The pellets were resuspended using a glass homogeniser in 25 mM HEPES (pH7.4) buffer containing 2.5 mM calcium chloride, 1 mM magnesium chloride and 2 g/L bacitracin. Incubations were performed in a final volume of 200 μl containing 0.1 nM ¹²⁵I-peptide YY (2200 Ci/mmol) and 0.2-0.4 mg protein for about two hours at room temperature.

Nonspecific binding was defined as the amount of radioactivity remaining bound to the tissue after incubating in the presence of 1 μM neuropeptide Y. In some experiments various concentrations of compounds were included in the incubation mixture.

Incubations were terminated by rapid filtration through glass fibre filters which had been presoaked in 0.3% polyethyleneimine using a 96-well harvester. The filters were washed with 5 ml of 50 mM Tris (pH7.4) at 4° C. and rapidly dried at 60° C. The filters were then treated with melt-on scintillation sheets and the radioactivity retained on the filters were counted. The results were analysed using various software packages. Protein concentrations were measured using standard coumassie protein assay reagents using bovine serum albumin as standards.

NEP Inhibitors (I:NEP═NEPi)

NEP EC3.4.24.11 (FEBS Lett. 229(1), 206-210 (1988)), also known as enkephalinase or neprilysin, is a zinc-dependent neutral endopeptidase. This enzyme is involved in the breakdown of several bioactive oligopeptides, cleaving peptide bonds on the amino side of hydrophobic amino acid residues. The key neuronally released bioactive agents or neuropeptides metabolised by NEP include natriuretic peptides such as atrial natriuretic peptides (ANP) as well as brain natriuretic peptide and C-type natriuretic peptide, bombesin, bradykinin, calcitonin gene-related peptide, endothelins, enkephalins, neurotensin, substance P and vasoactive intestinal peptide. Some of these peptides have potent vasodilatory and neurohormone functions, diuretic and natriuretic activity or mediate behaviour effects. Background teachings on NEP have been presented by Victor A. McKusick et al on http://www3.ncbi.nlm.nih.gov/Omim/searchomim.htm.

The suitability of any particular I:NEP can be readily determined by evaluation of its potency and selectivity using literature methods followed by evaluation of its toxicity, absorption, metabolism, pharmacokinetics, etc in accordance with standard pharmaceutical practice.

Preferably the I:NEP have a selectivity over ACE of greater than 300.

IC₅₀ values and selectivity ratios for ACE may be determined by methods described in EP1097719A1.

Examples of NEP inhibitors are disclosed and discussed in the following review articles: Pathol. Biol., 46(3), 1998, 191; Current Pharm. Design, 2(5), 1996, 443; Biochem. Soc. Trans., 21(3), 1993, 678; Handbook Exp. Pharmacol., 104/1, 1993, 547; TiPS, 11, 1990, 245; Pharmacol. Rev., 45(1), 1993, 87; Curr. Opin. Inves. Drugs, 2(11), 1993, 1175; Antihypertens. Drugs, (1997), 113; Chemtracts, (1997), 10(11), 804; Zinc Metalloproteases Health Dis. (1996), 105; Cardiovasc. Drug Rev., (1996), 14(2), 166; Gen. Pharmacol., (1996), 27(4), 581; Cardiovasc. Drug Rev., (1994), 12(4), 271; Clin. Exp. Pharmacol. Physiol., (1995), 22(1), 63; Cardiovasc. Drug Rev., (1991), 9(3), 285; Exp. Opin. Ther. Patents (1996), 6(11), 1147.

Further examples of NEP inhibitors are disclosed in the following documents: EP-509442A; US-192435; U.S. Pat. No. 4,929,641; EP-599444B; US-884664; EP-544620A; US-798684; J. Med. Chem. 1993, 3821; Circulation 1993, 88(4), 1; EP-136883; JP-85136554; U.S. Pat. No. 4,722,810; Curr. Pharm. Design, 1996, 2, 443; EP-640594; J. Med. Chem. 1993, 36(1), 87; EP-738711-A; JP-270957; CAS # 115406-23-0; DE-19510566; DE-19638020; EP-830863; JP-98101565; EP-733642; WO9614293; JP-08245609; JP-96245609; WO9415908; JP05092948; WO-9309101; WO-9109840; EP-519738; EP-690070; J. Med. Chem. (1993), 36, 2420; JP-95157459; Bioorg. Med. Chem. Letts., 1996, 6(1), 65; EP-A-0274234; JP-88165353; Biochem. Biophys. Res. Comm., 1989, 164, 58; EP-629627-A; US-77978; Perspect. Med. Chem. (1993), 45; EP-358398-B

Further examples of NEP inhibitors are disclosed in EP1097719-A1, in particular compounds FXII to FXIII therein.

Preferred NEP inhibitors are compounds FV to FX1 and F57 to F65 of EP1097719-A1.

Bombesin Receptor Antagonists

Compounds that are bombesin receptor antagonists have been tested using animal models that are believed to be reliable and predictive, in particular with the capacity to make predictions for females. In rodents proceptive behaviour is under hormonal control, progesterone being essential for induction of proceptive behaviour in combination with oestrogen (Johnson M and Everitt B., Essential Reproduction (3^{rd} edn), Blackwell, Oxford, 1988). The evidence for the hormonal control of proceptive behaviour in primates is conflicting, but on the whole oestrogens and/or androgens appear to enhance proceptive behaviour (Baum M. J., J. Biosci., 1983; 33:578-582). The behavioural manifestations of proceptive behaviour in the rat include “hopping and darting” movement, with rapid vibration of the ears. Tests to assess the eagerness to seek sexual contact (sexual motivation) have been reported as the most appropriate way to measure proceptivity (Meyerson B. J, Lindstrom L. H., Acta Physiol. Scand., 1973; 389 (Suppl.): 1-80). Receptivity, in the rat, is demonstrated when the female assumes a lordotic position. This occurs when, on mounting, the male exerts pressure with his forepaws on the flanks of the receptive female. The main sites of neuronal control for this behaviour are the ventromedial nucleus (VMN) and the midbrain central grey area (MCG) (for review, see Wilson C. A., In: Sexual Pharmacology, Riley A. J. et al, (Eds), Clarendon Press, Oxford, 1993: 1-58).

Bombesin is a 14-amino acid peptide originally isolated from the skin of the European frog Bombina bombina (Anastasi A. et al., Experientia, 1971; 27: 166). It belongs to a class of peptides which share structural homology in their C-terminal decapeptide region (Dutta A. S., Small Peptides; Chemistry, Biology, and Clinical Studies, Chapter 2, pp 66-82). At present, two mammalian bombesin-like peptides have been identified, the decapeptide neuromedin B (NMB) and a 23-residue amino acid, gastrin-releasing peptide (GRP).

Bombesin evokes a number of central effects through actions at a heterogeneous population of receptors. The BB₁ receptor binds neuromedin B (NMB) with higher affinity than gastrin-related peptide (GRP) and neuromedin C (NMC) and BB₂ receptors bind GRP and NMC with greater affinity than NMB. More recently evidence has emerged of two more receptor subtypes denoted BB₃ and BB₄ but due to limited pharmacology, little is known of their function at present. BB₁ and BB₂ receptors have a heterogeneous distribution within the central nervous system indicating that the endogenous ligands for these receptors may differentially modulate neurotransmission. Among other areas, BB₁ receptors are present in the ventromedial hypothalamus (Ladenheim E. E et al, Brain Res., 1990; 537: 233-240).

Bombesin-like immunoreactivity and mRNA have been detected in mammalian brain (Braun M., et al., Life. Sci., 1978; 23: 2721) (Battey J., et al., TINS, 1991; 14:524). NMB and GRP are believed to mediate a variety of biological actions (for a review, see WO 98/07718).

The following patent applications disclose compounds capable of antagonising the effects of NMB and/or GRP at bombesin receptors: CA 2030212, EP 0309297, EP 0315367, EP 0339193, EP 0345990, EP 0402852, EP 0428700, EP 0438519, EP 0468497, EP 0559756, EP 0737691, EP 0835662, JP 07258081, UK 2231051, U.S. Pat. No. 4,943,561, U.S. Pat. No. 5,019,647, U.S. Pat. No. 5,028,692, U.S. Pat. No. 5,047,502, U.S. Pat. No. 5,068,222, U.S. Pat. No. 5,084,555, U.S. Pat. No. 5,162,497, U.S. Pat. No. 5,244,883, U.S. Pat. No. 5,439,884, U.S. Pat. No. 5,620,955, U.S. Pat. No. 5,620,959, U.S. Pat. No. 5,650,395, U.S. Pat. No. 5,723,578, U.S. Pat. No. 5,750,646, U.S. Pat. No. 5,767,236, U.S. Pat. No. 5,877,277, U.S. Pat. No. 5,985,834, WO 88/07551, WO 89/02897, WO 89/09232, WO 90/01037, WO 90/03980, WO 91/02746, WO 91/04040, WO 91/06563, WO 92/02545, WO 92/07830, WO 92/09626, WO 92/20363, WO 92/20707, WO 93/16105, WO 94/02018, WO 94/02163, WO 94/21674, WO 95/00542, WO 96/17617, WO 96/28214, WO 97/09347, WO 98/07718, WO 00/09115, WO 00/09116. We believe that compounds disclosed in these applications can be used in the prevention or treatment of sexual dysfunction, which is an indication that is not disclosed or suggested by the aforesaid applications, or indeed in any previous scientific publication concerning bombesin receptors.

One preferred genus of bombesin receptor antagonists disclosed in WO 98/07718 comprises compounds of the formula (I) 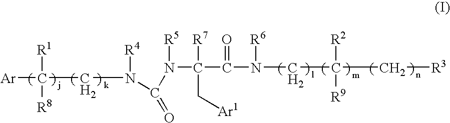
and pharmaceutically acceptable salts thereof, wherein:
j is 0 or 1;
k is 0 or 1;
l is 0, 1, 2, or 3;
m is 0 or 1;
n is 0, 1 or 2;
Ar is phenyl, pyridyl or pyrimidyl, each unsubstituted or substituted by from 1 to 3 substituents selected from alkyl, halogen, alkoxy, acetyl, nitro, amino, —CH₂NR¹⁰R¹¹, cyano, —CF₃, —NHCONH₂, and —CO₂R¹²;
R¹ is hydrogen or straight, branched, or cyclic alkyl of from 1 to 7 carbon atoms;
R⁸ is hydrogen or forms a ring with R¹ of from 3 to 7 carbon atoms;
R² is hydrogen or straight, branched, or cyclic alkyl of from 1 to 8 carbon atoms which can also contain 1 to 2 oxygen or nitrogen atoms;
R⁹ is hydrogen or forms with R² a ring of from 3 to 7 carbon atoms which can contain an oxygen or nitrogen atom; or R² and R⁹ can together be a carbonyl;
Ar¹ can be independently selected from Ar and can also include pyridyl-N-oxide, indolyl, imidazolyl, and pyridyl;
R⁴, R⁵, R⁶, and R⁷ are each independently selected from hydrogen and lower alkyl; R⁴ can also form with R⁵ a covalent link of 2 to 3 atoms which may include an oxygen or a nitrogen atom;
R³ can be independently selected from Ar or is hydrogen, hydroxy, —NMe₂, N-methyl-pyrrolyl, imidazolyl, N-methyl-imidazolyl, tetrazolyl, N-methyl-tetrazolyl, thiazolyl, —CONR¹³R¹⁴, alkoxy, 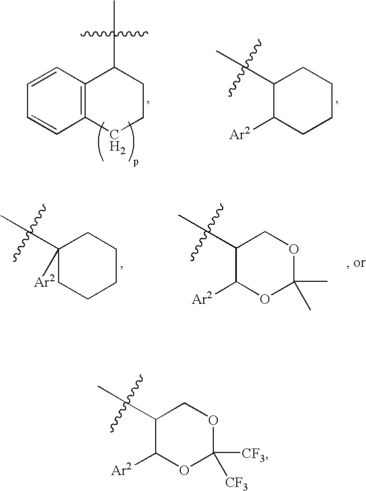
wherein p is 0, 1 or 2 and Ar² is phenyl or pyridyl;
R¹⁰, R¹¹, R¹², R¹³ and R¹⁴ are each independently selected from hydrogen or straight, branched, or cyclic alkyl of from 1 to 7 carbon atoms.

A particularly preferred compound within the above genus is (S) 3-(1H-Indol-3-yl)-N-[1-(5-methoxy-pyridin-2-yl)-cyclohexylmethyl]-2-methyl-2-[3-(4-nitro-phenyl)-ureido]-propionamide and its pharmaceutically acceptable salts.

BB₁ and BB₂ Binding Assays

In the following experiments, measurement of BB₁ and BB₂ binding was as follows. CHO-K1 cells stably expressing cloned human NMB (for (BB₁ assay) and GRP receptors (for BB₂ assay) were routinely grown in Ham's F12 culture medium supplemented with 10% foetal calf serum and 2 mM glutamine. For binding experiments, cells were harvested by trypsinization, and stored frozen at −70° C. in Ham's F12 culture medium containing 5% DMSO until required. On the day of use, cells were thawed rapidly, diluted with an excess of culture medium, and centrifuged for 5 minutes at 2000 g. Cells were resuspended in 50 mM Tris-HCl assay buffer (pH 7.4 at 21° C., containing 0.02% BSA, 40 μg/mL bacitracin, 2 μg/mL chymostatin, 4 μg/mL leupeptin, and 2 μM phosphoramidon), counted, and polytronned (setting 5, 10 sec) before centrifuging for 10 minutes at 28,000 g. The final pellet was resuspended in assay buffer to a final cell concentration of 1.5×10⁵/mL. For binding assays, 200 μL aliquots of membranes were incubated with [¹²⁵I][Tyr⁴]bombesin (<0.1 nM) in the presence and absence of test compounds (final assay volume 250 μL) for 60 minutes and 90 minutes for NMB and GRP receptors, respectively. Nonspecific binding was defined by 1 μM bombesin. Assays were terminated by rapid filtration under vacuum onto Whatman GF/C filters presoaked in 0.2% PEI for >2 hours, and washed 50 mM Tris-HCl (pH 6.9 at 21° C.; 6×1 mL). Radioactivity bound was determined using a gamma counter.

All competition data was analysed using nonlinear regression utilizing iterative curve-plotting procedures in Prism® (GraphPad Software Inc., San Diego, USA). IC₅₀ values were corrected to Ki values using the Cheng-Prusoff equation (Cheng Y., Prusoff W. H., Biochem. Pharmacol. 22: 3099-3108, 1973).

Modulators of Intermediate Conductance Calcium-Activated Potassium (IK_{Ca}) Channels

The term “calcium-activated potassium channels” includes large conductance calcium activated (BK_{Ca}) channels (also referred to as Maxi K⁺ channels), small conductance calcium activated (SK_{Ca}) channels and intermediate conductance calcium activated (IK_{Ca}) channels which are sometimes referred to as an hSK₄ channels or IK channels or hIK₁ channels.

Currently there are three subtypes of calcium-activated potassium channels. These are large conductance calcium activated (BK_{Ca}) channels, intermediate conductance calcium activated (IK_{Ca}) channels and small conductance calcium activated (SK_{Ca}) channels. These channels are characterised by the degree of ionic conductance that passes through the channel pore during a single opening (Fan et al 1995). By way of distinction: large conductance (BK) channels are gated by the concerted actions of internal calcium ions and membrane potential and have a unit conductance of 100 to 220 picoSiemens (pS); whereas Intermediate conductance (IK) and small conductance (SK) channels are gated solely by internal calcium ions. By way of further distinction, the IK_{Ca} and SK_{Ca} channels have a unit conductance of 20 to 85 pS and 2 to 20 pS, respectively, and are more sensitive to calcium than are BK channels. Each type of channel shows a distinct pharmacology (Ishii et al 1997).

As used herein, the term “intermediate conductance calcium activated (IK_{Ca}) channel” refers to a subtype of the calcium activated potassium channels which is characterised by the degree of ionic conductance that passes through the channel pore during a single opening (Fan et al 1995). In contrast to the large conductance (BK) channels which are gated by the concerted actions of internal calcium ions and membrane potential and have a unit conductance of 100 to 220 picoSiemens (pS), the intermediate conductance (IK) channel is gated solely by internal calcium ions, with a unit conductance of 20 to 85 pS and is more sensitive to calcium than the BK channels.

As used herein the term “modulating IK_{Ca} channel activity” means any one or more of: improving, increasing, enhancing, agonising, depolarising or upregulating IK_{Ca} channel activity or that the Ca²⁺ sensitivity of the IK_{Ca} channel is increased—that is, the calcium concentration required to elicit IK_{Ca} channel activity/opening is lowered. The increase in the Ca²⁺ sensitivity of the IK_{Ca} channel may be increased/enhanced by a direct or indirect opening of the IK_{Ca} channels. This increase in the Ca²⁺ sensitivity of the IK_{Ca} channel may result in a modification of the IK_{Ca} channel characteristics such that the IK_{Ca} channel opening is affected in such a way that the IK_{Ca} channel opens earlier and/or at lower intracellular calcium concentrations and/or for longer periods of time and/or with an increased open time probability.

The term “modulating IK_{Ca} channel activity” also includes the upregulation of IK_{Ca} channel expression in corpus cavernosum smooth muscle tissue such as, for example, by an agent that increases the expression of the IK_{Ca} channel and/or by the action of an agent on a substance that would otherwise impair and/or antagonise the modulation of IK_{Ca} channel activity and/or the expression of the IK_{Ca} channel.

By way of example the modulator may have the structure of formula (I): 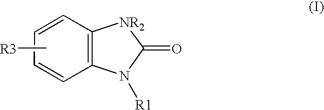
wherein:
R1 is a H or a suitable substituent, such as an alkyl group which may be optionally substituted;
R2 is a H or a suitable substituent, preferably H
R3 represents one or more suitable optional substituents.

Alternatively, the modulator may have the structure of formula (1): 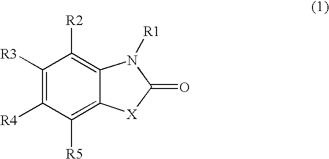
wherein:
X is selected from NR, O or S
   wherein R is H or alkyl (preferably lower alkyl, more preferably C1-6 alkyl)
R1 is alkyl (preferably lower alkyl, more preferably C1-6 alkyl)
R2 is selected from H, halide, alkyl (preferably lower alkyl, more preferably C1-6 alkyl), alkoxy (preferably lower alkoxy, more preferably C1-6 alkoxy)
R3 is selected from H, halide, alkyl (preferably lower alkyl, more preferably C1-6 alkyl), alkoxy (preferably lower alkoxy, more preferably C1-6 alkoxy)
R4 is selected from H, halide, alkyl (preferably lower alkyl, more preferably C1-6 alkyl), alkoxy (preferably lower alkoxy, more preferably C1-6 alkoxy)
R5 is selected from H, halide, alkyl (preferably lower alkyl, more preferably C1-6 alkyl), alkoxy (preferably lower alkoxy, more preferably C1-6 alkoxy).

Compounds of formula (1)—wherein X=O (formula (1a)) or wherein X=S (formula (1b))—can be prepared by N-alkylation under basic conditions of the respective corresponding parent heterocycles (2a) or (2b), these in turn may be prepared by the treatment of the respective corresponding aminophenol (3a) or aminothiophenol (3b) with phosgene or another suitable carbonylating agent. Aminophenols and aminothiophenols are usually prepared from the respective corresponding nitrophenols (4a) or nitrothiophenols (4b) by reduction. Many substituted nitrophenols (4a) and nitrothiophenols (4b) are commercially available.

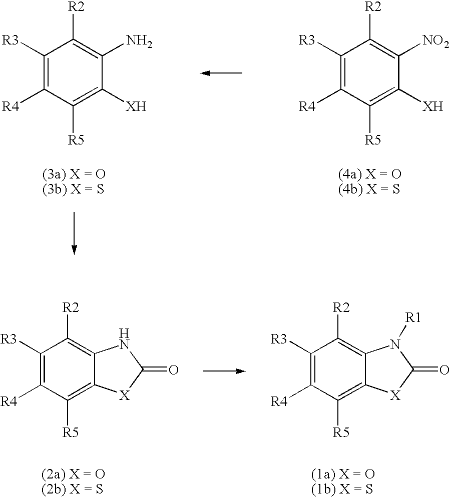

Compounds of formula 1 where X=NH (formula (1c)) can be prepared by a modification to the above scheme. In this respect, alkylation of a respective corresponding nitroaniline (5c) is carried out prior to reduction of the nitro group, providing a phenyldiamine (3c, X=NH) that is cyclised to 1c by carbonylation as described above.

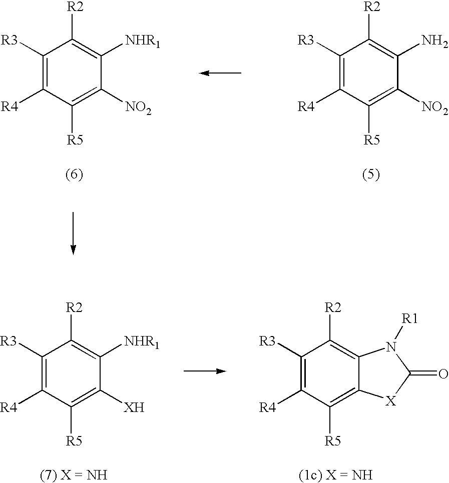

Preferably the modulator is EBIO (1-ethyl-2-benzimidazolinone) or a mimetic thereof or a pharmaceutically acceptable salt of any thereof. The structure of EBIO is:

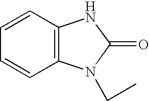

For some applications, preferably the agent has an IC₅₀ value of less than 300 nM, 250 nM, 200 nM, 150 nM, preferably less than about 100 nM, preferably less than about 75 nM, preferably less than about 50 nM, preferably less than about 25 nM, preferably less than about 20 nM, preferably less than about 15 nM, preferably less than about 10 nM, preferably less than about 5 nM.

For some applications, preferably the agent has at least about a 25, 50, 75, 100 fold selectivity to the desired target, preferably at least about a 150 fold selectivity to the desired target, preferably at least about a 200 fold selectivity to the desired target, preferably at least about a 250 fold selectivity to the desired target, preferably at least about a 300 fold selectivity to the desired target, preferably at least about a 350 fold selectivity to the desired target.

Female Genitalia

The term “female genitalia” is used in accordance with the definition provided in Gray's Anatomy, C. D. Clemente, 13th American Edition—viz:
“The genital organs consist of an internal and external group. The internal organs are situated within the pelvis and consist of ovaries, the uterine tubes, uterus and the vagina. The external organs are superficial to the urogenital diaphragm and below the pelvic arch. They comprise the mons pubis, the labia majora and minora pudendi, the clitoris, the vestibule, the bulb of the vestibule, and the greater vestibular glands”.
   Corpus Cavernosum

As used herein, the term “corpus cavernosum” refers inter alia to a mass of tissue found in the penis. In this regard, the body of the penis is composed of three cylindrical masses of tissue, each surrounded by fibrous tissue called the tunica albuginea. The paired dorsolateral masses are called the corpora cavernosa penis (corpora=main bodies; cavernosa=hollow); the smaller midventral mass, the corpus spongiosum penis contains the spongy urethra and functions in keeping the spongy urethra open during ejaculation. All three masses are enclosed by fascia and skin and consist of erectile tissue permeated by blood sinuses. The corpus cavernosum comprises smooth muscle cells. The term “corpus cavernosum” as used herein also includes the equivalent smooth muscle cells and/or tissue in the clitoris.

Erectile Dysfunction (ED)

As used herein, the term “erectile dysfunction (ED)” includes both penile erectile dysfunction—characterised by the consistent inability of an adult male to ejaculate or to attain or hold an erection long enough for sexual intercourse—and clitoral dysfunction in the female in so far as there is substantial equivalence between penile and clitoral erectile tissue.

Penile Erection

As used herein, the term “penile erection” refers to the situation whereby, upon stimulation, which may be visual, tactile, auditory, olfactory or from the imagination, the arteries supplying the penis dilate and large quantities of blood enter the blood sinuses. Expansion of these spaces compresses the veins draining the penis, so blood outflow is slowed. These vascular changes, due to a parasympathetic reflex, result in an erection. The penis returns to its flaccid state when the arteries constrict and pressure on the veins is relieved. As used herein, the term “penile” and “penile erection” may be interpreted to apply equally to clitoris in so far as there is substantial equivalence between penile and clitoral erectile tissue.

Clitoris

As used herein, the term “clitoris” refers to the female mass of erectile tissue which is homologous to the penis in the male. Like the male structure, the clitoris is capable of enlargement upon tactile stimulation and plays a role in sexual excitement in the female. In certain types of female sexual dysfunction (FSD), such as female sexual arousal dysfunction (FSAD), the arousal dysfunction may be related to a insufficiency in genital blood flow and relaxation of clitoral corpus cavernosum.

Sexual Genitalia

As used herein, the term “sexual genitalia” refers to male and female genitalia such as the penis and clitoris.

Smooth Muscle

As used herein, the term “smooth muscle” refers to a tissue specialised for contraction composed of smooth muscle fibres (cells) which are located in the walls of hollow internal organs and innervated by autonomic motor neurons. The term “smooth muscle” means muscle lacking striations, hence giving it a smooth appearance. It is also called involuntary muscle. An increase in the concentration of Ca²⁺ in smooth muscle cytosol initiates contraction, just as in striated muscle. However, sacroplasmic reticulum (the reservoir for Ca²⁺ in striated muscle) is scanty in smooth muscle. Calcium ions flow into smooth muscle cytosol from both the extracellular fluid and sarcoplasmic reticulum, but because there are no transverse tubules in smooth muscle fibres, it takes longer for Ca²⁺ to reach the filaments in the centre of the fibre and trigger the contractile process. This accounts, in part, for the slow onset and prolonged contraction of smooth muscle.

Contraction and Relaxation

Several mechanisms regulate contraction and relaxation of smooth muscle cells. In one, a regulatory protein called calmodulin binds to Ca²⁺ in the cytosol. Not only do calcium ions enter smooth muscle fibres slowly, but they also move slowly out of the muscle fibre when excitation declines, which delays relaxation. The prolonged presence of Ca²⁺ in the cytosol provides for smooth muscle tone, a state of continued partial contraction. Smooth muscle tissue is located in the walls of hollow internal organs such as blood vessels, airways to the lungs, the stomach, intestinal gall bladder, urinary bladder, the corpus cavernosa of the penis and the clitoris.

Treatment

It is to be appreciated that all references herein to treatment include one or more of curative, palliative and prophylactic treatment.

Sexual Stimulation

The present invention also encompasses use as defined hereinbefore via administration of a selective D3 dopamine receptor agonist (and a PDEi, preferably a PDE5i or other auxiliary agent where applicable) before and/or during sexual stimulation. Here the term “sexual stimulation” may be synonymous with the term “sexual arousal”. This aspect of the present invention is advantageous because it provides systemic (physiological) selectivity. The natural cascade only occurs at the genitalia and not in other locations—e.g. in the heart etc. Hence, it is possible to achieve a selective effect on the genitalia via the sexual dysfunction (particularly MED or FSAD and/or HSDD) treatment according to the present invention.

Thus, according to the present invention it is highly desirable that there is a sexual stimulation step at some stage. We have found that this step can provide systemic selectivity. Here, “sexual stimulation” may be one or more of a visual stimulation, a physical stimulation, an auditory stimulation, or a thought stimulation.

Agent

Agents for use in the treatment of male sexual dysfunction, in particular MED, or female sexual dysfunction, in particular FSAD and/or HSDD, according to the present invention may be any suitable agent that can act as a selective dopamine D3 receptor agonist and, where appropriate, a combination of a selective dopamine D3 receptor agonist and an auxiliary agent, such as PDEi, preferably a PDE5i. As used herein, the term “agent” includes any entity capable of selectively activating or initiating a dopamine D3 receptor.

Such agents (i.e. the agents as defined above) can be an amino acid sequence or a chemical derivative thereof. The substance may even be an organic compound or other chemical. The agent may even be a nucleotide sequence—which may be a sense sequence or an anti-sense sequence. The agent may even be an antibody.

Thus, the term “agent” includes, but is not limited to, a compound which may be obtainable from or produced by any suitable source, whether natural or not.

The agent may be designed or obtained from a library of compounds which may comprise peptides, as well as other compounds, such as small organic molecules, such as lead compounds.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, or a peptide synthesised synthetically (such as, by way of example, either using a peptide synthesiser or by recombinant techniques or combinations thereof, a recombinant agent, an antibody, a natural or a non-natural agent, a fusion protein or equivalent thereof and mutants, derivatives or combinations thereof.

As used herein, the term “agent” may be a single entity or it may be a combination of agents.

If the agent is an organic compound then for some applications—such as if the agent is a specific dopamine D3 receptor agonist—that organic compound may typically comprise two or more linked hydrocarbyl groups. For some applications, preferably the agent comprises at least two cyclic groups—optionally wherein one of which cyclic groups may be a fused cyclic ring structure. For some applications, at least one of the cyclic groups is a heterocyclic group. For some applications, the heterocyclic group may comprise at least one N in the ring. Examples of such compounds are presented herein.

If the agent is an organic compound then for some applications—such as if the agent is a PDE5i—that organic compound may typically comprise two or more linked hydrocarbyl groups. For some applications, preferably the agent comprises at least two cyclic groups—wherein one of which cyclic groups may be a fused cyclic ring structure. For some applications, preferably at least one of the cyclic groups is a heterocyclic group. For some applications, preferably the heterocyclic group comprises at least one N in the ring. Examples of such compounds are presented in the PDE5 section herein.

The agent may contain halo groups. Here, “halo” means fluoro, chloro, bromo or iodo.

The agent may contain one or more of alkyl, alkoxy, alkenyl, alkylene and alkenylene groups—which may be unbranched- or branched-chain.

Substituted

For the avoidance of doubt, unless otherwise indicated, the term substituted means substituted by one or more defined groups. In the case where groups may be selected from a number of alternative groups, the selected groups may be the same or different. For the avoidance of doubt, the term independently means that where more than one substituent is selected from a number of possible substituents, those substituents may be the same or different.

Pharmaceutically Acceptable Salt

The agent may be in the form of—and/or may be administered as—a pharmaceutically acceptable salt—such as an acid addition salt or a base salt—or a solvate thereof, including a hydrate thereof. For a review on suitable salts see Berge et al, J. Pharm. Sci., 1977, 66, 1-19.

Typically, a pharmaceutically acceptable salt may be readily prepared by using a desired acid or base, as appropriate. The salt may precipitate from solution and be collected by filtration or may be recovered by evaporation of the solvent.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases which form non-toxic salts and examples are the sodium, potassium, aluminium, calcium, magnesium, zinc, diolamine, olamine, ethylenediamine, tromethamine, chloine, megulamine and diethanolamine salts. For reviews on suitable pharmaceutical salts see Berge et al J. Pharm. Sci., 66, 1-19 (1977); Gould P. L., International J. of Pharmaceutics, 33 (1986), 201-217; and Bighley et al., Encyclopedia of Pharmaceutical Technology, Marcel Dekker Inc., New York (1996), Vol. 13, page 453-497. A preferred salt is the sodium salt.

The pharmaceutically acceptable solvates of the compound of the invention include the hydrates thereof.

Hereinafter, compounds, their pharmaceutically acceptable salts, their solvates and polymorphs, defined in any aspect of the invention (except intermediate compounds in chemical processes) are referred to as “compounds of the invention”.

Polymorphic Form(s)/Asymmetric Carbon(s)

The agent may exist in polymorphic form.

The agent may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

Isotopic Variations

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent and pharmaceutically acceptable salts thereof can generally be prepared by conventional procedures using appropriate isotopic variations of suitable reagents.

Prodrugs

It will be appreciated by those skilled in the art that the agent may be derived from a prodrug. Examples of prodrugs include entities that have certain protected group(s) and which may not possess pharmacological activity as such, but may, in certain instances, be administered (such as orally or parenterally) and thereafter metabolised in the body to form the agent which are pharmacologically active.

All protected derivatives and prodrugs of compounds of the present invention are included within the scope of the invention.

Pro-Moieties

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the agents. Such prodrugs are also included within the scope of the invention.

Inhibitor/Antagonist

The term inhibitor as used herein, for example with regard to PDEi or PDE5i compounds and other auxiliary active agents is to be regarded as being interchangeable with the term antagonist.

As used herein, the term “antagonist” means any agent that reduces the action of another agent or target. The antagonistic action may result form a combination of the substance being antagonised (chemical antagonism) or the production of an opposite effect through a different target (functional antagonism or physiological antagonism) or as a consequence of competition for the binding site of an intermediate that links target activation to the effect observed (indirect antagonism).

Further the phrase, enhancing the endogenous erectile process, is to be regarded as being interchangeable with the phrase upregulation of the endogenous erectile process.

Agonist

As used herein the term “agonist” means any agent that enhances the action of or activates another agent or target. The term agonist includes a ligand that binds to receptors and thereby alters, typically increases, the proportion of them that are in an active form, resulting in a biological response.

Pharmaceutical Compositions

The present invention also provides a pharmaceutical composition comprising a therapeutically effective amount of the agent of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

In a preferred embodiment, the agents of the present invention are delivered systemically (such as orally, buccally, sublingually), more preferably orally.

Hence, preferably the agent is in a form that is suitable for oral delivery.

Administration

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The agents of the present invention may be administered alone but will generally be administered as a pharmaceutical composition—e.g. when the agent is in admixture with a suitable pharmaceutical excipient, diluent or carrier selected with regard to the intended route of administration and standard pharmaceutical practice.

For example, the agent can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, penile, vaginal, epidural, sublingual.

It is to be understood that not all of the agents need be administered by the same route. Likewise, if the composition comprises more than one active component, then those components may be administered by different routes.

If the agent of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent; and/or by using infusion techniques.

For parenteral administration, the agent is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the agent of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the agent of the present invention can be administered in the form of a suppository or pessary, or it may be applied topically in the form of a gel, hydrogel, lotion, solution, cream, ointment or dusting powder. The agent of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compositions of the present invention may be administered by direct injection.

For some applications, preferably the agent is administered orally.

For some applications, preferably the agent is administered topically.

Dose Levels

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The agent and/or the pharmaceutical composition of the present invention may be administered in accordance with a regimen of from 1 to 10 times per day, such as once or twice per day.

For oral and parenteral administration to humans, the daily dosage level of the agent may be in single or divided doses.

Depending upon the need, the agent may be administered at a dose of from 0.01 to 30 mg/kg body weight, such as from 0.1 to 10 mg/kg, more preferably from 0.1 to 1 mg/kg body weight. Naturally, the dosages mentioned herein are exemplary of the average case. There can, of course, be individual instances where higher or lower dosage ranges are merited.

Preferably, depending upon the need, the agent may be administered at a dose of from 0.01 to 10 mg/dose, such as from 0.1 to 5 mg/dose, more preferably from 1 to 3 mg/dose. Naturally, the dosages mentioned herein are exemplary of the average case. There can, of course, be individual instances where higher or lower dosage ranges are merited. For guidance, pramipexole is dosed at about 0.125-0.25 mg/dose and apomorphine is dosed at about 2-3 mg/dose.

The daily oral dose may be, for instance, between 20-1000 mg, preferably 50-300 mg, for example.

Suitable doses will include those which allow a satisfactory therapeutic ratio between the treatment of male sexual dysfunction, particularly MED, or female sexual dysfunction, particularly FSAD and/or HSDD, and the induction of emesis or other side effects.

Formulation

The agents of the present invention may be formulated into a pharmaceutical composition, such as by mixing with one or more of a suitable carrier, diluent or excipient, by using techniques that are known in the art.

The following present some non-limiting examples of formulations.

Formulation 1: A tablet is prepared using the following ingredients:

| | weight/mg |
|---|---|
| Agent | 250 |
| Cellulose, microcrystalline | 400 |
| Silicon dioxide, fumed | 10 |
| Stearic acid | 5 |
| Total | 665 |

the components are blended and compressed to form tablets each weighing 665 mg.

Formulation 2: An intravenous formulation may be prepared as follows:

| | |
|---|---|
| Agent | 100 mg |
| Isotonic saline | 1,000 ml |

Individual

As used herein, the term “individual” refers to vertebrates, particularly members of the mammalian species. The term includes but is not limited to domestic animals, sports animals, primates and humans.

Bioavailability

Preferably, the compounds of the invention (and combinations) are orally bioavailable. Oral bioavailablity refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailablity.

Dissolution, the solubilisation of the drug by the aqueous contents of the gastro-intestinal tract (GIT), can be predicted from in vitro solubility experiments conducted at appropriate pH to mimic the GIT. Preferably the compounds of the invention have a minimum solubility of 50 mcg/ml. Solubility can be determined by standard procedures known in the art such as described in Adv. Drug Deliv. Rev. 23, 3-25, 1997.

Membrane permeability refers to the passage of the compound through the cells of the GIT. Lipophilicity is a key property in predicting this and is defined by in vitro Log D_{7.4} measurements using organic solvents and buffer. Preferably the compounds of the invention have a Log D_{7.4} of −2 to +4, more preferably −1 to +2. The log D can be determined by standard procedures known in the art such as described in J. Pharm. Pharmacol. 1990, 42:144.

Cell monolayer assays such as CaCO₂ add substantially to prediction of favourable membrane permeability in the presence of efflux transporters such as p-glycoprotein, so-called caco-2 flux. Preferably, compounds of the invention have a caco-2 flux of greater than 2×10⁻⁶ cms⁻¹, more preferably greater than 5×10⁻⁶ cms⁻¹. The caco flux value can be determined by standard procedures known in the art such as described in J. Pharm. Sci, 1990, 79, 595-600

Metabolic stability addresses the ability of the GIT or the liver to metabolise compounds during the absorption process: the first pass effect. Assay systems such as microsomes, hepatocytes etc are predictive of metabolic liability. Preferably the compounds of the Examples show metabolic stability in the assay system that is commensurate with a hepatic extraction of less then 0.5. Examples of assay systems and data manipulation are described in Curr. Opin. Drug Disc. Devel., 201, 4, 36-44, Drug Met. Disp., 2000, 28, 1518-1523.

Because of the interplay of the above processes further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp., 2001, 29, 82-87; J. Med Chem, 1997, 40, 827-829, Drug Met. Disp., 1999, 27, 221-226.

Chemical Synthesis Methods

Typically the selective D3 dopamine receptor agonist (and/or an auxiliary agent, such as PDEi/PDE5i, where applicable) suitable for the use according to the present invention will be prepared by chemical synthesis techniques.

The agent or target or variants, homologues, derivatives, fragments or mimetics thereof may be produced using chemical methods to synthesise the agent in whole or in part. For example, peptides can be synthesised by solid phase techniques, cleaved from the resin, and purified by preparative high performance liquid chromatography (e.g., Creighton (1983) Proteins Structures And Molecular Principles, WH Freeman and Co, New York N.Y.). The composition of the synthetic peptides may be confirmed by amino acid analysis or sequencing (e.g., the Edman degradation procedure; Creighton, supra).

Direct synthesis of the agent or variants, homologues, derivatives, fragments or mimetics thereof can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the agent or any part thereof, may be altered during direct synthesis and/or combined using chemical methods with a sequence from other subunits, or any part thereof, to produce a variant agent or target, such as, for example, a selective dopamine D3 receptor agonist.

In an alternative embodiment of the invention, the coding sequence of the agent target or variants, homologues, derivatives, fragments or mimetics thereof may be synthesised, in whole or in part, using chemical methods well known in the art (see Caruthers M H et al (1980) Nuc Acids Res Symp Ser 215-23, Horn T et al (1980) Nuc Acids Res Symp Ser 225-232).

Mimetic

As used herein, the term “mimetic” relates to any chemical which includes, but is not limited to, a peptide, polypeptide, antibody or other organic chemical which has the same qualitative activity or effect as a reference agent to a target. That is a mimetic may be a functional equivalent to a known agent.

Chemical Derivative

The term “derivative” or “derivatised” as used herein includes chemical modification of an agent. Illustrative of such chemical modifications would be replacement of hydrogen by a halo group, an alkyl group, an acyl group or an amino group.

Chemical Modification

In one embodiment of the present invention, the agent may be a chemically modified agent.

The chemical modification of an agent may either enhance or reduce hydrogen bonding interaction, charge interaction, hydrophobic interaction, Van Der Waals interaction or dipole interaction between the agent and the target.

In one aspect, the identified agent may act as a model (for example, a template) for the development of other compounds.

Targets

In one aspect of the present invention, a D3 dopamine receptor may be used as a target in screens to identify agents capable of activating D3 dopamine receptors. In this regard, the target may comprise an amino acid sequence encoded by the nucleotide sequence shown as SEQ ID NO: 1 or a variant, homologue, derivative or fragment thereof or may comprise an amino acid sequence shown in SEQ ID NO: 2 or a variant, homologue, derivative or fragment thereof, which is prepared by recombinant and/or synthetic means or an expression entity comprising same.

Alternatively, a D3 dopamine receptor may be used to as a target to identify agents capable of mediating an increase in intracavernosal pressure and/or an increase in female genital blood flow leading to vaginal, clitoral and labial engorgement through the activation of dopamine D3 receptors. Also, a D3 dopamine receptor may be used to as a target to identify agents capable of restoring sexual desire through the activation of dopamine D3 receptors. In both of these respects, the target may be suitable tissue extract.

The target may even be a combination of such tissue and/or recombinant targets.

Recombinant Methods

Typically the agent of the present invention may be prepared by recombinant DNA techniques.

In one embodiment, preferably the agent is a dopamine D3 receptor agonist. The dopamine D3 receptor agonist may be prepared by recombinant DNA techniques.

Amino Acid Sequence

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

The amino acid sequence may be prepared isolated from a suitable source, or it may be made synthetically or it may be prepared by use of recombinant DNA techniques.

In one aspect, the present invention provides an amino acid sequence that is capable of acting as a target in an assay for the identification of one or more agents and/or derivatives thereof.

Preferably, the target is a dopamine D3 receptor.

Preferably, the dopamine D3 receptor is an isolated dopamine D3 receptor and/or is purified and/or is non-native.

The dopamine D3 receptor of the present invention may be in a substantially isolated form. It will be understood that the dopamine D3 receptor may be mixed with carriers or diluents which will not interfere with the intended purpose of the receptor and which will still be regarded as substantially isolated. The dopamine D3 receptor of the present invention may also be in a substantially pure form, in which case it will generally comprise the dopamine D3 receptor in a preparation in which more than 90%, e.g. 95%, 98% or 99% of the dopamine D3 receptor in the preparation is a peptide obtainable from the expression of SEQ ID NO: 1 or variants, homologues, derivatives or fragments thereof or a peptide comprising the amino acid sequence shown as SEQ ID NO: 2 or variants, homologues, derivatives or fragments thereof.

Nucleotide Sequence

As used herein, the term “nucleotide sequence” is synonymous with the term “polynucleotide”.

The nucleotide sequence may be DNA or RNA of genomic or synthetic or of recombinant origin. The nucleotide sequence may be double-stranded or single-stranded whether representing the sense or antisense strand or combinations thereof.

For some applications, preferably, the nucleotide sequence is DNA.

For some applications, preferably, the nucleotide sequence is prepared by use of recombinant DNA techniques (e.g. recombinant DNA).

For some applications, preferably, the nucleotide sequence is cDNA.

For some applications, preferably, the nucleotide sequence may be the same as the naturally occurring form for this aspect.

In one aspect, the present invention provides a nucleotide sequence encoding a substance capable of acting as a target in an assay for the identification of one ore more agents and/or derivative thereof.

In one aspect of the present invention the nucleotide sequence encodes a dopamine D3 receptor.

It will be understood by a skilled person that numerous different nucleotide sequences can encode the same target as a result of the degeneracy of the genetic code. In addition, it is to be understood that skilled persons may, using routine techniques, make nucleotide substitutions that do not substantially affect the activity encoded by the nucleotide sequence of the present invention to reflect the codon usage of any particular host organism in which the target is to be expressed. Thus, the terms “variant”, “homologue” or “derivative” in relation to the nucleotide sequence set out in the attached sequence listings include any substitution of, variation of, modification of, replacement of, deletion of or addition of one (or more) nucleic acid from or to the sequence providing the resultant nucleotide sequence encodes a functional target according the present invention (or even an agent according to the present invention if said agent comprises a nucleotide sequence or an amino acid sequence).

As indicated above, with respect to sequence homology, preferably there is at least 75%, more preferably at least 85%, more preferably at least 90% homology to the D3 receptor sequence cross referenced to herein. More preferably there is at least 95%, more preferably at least 98%, homology. Nucleotide homology comparisons may be conducted as described above. A preferred sequence comparison program is the GCG Wisconsin Besffit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length. These sequences could be used a probes, such as in a diagnostic kit.

Variants/Homologues/Derivatives

In addition to the specific nucleotide sequences mentioned herein and amino acid sequences derivable therefrom, the present invention also encompasses the use of variants, homologue and derivatives thereof. Here, the term “homology” can be equated with “identity”.

In the present context, an homologous sequence is taken to include an amino acid sequence which may be at least 75, 85 or 90% identical, preferably at least 95 or 98% identical. In particular, homology should typically be considered with respect to those regions of the sequence known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Besffit program. A new tool, called BLAST 2 Sequences is also available for comparing protein and nucleotide sequence (see FEMS Microbiol Left 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

Hybridisation

The term “hybridisation” as used herein shall include “the process by which a strand of nucleic acid joins with a complementary strand through base pairing” as well as the process of amplification as carried out in polymerase chain reaction (PCR) technologies.

Nucleotide sequences of the invention capable of selectively hybridising to the nucleotide sequences presented herein, or to their complement, will be generally at least 75%, preferably at least 85 or 90% and more preferably at least 95% or 98% homologous to the corresponding complementary nucleotide sequences presented herein over a region of at least 20, preferably at least 25 or 30, for instance at least 40, 60 or 100 or more contiguous nucleotides.

The term “selectively hybridizable” means that the nucleotide sequence, when used as a probe, is used under conditions where a target nucleotide sequence is found to hybridise to the probe at a level significantly above background. The background hybridisation may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridisation conditions are based on the melting temperature (Tm) of the nucleic acid binding complex, as taught in Berger and Kimmel (1987, Guide to Molecular Cloning Techniques, Methods in Enzymology, Vol. 152, Academic Press, San Diego Calif.), and confer a defined “stringency” as explained below.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridisation can be used to identify or detect identical nucleotide sequences while an intermediate (or low) stringency hybridisation can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Nucleotide sequences which are not 100% homologous to the sequences of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the nucleotide sequence set out in herein under conditions of medium to high stringency. Similar considerations apply to obtaining species homologues and allelic variants of the amino acid and/or nucleotide sequences of the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences, such as the nucleotide sequence set out in SEQ ID NO: 1 of the sequence listings of the present invention. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the protein encoded by the nucleotide sequences.

The nucleotide sequences of the present invention may be used to produce a primer, e.g. a PCR primer, a primer for an alternative amplification reaction, a probe e.g. labelled with a revealing label by conventional means using radioactive or non-radioactive labels, or the nucleotide sequences may be cloned into vectors. Such primers, probes and other fragments will be at least 15, preferably at least 20, for example at least 25, 30 or 40 nucleotides in length, and are also encompassed by the term nucleotide sequence of the invention as used herein.

The nucleotide sequences such as a DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Due to the inherent degeneracy of the genetic code, other DNA sequences which encode substantially the same or a functionally equivalent amino acid sequence, may be used to clone and express the target sequences. As will be understood by those of skill in the art, for certain expression systems, it may be advantageous to produce the target sequences with non-naturally occurring codons. Codons preferred by a particular prokaryotic or eukaryotic host (Murray E et al (1989) Nuc Acids Res 17:477-508) can be selected, for example, to increase the rate of the target expression or to produce recombinant RNA transcripts having desirable properties, such as a longer half-life, than transcripts produced from naturally occurring sequence.

Vector

In one embodiment of the present invention, an agent (i.e. a dopamine D3 receptor agonist) may be administered directly to an individual.

In another embodiment of the present invention, a vector comprising a nucleotide sequence encoding an agent of the present invention is administered to an individual.

Preferably the recombinant agent is prepared and/or delivered to a target site using a genetic vector.

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host and/or a target cell for the purpose of replicating the vectors comprising the nucleotide sequences of the present invention and/or expressing the proteins of the invention encoded by the nucleotide sequences of the present invention. Examples of vectors used in recombinant DNA techniques include but are not limited to plasmids, chromosomes, artificial chromosomes or viruses.

The term “vector” includes expression vectors and/or transformation vectors.

The term “expression vector” means a construct capable of in vivo or in vitro/ex vivo expression.

The term “transformation vector” means a construct capable of being transferred from one species to another.

Naked DNA

The vectors comprising nucleotide sequences encoding an agent of the present invention for use in treating male sexual dysfunction, such as MED, or female sexual dysfunction, such as FSAD, may be administered directly as “a naked nucleic acid construct”, preferably further comprising flanking sequences homologous to the host cell genome.

As used herein, the term “naked DNA” refers to a plasmid comprising a nucleotide sequence encoding an agent of the present invention together with a short promoter region to control its production. It is called “naked” DNA because the plasmids are not carried in any delivery vehicle. When such a DNA plasmid enters a host cell, such as a eukaryotic cell, the proteins it encodes (such as an agent of the present invention) are transcribed and translated within the cell.

Non-Viral Delivery

Alternatively, the vectors comprising nucleotide sequences of the present invention or an agent of the present invention (i.e. selective dopamine D3 receptor agonists) or a target of the present invention (i.e. selective dopamine D3 receptor agonists) may be introduced into suitable host cells using a variety of non-viral techniques known in the art, such as transfection, transformation, electroporation and biolistic transformation.

As used herein, the term “transfection” refers to a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), multivalent cations such as spermine, cationic lipids or polylysine, 1,2,-bis (oleoyloxy)-3-(trimethylammonio) propane (DOTAP)-cholesterol complexes (Wolff and Trubetskoy 1998 Nature Biotechnology 16: 421) and combinations thereof.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Viral Vectors

Alternatively, the vectors comprising an agent or target of the present invention or nucleotide sequences of the present invention may be introduced into suitable host cells using a variety of viral techniques which are known in the art, such as for example infection with recombinant viral vectors such as retroviruses, herpes simplex viruses and adenoviruses.

Preferably the vector is a recombinant viral vectors. Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors (see Kestler et al 1999 Human Gene Ther 10(10):1619-32). In the case of viral vectors, delivery of the nucleotide sequence encoding the agent of the present invention is mediated by viral infection of a target cell.

Targeted Vector

The term “targeted vector” refers to a vector whose ability to infect/transfect/transduce a cell or to be expressed in a host and/or target cell is restricted to certain cell types within the host organism, usually cells having a common or similar phenotype.

Replication Vectors

The nucleotide sequences encoding an agent (i.e. a selective dopamine D3 receptor agonist and/or auxiliary agent, such as PDEi or PDE5i) of the present invention or a target (such as a dopamine D3 receptor) may be incorporated into a recombinant replicable vector. The vector may be used to replicate the nucleotide sequence in a compatible host cell. Thus in one embodiment of the present invention, the invention provides a method of making a target of the present invention by introducing a nucleotide sequence of the present invention into a replicable vector, introducing the vector into a compatible host cell, and growing the host cell under conditions which bring about replication of the vector. The vector may be recovered from the host cell.

Expression Vector

Preferably, an agent of the present invention or a nucleotide sequence of present invention or a target of the present invention which is inserted into a vector is operably linked to a control sequence that is capable of providing for the expression of the coding sequence, such as the coding sequence of the D3 dopamine receptor of the present invention by the host cell, i.e. the vector is an expression vector. An agent of the present invention or a target produced by a host recombinant cell may be secreted or may be contained intracellularly depending on the sequence and/or the vector used. As will be understood by those of skill in the art, expression vectors containing an agent or target of the present invention coding sequences can be designed with signal sequences which direct secretion of the agent or target of the present invention coding sequences through a particular prokaryotic or eukaryotic cell membrane.

Expression In Vitro

The vectors of the present invention may be transformed or transfected into a suitable host cell and/or a target cell as described below to provide for expression of an agent or a target of the present invention. This process may comprise culturing a host cell and/or target cell transformed with an expression vector under conditions to provide for expression by the vector of a coding sequence encoding an agent or a target of the present invention and optionally recovering the expressed agent or target of the present invention. The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. The expression of an agent of the present invention or target of the present invention may be constitutive such that they are continually produced, or inducible, requiring a stimulus to initiate expression.

In the case of inducible expression, production of an agent of the present invention or a target can be initiated when required by, for example, addition of an inducer substance to the culture medium, for example dexamethasone or IPTG.

Fusion Proteins

The dopamine D3 receptor or an agent (i.e. a selective dopamine D3 receptor agonist) of the present invention may be expressed as a fusion protein to aid extraction and purification and/or delivery of the agent of the present invention or the dopamine D3 receptor target to an individual and/or to facilitate the development of a screen for agents. Examples of fusion protein partners include glutathione-5-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the activity of the target.

The fusion protein may comprise an antigen or an antigenic determinant fused to the substance of the present invention. In this embodiment, the fusion protein may be a non-naturally occurring fusion protein comprising a substance which may act as an adjuvant in the sense of providing a generalised stimulation of the immune system. The antigen or antigenic determinant may be attached to either the amino or carboxy terminus of the substance.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognised by a commercially available antibody.

Host Cells

A wide variety of host cells can be employed for expression of the nucleotide sequences encoding the agent—such as an agent of the present invention—or a dopamine D3 receptor target of the present invention. These cells may be both prokaryotic and eukaryotic host cells. Suitable host cells include bacteria such as E. coli, yeast, filamentous fungi, insect cells, mammalian cells, typically immortalised, e.g., mouse, CHO, human and monkey cell lines and derivatives thereof.

Examples of suitable expression hosts within the scope of the present invention are fungi such as Aspergillus species (such as those described in EP-A-0184438 and EP-A-0284603) and Trichoderma species; bacteria such as Bacillus species (such as those described in EP-A-0134048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus oryzae, Trichoderma reesei, Bacillus subtilis, Bacillus licheniformis, Bacillus amyloliquefaciens, Kluyveromyces lactis and Saccharomyces cerevisiae.

The use of suitable host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

Preferred host cells are able to process the expression products to produce an appropriate mature polypeptide. Examples of processing includes but is not limited to glycosylation, ubiquitination, disulfide bond formation and general post-translational modification.

Antibodies

In one embodiment of the present invention, the agent may be an antibody. In addition, or in the alternative, the target may be an antibody.

Antibodies may be produced by standard techniques, such as by immunisation with the substance of the invention or by using a phage display library.

For the purposes of this invention, the term “antibody”, unless specified to the contrary, includes but is not limited to, polyclonal, monoclonal, chimeric, single chain, Fab fragments, fragments produced by a Fab expression library, as well as mimetics thereof. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies. Neutralising antibodies, i.e., those which inhibit biological activity of the substance polypeptides, are especially preferred for diagnostics and therapeutics.

If polyclonal antibodies are desired, a selected mammal (e.g., mouse, rabbit, goat, horse, etc.) is immunised with an immunogenic polypeptide bearing a epitope(s) obtainable from an identified agent and/or substance of the present invention. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. If serum containing polyclonal antibodies to an epitope obtainable from an identified agent and/or substance of the present invention contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against epitopes obtainable from an identified agent and/or substance of the present invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies to the substance and/or identified agent may be prepared using any technique which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies, both monoclonal and polyclonal, which are directed against epitopes obtainable from an identified agent and/or substance are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for the substance may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse W D et al (1989) Science 256:1275-128 1).

Reporters

A wide variety of reporters may be used in the assay methods (as well as screens) of the present invention with preferred reporters providing conveniently detectable signals (e.g. by spectroscopy). By way of example, a reporter gene may encode an enzyme which catalyses a reaction which alters light absorption properties.

Examples of reporter molecules include but are not limited to β-galactosidase, invertase, green fluorescent protein, luciferase, chloramphenicol, acetyltransferase, β-glucuronidase, exo-glucanase and glucoamylase.

Alternatively, radiolabelled or fluorescent tag-labelled nucleotides can be incorporated into nascent transcripts which are then identified when bound to oligonucleotide probes.

In one preferred embodiment, the production of the reporter molecule is measured by the enzymatic activity of the reporter gene product, such as α-galactosidase.

A variety of protocols for detecting and measuring the expression of the target, such as by using either polyclonal or monoclonal antibodies specific for the protein, are known in the art. Examples include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes on polypeptides is preferred, but a competitive binding assay may be employed. These and other assays are described, among other places, in Hampton R et al (1990, Serological Methods, A Laboratory Manual, APS Press, St Paul Minn.) and Maddox D E et al (1983, J Exp Med 15 8:121 1).

A wide variety of labels and conjugation techniques are known by those skilled in the art and can be used in various nucleic and amino acid assays. Means for producing labelled hybridisation or PCR probes for detecting the target polynucleotide sequences include oligolabelling, nick translation, end-labelling or PCR amplification using a labelled nucleotide. Alternatively, the coding sequence, or any portion of it, may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides.

A number of companies such as Pharmacia Biotech (Piscataway, N.J.), Promega (Madison, Wis.), and US Biochemical Corp (Cleveland, Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241. Also, recombinant immunoglobulins may be produced as shown in U.S. Pat. No. 4,816,567.

Additional methods to quantify the expression of a particular molecule include radiolabeling (Melby P C et a/1993 J Immunol Methods 159:235-44) or biotinylating (Duplaa C et al 1993 Anal Biochem 229-36) nucleotides, coamplification of a control nucleic acid, and standard curves onto which the experimental results are interpolated. Quantification of multiple samples may be speeded up by running the assay in an ELISA format where the oligomer of interest is presented in various dilutions and a spectrophotometric or calorimetric response gives rapid quantification.

Although the presence/absence of marker gene expression suggests that the gene of interest is also present, its presence and expression should be confirmed. For example, if the nucleotide sequence is inserted within a marker gene sequence, recombinant cells containing the same may be identified by the absence of marker gene function. Alternatively, a marker gene can be placed in tandem with a target coding sequence under the control of a single promoter. Expression of the marker gene in response to induction or selection usually indicates expression of the target as well.

Alternatively, host cells which contain the coding sequence for the target and express the target coding regions may be identified by a variety of procedures known to those of skill in the art. These procedures include, but are not limited to, DNA-DNA or DNA-RNA hybridisation and protein bioassay or immunoassay techniques which include membrane-based, solution-based, or chip-based technologies for the detection and/or quantification of the nucleic acid or protein.

Screens

Any one or more of appropriate targets—such as an amino acid sequence of and/or a nucleotide sequence encoding a dopamine D3 receptor and/or dopamine D2 receptor—may be used for identifying an agent, e.g. a selective dopamine D3 receptor agonist, in any of a variety of drug screening techniques. The target employed in such a test may be free in solution, affixed to a solid support, borne on a cell surface, or located intracellularly. The target may even be within an animal model, wherein said target may be an exogenous target or an introduced target. The animal model will be a non-human animal model. The abolition of target activity or the formation of binding complexes between the target and the agent being tested may be measured.

Techniques for drug screening may be based on the method described in Geysen, European Patent Application 84/03564, published on Sep. 13, 1984. In summary, large numbers of different small peptide test compounds are synthesised on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques. Alternatively, non-neutralising antibodies can be used to capture the peptide and immobilise it on a solid support.

This invention also contemplates the use of competitive drug screening assays in which neutralising antibodies capable of binding a target specifically compete with a test compound for binding to a target.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

In a preferred aspect, the screen of the present invention comprises at least the following steps (which need not be in this same consecutive order): (a) conducting an in vitro screen to determine whether a candidate agent has the relevant activity (such as modulation of the activity of dopamine D3 receptors); (b) conducting one or more selectivity screens to determine the selectivity of said candidate agent (e.g. to see if said agent is also an agonist in respect of dopamine D2 receptors—such as by using the functional assay protocol presented herein); and (c) conducting an in vivo screen with said candidate agent (e.g. using a functional animal model). Typically, if said candidate agent passes screen (a) and screen (b) then screen (c) is performed.

Diagnostics

The present invention also provides a diagnostic composition or kit for the detection of a pre-disposition for sexual dysfunction, in particular MED or FSAD and/or HSDD. In this respect, the composition or kit will comprise an entity that is capable of indicating the presence, or even the absence, of nitric oxide (NO) and/or one or more vasoactive intestinal proteins (VIPs) in a test sample. Preferably, the test sample is obtained from the penis or the female genitalia. Preferably, the test sample is obtained during sexual arousal.

In order to provide a basis for the diagnosis of disease, normal or standard values from a subject not suffering from sexual dysfunction, in particular either MED or FSAD and/or HSDD, should be established. For example, this may be accomplished by combining body fluids or cell extracts taken from normal subjects, either animal or human, with an antibody to a VIP, for example, under conditions suitable for complex formation which are well known in the art. The amount of standard complex formation may be quantified by comparing it to a dilution series of positive controls where a known amount of antibody is combined with known concentrations of a purified VIP. Then, standard values obtained from normal samples may be compared with values obtained from samples from subjects potentially affected by male sexual dysfunction, such as MED, or female sexual dysfunction, such as FSAD and/or HSDD. Deviation between standard and subject values establishes the presence of the disease state.

The diagnostics may be tailored to evaluate the efficacy of a particular therapeutic treatment regime (i.e. the administration of a selective dopamine D3 agonist) and may be used in animal studies, in clinical trials, or in monitoring the treatment of an individual. If MED or FSAD and/or HSDD is established, a therapeutic agent, for example a selective dopamine D3 agonist according to the present invention, may be administered, and treatment profile or values may be generated. Finally, the diagnostic assay may be repeated on a regular basis to evaluate whether the values progress toward or return to the normal or standard patterns. Successive treatment profiles may be used to show the efficacy of treatment over a period of several days or several months.

Diagnostic Kits

The present invention also includes a diagnostic composition or diagnostic methods or kits for (i) detection and measurement of dopamine D3 receptor activity in biological fluids and tissue; and/or (ii) the detection of a predisposition to a male sexual dysfunction, such as MED, or a female sexual dysfunction, such as FSAD and/or HSDD. In this respect, the composition or kit will comprise an entity that is capable of indicating the presence, or absence, of NO and/or one or more VIPs in a test sample. Preferably, the test sample is obtained from male or female sexual genitalia or a secretion thereof or therefrom. Preferably the test sample is obtained during sexual arousal of the subject.

Diagnostic Testing

In order to provide a basis for the diagnosis of disease, normal or standard values of NO and/or one or more VIPs for one or more subjects not suffering from sexual dysfunction, in particular either MED or FSAD and/or HSDD, should be established. For example, this may be accomplished by combining body fluids or cell extracts taken from normal subjects, either animal or human, with an antibody to a VIP, for example, under conditions suitable for complex formation which are well known in the art. The amount of standard complex formation may be quantified by comparing it to a dilution series of positive controls where a known amount of antibody is combined with known concentrations of a purified VIP. Then, standard values obtained from normal samples may be compared with values obtained from samples from subjects potentially affected by male sexual dysfunction, such as MED, or female sexual dysfunction, such as FSAD and/or HSDD. Deviation between standard and subject values establishes the presence of the disease state.

The diagnostic compositions and/or kits comprising these entities may be used for a rapid, reliable, sensitive, and specific measurement and localisation of NO and/or one or more VIPs in erectile tissue extracts. In certain situations, the kit may indicate the existence of sexual dysfunction, such as MED or FSAD and/or HSDD.

Assay Methods

The diagnostic compositions and/or methods and/or kits may be used in the following techniques which include but are not limited to; competitive and non-competitive assays, radioimmunoassay, bioluminescence and chemiluminescence assays, fluorometric assays, sandwich assays, immunoradiometric assays, dot blots, enzyme linked assays including ELISA, microliter plates, antibody coated strips or dipsticks for rapid monitoring of urine or blood, immunohistochemistry and immunocytochemistry.

Probes

Another aspect of the subject invention is the provision of nucleic acid hybridisation or PCR probes which are capable of detecting (especially those that are capable of selectively selecting) polynucleotide sequences, including genomic sequences, encoding a target coding region, such as a dopamine D3 receptor, or closely related molecules, such as alleles. The specificity of the probe, i.e., whether it is derived from a highly conserved, conserved or non-conserved region or domain, and the stringency of the hybridisation or amplification (high, intermediate or low) will determine whether the probe identifies only naturally occurring target coding sequence, or related sequences. Probes for the detection of related nucleic acid sequences are selected from conserved or highly conserved nucleotide regions of target family members and such probes may be used in a pool of degenerate probes. For the detection of identical nucleic acid sequences, or where maximum specificity is desired, nucleic acid probes are selected from the non-conserved nucleotide regions or unique regions of the target polynucleotides. As used herein, the term “non-conserved nucleotide region” refers to a nucleotide region that is unique to a target coding sequence disclosed herein and does not occur in related family members.

PCR as described in U.S. Pat. No. 4,683,195, U.S. Pat. No. 4,800,195 and U.S. Pat. No. 4,965,188 provides additional uses for oligonucleotides based upon target sequences. Such oligomers are generally chemically synthesised, but they may be generated enzymatically or produced from a recombinant source. Oligomers generally comprise two nucleotide sequences, one with sense orientation (5′->3′) and one with antisense (3′<-5′) employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

The nucleic acid sequence for an agent or a target can also be used to generate hybridisation probes as previously described, for mapping the endogenous genomic sequence. The sequence may be mapped to a particular chromosome or to a specific region of the chromosome using well known techniques. These include in situ hybridisation to chromosomal spreads (Verma et al (1988) Human Chromosomes: A Manual of Basic Techniques, Pergamon Press, New York City), flow-sorted chromosomal preparations, or artificial chromosome constructions such as YACs, bacterial artificial chromosomes (BACs), bacterial PI constructions or single chromosome cDNA libraries.

In situ hybridisation of chromosomal preparations and physical mapping techniques such as linkage analysis using established chromosomal markers are invaluable in extending genetic maps. Examples of genetic maps can be found in Science (1995; 270:410f and 1994; 265:1981f). Often the placement of a gene on the chromosome of another mammalian species may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once a disease or syndrome has been crudely localised by genetic linkage to a particular genomic region any sequences mapping to that area may represent associated or regulatory genes for further investigation. The nucleotide sequence of the subject invention may also be used to detect differences in the chromosomal location due to translocation, inversion, etc. between normal, carrier or affected individuals.

Organism

The term “organism” in relation to the present invention includes any organism that could comprise the target and/or products obtained therefrom. Examples of organisms may include a mammal, a fungus, yeast or a plant.

The term “transgenic organism” in relation to the present invention includes any organism that comprises the target and/or products obtained therefrom.

Transformation of Host Cells/Host Organisms

As indicated earlier, the host organism can be a prokaryotic or a eukaryotic organism. Examples of suitable prokaryotic hosts include E. coli and Bacillus subtilis. Teachings on the transformation of prokaryotic hosts is well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc.

If a prokaryotic host is used then the nucleotide sequence may need to be suitably modified before transformation—such as by removal of introns.

In another embodiment the transgenic organism can be a yeast. In this regard, yeast have also been widely used as a vehicle for heterologous gene expression. The species Saccharomyces cerevisiae has a long history of industrial use, including its use for heterologous gene expression. Expression of heterologous genes in Saccharomyces cerevisiae has been reviewed by Goodey et al (1987, Yeast Biotechnology, D R Berry et al, eds, pp 401-429, Allen and Unwin, London) and by King et al (1989, Molecular and Cell Biology of Yeasts, E F Walton and G T Yarronton, eds, pp 107-133, Blackie, Glasgow).

For several reasons Saccharomyces cerevisiae is well suited for heterologous gene expression. First, it is non-pathogenic to humans and it is incapable of producing certain endotoxins. Second, it has a long history of safe use following centuries of commercial exploitation for various purposes. This has led to wide public acceptability. Third, the extensive commercial use and research devoted to the organism has resulted in a wealth of knowledge about the genetics and physiology as well as large-scale fermentation characteristics of Saccharomyces cerevisiae.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol. 5, Anthony H Rose and J Stuart Harrison, eds, 2nd edition, Academic Press Ltd.).

Several types of yeast vectors are available, including integrative vectors, which require recombination with the host genome for their maintenance, and autonomously replicating plasmid vectors.

In order to prepare the transgenic Saccharomyces, expression constructs are prepared by inserting the nucleotide sequence of the present invention into a construct designed for expression in yeast. Several types of constructs used for heterologous expression have been developed. The constructs contain a promoter active in yeast fused to the nucleotide sequence of the present invention, usually a promoter of yeast origin, such as the GALL promoter, is used. Usually a signal sequence of yeast origin, such as the sequence encoding the SUC2 signal peptide, is used. A terminator active in yeast ends the expression system.

For the transformation of yeast several transformation protocols have been developed. For example, a transgenic Saccharomyces according to the present invention can be prepared by following the teachings of Hinnen et al (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells are selected using various selective markers. Among the markers used for transformation are a number of auxotrophic markers such as LEU2, HIS4 and TRP1, and dominant antibiotic resistance markers such as aminoglycoside antibiotic markers, e.g. G418.

Another host organism is a plant. The basic principle in the construction of genetically modified plants is to insert genetic information in the plant genome so as to obtain a stable maintenance of the inserted genetic material. Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27). Further teachings on plant transformation may be found in EP-A-0449375.

Thus, the present invention also provides a method of transforming a host cell with a nucleotide sequence that is to be the target or is to express the target. Host cells transformed with the nucleotide sequence may be cultured under conditions suitable for the expression and recovery of the encoded protein from cell culture. The protein produced by a recombinant cell may be secreted or may be contained intracellularly depending on the sequence and/or the vector used. As will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain which will facilitate purification of soluble proteins (Kroll D J et al (1993) DNA Cell Biol 12:441-53).

PDE5 Inhibitor—Test Methods

PDE action potency values referred to herein are determined by the following assays:

Phosphodiesterase (PDE) Inhibitory Activity

Preferred PDE compounds suitable for use in accordance with the present invention are potent and selective cGMP PDE5 inhibitors. In vitro PDE inhibitory activities against cyclic guanosine 3′,5′-monophosphate (cGMP) and cyclic adenosine 3′,5′-monophosphate (cAMP) phosphodiesterases can be determined by measurement of their IC₅₀ values (the concentration of compound required for 50% inhibition of enzyme activity).

The required PDE enzymes can be isolated from a variety of sources, including human corpus cavernosum, human and rabbit platelets, human cardiac ventricle, human skeletal muscle and bovine retina, essentially by the method of W. J. Thompson and M. M. Appleman (Biochem., 1971, 10, 311). In particular, the cGMP-specific PDE (PDE5) and the cGMP-inhibited CAMP PDE (PDE3) can be obtained from human corpus cavernosum tissue, human platelets or rabbit platelets; the cGMP-stimulated PDE (PDE2) was obtained from human corpus cavernosum; the calcium/calmodulin (Ca/CAM)-dependent PDE (PDE1) from human cardiac ventricle; the cAMP-specific PDE (PDE4) from human skeletal muscle; and the photoreceptor PDE (PDE6) from bovine retina. Phosphodiesterases 7-11 can be generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors was investigated by assaying a fixed amount of enzyme in the presence of varying inhibitor concentrations and low substrate, (cGMP or cAMP in a 3:1 ratio unlabelled to [³H]-labeled at a conc ˜1/3 Kₘ) such that IC₅₀≅Kᵢ. The final assay volume was made up to 1001 μl with assay buffer [20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension.

Functional Activity

This can be assessed in vitro by determining the capacity of a compound of the invention to enhance sodium nitroprusside-induced relaxation of pre-contracted rabbit corpus cavernosum tissue strips, as described by S. A. Ballard et al. (Brit. J. Pharmacol., 1996, 118 (suppl.), abstract 153P).

The invention will now be further described, by way of example, in which reference is made to the following Figures and List of Sequences:

### FIGURES

FIG. 1 shows the comparative effect of compounds having functionally at least 30-fold selectivity for D3 receptor over D2 receptor on erection versus one or more side effects, such as nausea, vomiting, hypotension or syncope.

FIG. 2 shows that a selective D3 agonist has no significant effects, in contrast to a D3-preferring D2/D3 agonist, on hemodynamic parameters in the anaesthetised dog.

### LIST OF SEQUENCES

SEQ ID NO: 1 shows a nucleotide sequence for a human dopamine D3 receptor;

SEQ ID NO: 2 shows an amino acid sequence for a human dopamine D3 receptor;

SEQ ID NOS: 3 and 4 show nucleotide sequences (cDNAs) coding for human soluble secreted endopeptidase (SEP). SEQ ID NO: 4 includes 5′ and 3′ partial vector sequences (highlighted); and

SEQ ID NO: 5 shows an amino acid sequence of a human SEP protein.

### EXAMPLES

Chemistry Examples

The invention is illustrated by the following non-limiting examples in which the following abbreviations and definitions are used:
α_{D} optical rotation at 587 nm.
Arbacel® filter agent
B broad
Boc tert-butoxycarbonyl
CDCl₃ chloroform-d1
CD₃OD methanol-d4
δ chemical shift
D doublet
Dd double doublet
DCM dichloromethane
DMF N,N-dimethylformamide
DMSO dimethylsulfoxide
H hours
HCl hydrogen chloride
LRMS low resolution mass spectrum
M multiplet
m/z mass spectrum peak
Min minutes
Mpt melting point
NaOH sodium hydroxide
NMR nuclear magnetic resonance
Q quartet
S singlet
T triplet
Tf trifluoromethanesulfonyl
TFA trifluoroacetic acid
THF tetrahydrofuran
TLC thin layer chromatography

Melting points were determined using a Perkin Elmer DSC7 at a heating rate of 20° C./minute).

X-ray diffraction data were recorded at room temperature using a Bruker AXS SMART-APEX CCD area-detector diffractometer (Mo Kα radiation). Intensities were integrated from several series of exposures. Each exposure covered 0.30 in ω, with an exposure time of 60 s and the total data set was more than a sphere.

### Example 1

### 2-Amino-1-(3-methoxyphenyl)ethanol

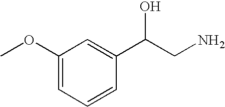

3-Methoxybenzaldehyde (27.2 g, 0.2 mol) in THF (150 ml) was added to a stirred solution of 3N HCl (aq) (150 ml, 0.3 mol) and sodium sulphite (37.8 g, 0.3 mol) at room temperature. After 10 minutes potassium cyanide (19.53 g, 0.3 mol) was added, portion wise, and the reaction mixture was then stirred for 30 minutes. Diethyl ether (800 ml) and water (300 ml) were added and subsequent layers partitioned. Aqueous re-extracted with diethyl ether (500 ml) the organics combined, dried over anhydrous magnesium sulphate, filtered then concentrated in vacuo to give the cyanohydrin intermediate as a colourless oil, (35.57 g, 0.22 mol, >100%). Borane-tetrahydrofuran complex (1M in THF) (400 ml, 0.4 mol) was then cautiously added to the cyanohydrin in THF (100 ml). Once effervescence had ceased, stirring was continued at reflux for 1.5 hours under an atmosphere of nitrogen. The reaction mixture was cooled then quenched with methanol (40 ml) before concentrating in vacuo to give a colourless oil. 6M HCl (aq) (200 ml) was added and reaction stirred at reflux for two hours before concentrating in vacuo to give a white solid. This was pre-absorbed onto silica then purified by column chromatography eluting with dichloromethane: methanol: ammonia (90:10:1) to give the title compound as a colourless oil (31.3 g, 0.19 mol, 94%). ¹H NMR (CDCl₃, 400 MHz) δ: 1.60 (bs, 2H), 2.80 (dd, 1H), 3.02 (dd, 1H), 3.46 (s, 1H), 3.81 (s, 3H), 4.60 (dd, 1H), 6.81 (d, 1H), 6.91 (d, 1H), 6.93 (s, 1H), 7.22 (t, 1H). LRMS: m/z 168 (M−H⁺). Analysis found C, 56.66; H, 8.28; N, 6.91%. C₉H₁₃NO₂.1.33H₂O requires C, 56.33; H, 8.27; N, 7.30%.

### Example 2

### N-[2-Hydroxy-2-(3-methoxyphenyl)ethyl]propionamide

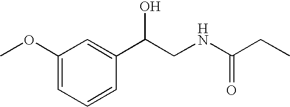

Triethylamine (52 ml, 0.37 mol) was added to the amine from example 1 (31.3 g, 0.19 mol) in dichloromethane (400 ml) and reaction mixture stirred under an atmosphere of nitrogen gas at 0° C. for 10 minutes. Propionyl chloride (16.3 ml, 0.19 mol) was added and after stirring for 30 minutes, the reaction temperature was raised to room temperature for a further 5 hours. The reaction mixture was quenched 1N HCl (aq) (100 ml) and then extracted with dichloromethane (2×50 ml). The organic fractions were combined, dried over anhydrous magnesium sulphate, filtered and concentrated in vacuo to give the title compound as a colourless oil that crystallised on standing to white crystals (28 g, 0.13 mol, 67%). ¹H NMR (CDCl₃, 400 MHz) δ: 1.18 (t, 3H), 2.22 (q, 2H), 2.51 (bs, 1H), 3.31 (m, 1H), 3.71 (dd, 1H), 3.80 (s, 3H), 4.81 (m, 1H), 5.95 (bs, 1H), 6.80 (d, 1H), 6.90 (d, 1H), 6.91 (s, 1H), 7.22 (t, 1H). LRMS: m/z 224. Mpt: 77-78° C. Analysis found C, 63.86; H, 7.82; N, 6.28%. C₁₂H₁₇NO₃0.1H₂O requires C, 64.04; H, 7.70; N, 6.22%.

### Example 3

### 1-(3-Methoxyphenyl)-2-propylaminoethanol

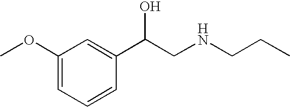

Borane-tetrahydrofuran complex (1M in THF) (376 ml, 0.4 mol) was added to amide from example 2 (28 g, 0.13 mol) in dry THF (100 ml) then the reaction mixture, stirred under an atmosphere of nitrogen gas, was brought to reflux for 2.5 hours. The reaction mixture was cooled then quenched with methanol (40 ml), before concentrating in vacuo to give an opaque white oil. 6N HCl (aq) (200 ml) was added and reaction stirred at reflux for two hours. The reaction mixture was cooled then dichloromethane (200 ml) added and the layers separated. The aqueous layer was rendered basic by addition of potassium carbonate then re-extracted with dichloromethane (2×200 ml). Organic extracts were combined, dried over anhydrous magnesium sulphate, filtered and concentrated in vacuo to give the title compound as a colourless oil that crystallised on standing to give colourless crystals (15.3 g, 0.07 mol, 59%). ¹H NMR (CDCl₃, 400 MHz) δ: 0.93 (t, 3H), 1.62 (q, 2H), 2.71 (q, 2H), 2.81 (t, 2H), 3.00 (d, 1H), 3.80 (s, 3H), 4.30 (bs, 1H), 4.89 (d, 1H), 6.81 (d, 1H), 6.91 (d, 1H), 6.93 (s, 1H), 7.22 (t, 1H). LRMS: m/z 210. Mpt: 50-51° C. Analysis found C, 67.47; H, 9.02; N, 6.45%. C₁₂H₁₉NO₂.0.2H₂O requires C, 67.70; H, 9.19; N, 6.58%.

### Example 4

### 2-Chloro-N-[2-hydroxy-2-(3-methoxyphenyl)ethyl]-N-propylacetamide

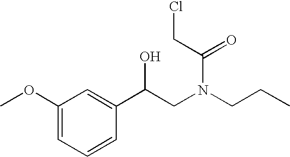

Sodium hydroxide (15.1 g, 0.38 mol) in water (180 ml) was added to the amine from example 3 (15.8 g, 0.08 mol) in dichloromethane (500 ml) and the solution vigorously stirred at room temperature. Chloroacetylchloride (7.22 ml, 0.09 mol) was then added and the reaction mixture stirred for a further 30 minutes. The layers were separated and the aqueous layer re-extracted with dichloromethane (200 ml). The organic extracts were combined, dried over anhydrous magnesium sulphate, filtered and concentrated in vacuo to give the title compound as a colourless oil (17.8 g, 0.06 mol, 83%). ¹H NMR (CDCl₃, 400 MHz) δ: 0.96 (t, 3H), 1.62 (q, 2H), 3.21 (q, 2H), 3.57-3.71 (m, 2H), 3.82 (s, 3H), 4.01-4.21 (bq, 1H), 4.16 (s, 2H), 5.00 (m, 1H), 6.82 (m, 1H), 6.91-6.99 (m, 2H), 7.22 (m, 1H). LRMS: m/z 286. Analysis found C, 57.38; H, 6.95; N, 4.67%. C₁₄H₂₀NO₃Cl.0.33H₂O requires C, 57.64; H, 7.14; N, 4.80%.

### Example 5

### 6-(3-Methoxyphenyl)-4-Propylmorpholin-3-one

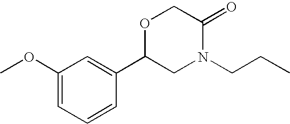

Potassium hydroxide (4.2 g, 0.07 mol), isopropyl alcohol (500 ml) and the amide from example 4 (17.8 g, 0.06 mol) were stirred together as an opaque solution with water (15 ml) for 2 hours. The reaction mixture was concentrated in vacuo and the yellow residue dissolved in ethyl acetate (200 ml). This was partitioned with water (200 ml) then brine (200 ml). The organic fraction was dried over anhydrous magnesium sulphate, filtered and concentrated in vacuo to give the title compound as a yellow oil (15.8 g, 0.06 mol, 100%). ¹H NMR (CDCl₃, 400 MHz) δ: 0.96 (t, 3H), 1.62 (m, 2H), 3.36 (m, 2H), 3.51 (q, 2H), 3.81 (s, 3H), 4.30-4.62 (bq, 2H), 4.79 (d, 1H), 6.85 (d, 1H), 6.91 (d, 1H), 6.95 (s, 1H), 7.29 (t, 1H). LRMS: m/z 272. Analysis found C, 66.80; H, 7.78; N, 5.52%. C₁₄H₁₉NO₃.0.1H₂O requires C, 66.96; H, 7.71; N, 5.58%.

### Example 6

### 2-(3-Methoxyphenyl)-4-propylmorpholine

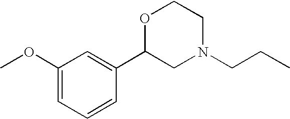

Borane-tetrahydrofuran complex (1M in THF) (200 ml, 0.19 mol) was added dropwise to the morpholin-3-one from example 5 (15.8 g, 0.06 mol) in dry THF (100 ml) under an atmosphere of nitrogen, over 30 minutes. The reaction mixture was brought to reflux for 3 hours then cooled and quenched by addition of methanol (30 ml). The reaction mixture was then concentrated in vacuo and the colourless residue cautiously suspended in 4N HCl (aq) (400 ml) before refluxing for 2.5 hours. The reaction mixture was cooled and dichloromethane (200 ml) added. Layers were separated and the aqueous layer rendered basic by addition of potassium carbonate before re-extracting with dichloromethane (3×100 ml). The organic extracts were combined, dried over anhydrous magnesium sulphate, filtered and concentrated in vacuo to give the title compound as a colourless oil (12.51 g, 0.05 mol, 84%). ¹H NMR (CDCl₃, 400 MHz) δ: 0.95 (t, 3H), 1.59 (q, 2H), 2.05 (t, 1H), 2.23 (t, 1H), 2.40 (t, 2H), 2.81 (d, 1H), 2.98 (d, 1H), 3.80 (s, 3H), 3.85 (t, 1H), 4.05 (d, 1H), 4.60 (d, 1H), 6.81 (d, 1H), 6.91 (d, 1H), 7.21 (t, 1H), 7.23 (s, 1H). LRMS: m/z 236. Analysis found C, 68.94; H, 8.80; N, 5.79%. C₁₄H₂₁NO₂.0.5H₂O requires C, 68.82; H, 9.08; N, 5.73%.

### Example 7a

### R-(−)-3-(4-Propylmorpholin-2-yl)phenol

### Example 7b

### S-(+)-3-(4-Propylmorpholin-2-yl)phenol

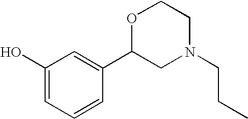

Hydrobromic acid (250 ml) and the anisole from example 6 (8.62 g, 0.03 mol) were heated to reflux together for 1 hour. After cooling the reaction mixture was diluted with water (100 ml) then neutralised by addition of NH₄OH (20 ml). The yellow opaque solution was then extracted with dichloromethane (2×100 ml). The organic extracts were combined then dried over anhydrous magnesium sulphate, filtered and concentrated in vacuo to give the racemic mixture of the title compound as a yellow oil (7.78 g, 0.03 mol, 96%). The enantiomers were separated by chiral chromatography (Chiralpak AD 250* 20 mm column) eluting with hexane: isopropyl alcohol: diethylamine (70:30:0.05) to give enantiomer 1 (ee>99.5%) and enantiomer 2 (ee>99%). Each enantiomer was purified by column chromatography on silica eluting with dichloromethane: methanol (95:5) to give enantiomer 1 (7a) (3.02 g, 0.014 mol, 39%) and enantiomer 2 (7b) (3.15 g, 0.014 mol, 40%) as colourless oils. Enantiomer 1 (7a): ¹H NMR (CDCl₃, 400 MHz) δ: 0.96 (t, 3H), 1.60 (q, 2H), 2.13 (t, 1H), 2.31 (t, 1H), 2.41 (t, 2H), 2.85 (d, 1H), 3.02 (d, 1H), 3.90 (t, 1H), 4.02 (dd, 1H), 4.60 (d, 1H), 6.78 (d, 1H), 6.80 (s, 1H), 6.91 (d, 1H), 7.20 (t, 1H). LRMS: m/z 222 (M−H⁺). Enantiomer 2 (7b): ¹H NMR (CDCl₃, 400 MHz) δ: 0.96 (t, 3H), 1.60 (q, 2H), 2.13 (t, 1H), 2.31 (t, 1H), 2.41 (t, 2H), 2.85 (d, 1H), 3.02 (d, 1H), 3.90 (t, 1H), 4.02 (dd, 1H), 4.60 (d, 1H), 6.78 (d, 1H), 6.80 (s, 1H), 6.91 (d, 1H), 7.20 (t, 1H). LRMS: m/z 222 (M−H⁺).

### Example 8

### R-(−)-3-(4-Propylmorpholin-2-yl)phenol hydrochloride

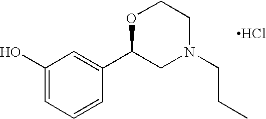

Enantiomer 1 (7a) of example 7 (3.00 g, 0.014 mol) was dissolved in diethyl ether (180 ml) and hydrogen chloride (2.0M solution in diethyl ether) (10 ml) was added. The reaction mixture was stirred at room temperature for 30 minutes, then the solvent was decanted and dried in vacuo, giving title compound as a white solid (3.115 g, 0.012 mol, 90%). ¹H NMR (CD₃OD, 400 MHz) δ: 1.06 (t, 3H), 1.81 (m, 2H), 3.02 (t, 1H), 3.16 (t, 2H), 3.20 (t, 1H), 3.60 (t, 2H), 4.01 (t, 1H), 4.26 (d, 1H), 4.71 (d, 1H), 6.78 (d, 1H), 6.82 (s, 1H), 6.83 (d, 1H), 7.21 (t, 1H). LRMS: m/z 222 (M−H⁺). Analysis found C, 59.74; H, 7.98; N, 5.25%. C₁₃H₁₉NO₂.0.18H₂O requires C, 59.82; H, 7.86; N, 5.37%. α_{D}=−5.66° (Methanol 10.6 mg/10 ml).

A sample of the title compound was re crystallised by vapour diffusion using a methanol: diethyl ether mix and an X-ray crystal structure obtained. The absolute stereochemistry of the title compound was determined from the diffraction data by the method of Flack (H. D. Flack, Acta Cryst. 1983, 439, 876-881) and was shown to have an ‘R’ configuration.

### Example 9

### 2-Amino-1-(3,5-dimethoxyphenyl)ethanol

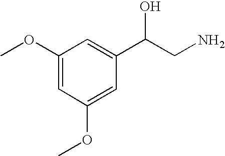

Prepared following the same method as for example 1 starting from 3,5-dimethoxybenzaldehyde (5.00 g, 0.03 mol). After refluxing in 6M HCl (aq) the reaction mixture was cooled and extracted with diethyl ether (2×80 ml). The organic layers were discarded and the aqueous layer basified by the addition of potassium carbonate. The aqueous residue was then extracted with ethyl acetate (3×70 ml). The organic extracts were combined and dried over anhydrous magnesium sulphate, filtered and concentrated in vacuo to give the title compound as a pale yellow oil (3.47 g, 0.018 mol, 59%). ¹H NMR (CD₃OD, 400 MHz) δ: 2.77-2.86 (m, 2H), 3.78 (s, 6H), 4.60 (m, 1H), 6.38 (s, 1H), 6.52 (s, 2H). LRMS: m/z 198 (M−H⁺).

### Example 10

### N-[2-(3,5-dimethoxyphenyl)-2-hydroxyethyl]propionamide

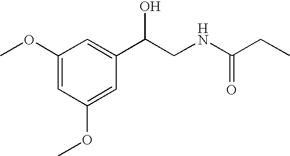

Prepared following the same method as for example 2 starting from the amine in example 9 (3.41 g, 0.017 mol). The crude reaction mixture was purified by column chromatography on silica eluting with dichloromethane: methanol (95:5) to give the title compound as a bright yellow oil (3.08 g, 0.012 mol, 70%). ¹H NMR (CDCl₃, 400 MHz) δ: 1.18 (m, 3H), 2.24 (m, 2H), 3.34 (m, 1H), 3.68 (m, 1H), 3.81 (s, 6H), 4.80 (dd, 1H), 5.95 (bs, 1H), 6.39 (s, 1H), 6.51 (s, 2H). LRMS: m/z 252 (M−H⁻).

### Example 11

### 1-(3,5-dimethoxyphenyl)-2-propylaminoethanol

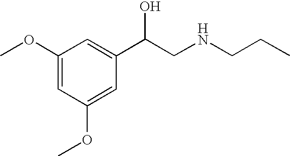

Prepared following the method as for example 3 starting from the amide in example 10 (3.06 g, 0.012 mol) to give the title compound as an orange oil (2.72 g, 0.011 mol, 94%). ¹H NMR (CD₃OD, 400 MHz) δ: 0.95 (t, 3H), 1.56 (m, 2H), 2.61 (m, 2H), 2.77 (d, 2H), 3.78 (s, 6H), 4.70 (t, 1H), 6.38 (s, 1H), 6.51 (s, 2H). LRMS: m/z 240 (M−H⁺).

### Example 12

### 2-Chloro-N-[2-(3,5-dimethoxyphenyl)-2-hydroxyethyl]-N-propylacetamide

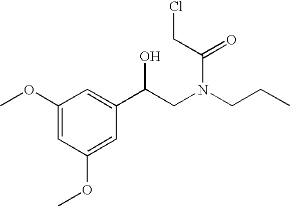

Prepared following the same method as for example 4 starting from the amine in example 11 (2.70 g, 0.011 mol) to give the title compound as a yellow oil (3.56 g, 0.011 mol, 100%). ¹H NMR (CDCl₃, 400 MHz) δ: 0.92 (t, 3H), 1.61 (m, 2H), 3.20 (m, 2H), 3.51-3.64 (m, 2H), 3.80 (d, 6H), 4.13 (s, 2H), 4.95 (m, 1H), 6.40 (m, 1H), 6.55 (s, 2H). LRMS: m/z 316 (M−H⁺).

### Example 13

### 6-(3,5-Dimethoxyphenyl)-4-Propylmorpholin-3-one

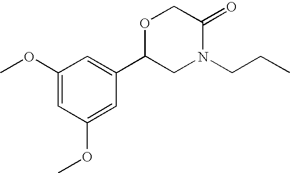

Prepared following the same method as for example 5 starting from the amide in example 12 (3.54 g, 0.011 mol) to give the title compound as a yellow oil (2.44 g, 0.009 mol, 78%). ¹H NMR (CDCl₃, 400 MHz) δ: 0.94 (t, 3H), 1.61 (m, 2H), 3.30 (m, 2H), 3.49 (m, 2H), 3.80 (s, 6H), 4.30 (d, 1H), 4.42 (d, 1H), 4.73 (dd, 1H), 6.42 (s, 1H), 6.53 (s, 2H). LRMS: m/z 280 (M−H⁺).

### Example 14

### 2-(3,5-Dimethoxyphenyl)-4-propylmorpholine

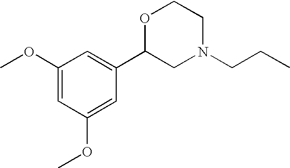

Prepared following the method as for example 6 starting from the amide in example 13 (2.42 g, 0.009 mol). After refluxing in 6M HCl (aq) the cooled reaction mixture was extracted with diethyl ether (2×80 ml). The organic layers were discarded and the aqueous basified by addition of potassium carbonate. The aqueous residue was then extracted with ethyl acetate (3×80 ml) and the organic extracts combined, dried over anhydrous magnesium sulphate, filtered then concentrated in vacuo to give the title compound as a pale orange oil (2.14 g, 0.008 mol, 93%). ¹H NMR (CD₃OD, 400 MHz) δ: 0.95 (t, 3H), 1.58 (m, 2H), 2.01 (m, 1H), 2.22 (dt, 1H), 2.38 (t, 2H), 2.83 (d, 1H), 2.93 (d, 1H), 3.78 (m, 7H), 4.01 (dd, 1H), 4.45 (dd, 1H), 6.39 (s, 1H), 6.49 (s, 2H). LRMS: m/z 266 (M−H⁺).

### Example 15a

### R-5-(4-Propylmorpholin-2-yl)benzene-1,3-diol

### Example 15b

### S-5-(4-Propylmorpholin-2-yl)benzene-1,3-diol

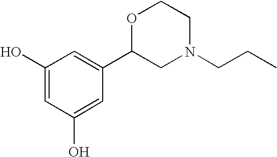

Prepared following the same route as for example 7 starting from the 3,5-dimethoxyphenyl compound in example 14 (1.00 g, 0.004 mol) giving the title racemic compound as a brown oil (145 mg, 0.61 mmol, 16%). The enantiomers were separated by chiral chromatography (Chiralpak AD 250*20 mm column) eluting with hexane: isopropyl alcohol: (80:20) to give enantiomer 1 (15a) (5.2 mg) (ee>98.94%) and enantiomer 2 (15b) (5.1 mg) (ee>96.46%) as brown oils. Enantiomer 1 (15a): ¹H NMR (CD₃OD, 400 MHz) δ: 0.96 (t, 3H), 1.58 (m, 2H), 2.01 (t, 1H), 2.20 (dt, 1H), 2.37 (t, 2H), 2.81-2.92 (m, 2H), 3.89 (dt, 1H), 3.99 (dd, 1H), 4.38 (dd, 1H), 6.18 (t, 1H), 6.26 (s, 2H). LRMS: m/z 238 (M−H⁺). Enantiomer 2 (15b): ¹H NMR (CD₃OD, 400 MHz) δ: 0.95 (t, 3H), 1.58 (m, 2H), 2.01 (t, 1H), 2.20 (dt, 1H), 2.38 (t, 2H), 2.80-2.92 (q, 2H), 3.78 (dt, 1H), 3.98 (dd, 1H), 4.38 (dd, 1H), 6.18 (s, 1H), 6.25 (s, 2H). LRMS: m/z 238 (M−H⁺).

### Example 16

### 4-Fluoro-3-methoxybenzaldehyde

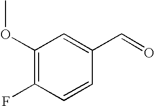

(4-Fluoro-3-methoxyphenyl) methanol (5.00 g, 0.03 mol) and manganese dioxide (33.4 g, 0.38 mol) were stirred in dichloromethane (100 ml) under an atmosphere of nitrogen, at gentle reflux for 16 hours. The cooled reaction mixture was then filtered through arbacel and concentrated in vacuo to give the title compound as a white solid (4.18 g, 0.027 mol, 85%). ¹H NMR (CDCl₃, 400 MHz) δ: 3.96 (s, 3H), 7.23 (d, 1H), 7.43 (m, 1H), 7.50 (d, 1H) 9.91 (s, 1H). Mpt: 61-63° C. Analysis found C, 62.18; H, 4.54%. C₈H₇FO₂ requires C, 62.34; H, 4.58%.

### Example 17

### 2-Amino-1-(4-fluoro-3-methoxyphenyl)ethanol

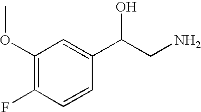

Prepared following the same method as for example 1 starting from 4-fluoro-3-methoxybenzaldehyde (4.17 g, 0.03 mol). After refluxing in 6M HCl (aq) the reaction mixture was cooled and extracted with diethyl ether (2×60 ml). The organic layers were discarded and the aqueous layer basified by the addition of potassium carbonate. The aqueous residue was then extracted with ethyl acetate (3×80 ml). The organic extracts were combined and dried over anhydrous magnesium sulphate, filtered and concentrated in vacuo to give the title compound as an orange oil (2.36 g, 0.013 mol, 47%). ¹H NMR (CD₃OD, 400 MHz) δ: 2.80-2.91 (m, 2H), 3.86 (s, 3H), 4.64 (m, 1H), 6.89 (m, 1H), 7.03 (t, 1H), 7.11 (dd, 1H). LRMS: m/z 186 (M−H⁺).

### Example 18

### N-[2-(4-Fluoro-3-methoxyphenyl]-2-hydroxyethyl]propionamide

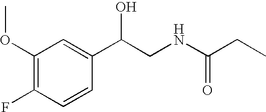

Prepared following the same method as for example 2 starting with the amine from example 17 (1.32 g, 0.007 mol). The crude reaction mixture was purified by column chromatography on silica eluting with ethyl acetate: pentane (2:1) to give the title compound as a yellow oil that crystallised on standing (0.59 g, 0.002 mol, 35%). ¹H NMR (CDCl₃, 400 MHz) δ: 1.18 (t, 3H), 2.24 (q, 2H), 2.58 (bs, 1H), 3.34 (m, 1H), 3.63 (m, 1H), 3.88 (s, 3H), 4.82 (dd, 1H), 5.98 (bs, 1H), 6.82 (m, 1H), 7.01 (m, 2H). LRMS: m/z 242 (M−H⁺).

### Example 19

### 1-(4-Fluoro-3-methoxyphenyl)-2-propylaminoethanol

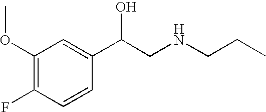

Prepared following the same method as for example 3 starting with the amide from example 18 (585 mg, 2.42 mmol). After refluxing in 6M HCl (aq) the reaction mixture was cooled and extracted with diethyl ether (2×50 ml). The organic layers were discarded and the aqueous layer basified by the addition of potassium carbonate. The aqueous residue was then extracted with ethyl acetate (3×50 ml). The organic extracts were combined and dried over anhydrous magnesium sulphate, filtered and concentrated in vacuo to give the title compound as a pale yellow oil (448 mg, 1.97 mmol, 81%). ¹H NMR (CD₃OD, 400 MHz) δ: 0.96 (t, 3H), 1.58 (m, 2H), 2.63 (m, 2H), 2.79 (d, 2H), 3.96 (s, 3H), 4.77 (t, 1H), 6.90 (m, 1H), 7.03 (t, 1H), 7.11 (d, 1H). LRMS: m/z 228 (M−H⁺).

### Example 20

### 2-Chloro-N-[2-(4-fluoro-3-methoxyphenyl-2-hydroxyethyl]-N-propylacetamide

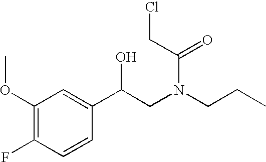

Prepared following the same method as for example 4 starting with the amine from example 19 (0.84 g, 4.00 mmol) to give the title compound as a yellow oil (0.97 g, 3.00 mmol, 87%). LRMS: m/z 304 (M−H⁺). This was taken on crude.

### Example 21

### 6-(4-Fluoro-3-methoxyphenyl)-4-propylmorpholin-3-one

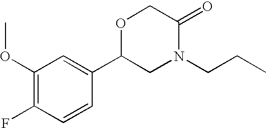

Prepared following the same method as for example 5 starting with the amide from example 20 (0.96 g, 3.00 mmol) to give the title compound as a yellow oil (0.64 g, 2.40 mmol, 75%). ¹H NMR (CDCl₃, 400 MHz) δ: 0.94 (t, 3H), 1.62 (m, 2H), 3.33 (m, 2H), 3.48 (m, 2H), 3.91 (s, 3H), 4.34 (d, 1H), 4.43 (d, 1H), 4.76 (dd, 1H), 6.85 (m, 1H), 7.01-7.08 (m, 2H). LRMS: m/z 268 (M−H⁺).

### Example 22

### 2-(4-Fluoro-3-methoxyphenyl)-4-propylmorpholine

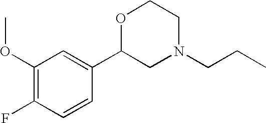

Prepared following the same method as for example 6 starting with the morpholin-3-one from example 21 (633 mg, 2.37 mmol). After refluxing in 6M HCl (aq) the reaction mixture was cooled and extracted with diethyl ether (2×20 ml). The organic layers were discarded and the aqueous layer basified by the addition of potassium carbonate. The aqueous residue was then extracted with ethyl acetate (3×20 ml). The organic extracts were combined and dried over anhydrous magnesium sulphate, filtered and concentrated in vacuo to give the title compound as a yellow oil (552 mg, 2.18 mmol, 92%). ¹H NMR (CD₃OD, 400 MHz) δ: 0.95 (t, 3H), 1.58 (m, 2H), 2.02 (t, 1H), 2.22 (dt, 1H), 2.38 (t, 2H), 2.85 (d, 1H), 2.93 (d, 1H), 3.80 (m, 1H), 3.84 (s, 3H), 4.01 (dd, 1H), 4.50 (dd, 1H), 6.88 (m, 1H), 7.02 (t, 1H), 7.09 (d, 1H). LRMS: m/z 254 (M−H⁺).

### Example 23a

### R-(+)-2-Fluoro-5-(4-propylmorpholin-2-yl)phenol

### Example 23b

### S-(−)-2-Fluoro-5-(4-propylmorpholin-2-yl)phenol

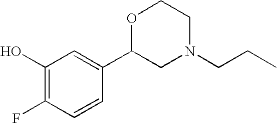

Prepared following the same method as for example 7 starting with the anisole from example 22 (200 mg, 0.789 mmol). The crude reaction mixture was purified by column chromatography on silica eluting with dichloromethane: methanol (90:10) to give the title racemic compound as a dark yellow viscous oil (149 mg, 0.62 mmol, 79%). The enantiomers were separated by chiral chromatography (Chiralpak AD 250*20 mm column) eluting with hexane: isopropyl alcohol: (90:10) to give enantiomer 1 (23a) as an opaque oil (15 mg) (ee>99.5%) and enantiomer 2 (23b) as a crystalline solid (16 mg) (ee>99%). Enantiomer 1 (23a): ¹H NMR (CD₃OD, 400 MHz) δ: 0.95 (t, 3H), 1.58 (m, 2H), 2.01 (t, 1H), 2.21 (dt, 1H), 2.37 (t, 2H), 2.82-2.97 (bq, 2H), 3.78 (dt, 1H), 3.99 (dd, 1H), 4.43 (d, 1H), 6.78 (m, 1H), 6.89-7.01 (m, 2H). LRMS: m/z 240 (M-H⁺). α_{D}=+0.91 (Ethanol 1.10 mg/ml). Enantiomer 2 (23b): ¹H NMR (CD₃OD, 400 MHz) δ: 0.96 (t, 3H), 1.58 (m, 2H), 2.01 (t, 1H), 2.22 (dt, 1H), 2.38 (t, 2H), 2.78 (dd, 2H), 3.78 (dt, 1H), 4.00 (dd, 1H), 4.43 (dd, 1H), 6.78 (m, 1H), 6.91 (d, 1H), 6.98 (t, 1H). LRMS: m/z 240 (M−H⁺). α_{D}=−0.40 (Ethanol 1.00 mg/ml).

### Example 24

### 2-Amino-1-(4-benzyloxyphenyl)ethanol

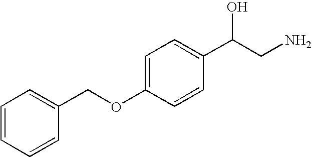

Potassium cyanide (20.15 g, 0.31 mol) and ammonium chloride (16.4 g, 0.31 mol) were dissolved in water (60 ml) to which was added 4-benzyloxybenzaldehyde (32.9 g, 0.155 mol) followed by diethyl ether (100 ml). The reaction mixture was stirred vigorously for 48 hours at room temperature before extracting with ethyl acetate (2×200 ml). The combined organic layers were dried over anhydrous magnesium sulphate, filtered and concentrated in vacuo to give the cyanohydrin intermediate as a yellow solid (34.2 g, 0.14 mol, 90%). The cyanohydrin was then dissolved in dry THF (300 ml) and borane-methyl sulphide complex (26.6 ml, 0.28 mol) was added. The reaction mixture was refluxed for 2 hours before being quenched with methanol (50 ml). Water (50 ml) was added followed by c.HCl (40 ml) and the reaction mixture was stirred for 2 hours until the exotherm subsided. The reaction mixture was then concentrated in vacuo and the residue diluted with water (100 ml). The aqueous solution was then basified by addition of NH₄OH (30 ml), and extracted with ethyl acetate (3×150 ml). The organic extracts were dried over anhydrous magnesium sulphate, filtered and concentrated in vacuo to give the title compound as a white solid (24.8 g, 0.10 mol, 73%). ¹H NMR (CDCl₃, 400 MHz) δ: 1.62 (bs, 3H), 2.81 (dd, 1H), 2.99 (d, 1H), 4.61 (q, 1H), 5.07 (s, 2H), 6.95 (d, 2H), 7.22-7.45 (m, 7H). LRMS: m/z 244 (M−H⁺).

### Example 25

### N-[2-(4-Benzyloxylphenyl)-2-hydroxyethyl)propionamide

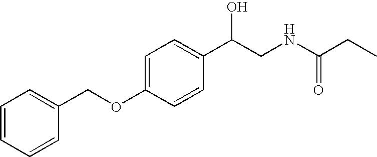

The amine from example 24 (24.8 g, 0.10 mol) was dissolved in dichloromethane (700 ml) and to this was added triethylamine (20.86 ml, 0.15 mol). The reaction mixture was stirred and cooled to 0° C., before propionyl chloride (7.12 ml, 0.082 mol) was added dropwise. The reaction mixture was then allowed to warm to room temperature over 16 hours before quenching with 3M HCl (aq) (20 ml) and water (100 ml). The reaction mixture was then extracted with dichloromethane (3×200 ml) and the combined organic layers dried over anhydrous magnesium sulphate, filtered and concentrated in vacuo to give the title compound as a clear viscous gum (27.5 g, 0.092 mol, 90%). ¹H NMR (CDCl₃, 400 MHz) δ: 1.10 (t, 3H), 2.19 (q, 2H), 3.32-3.43 (m, 4H), 4.81 (s, 2H), 5.11 (m, 1H), 6.99 (d, 2H), 7.25-7.42 (m, 7H). LRMS: m/z 298 (M−H⁻).

### Example 26

### 1-(4-benzyloxyphenyl)-2-propylaminoethanol

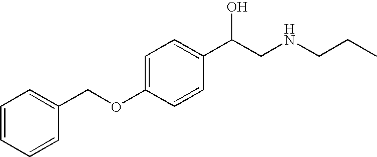

To the amide from example 25 (27.5 g, 0.092 mol) in dry THF (100 ml) was added borane-methyl sulphide complex (17.5 ml, 0.18 mol) and the reaction mixture was stirred at reflux for 2 hours. The reaction mixture was cooled then quenched with methanol (30 ml). Water (50 ml) and c.HCl (35 ml) were added and the reaction mixture stirred until all bubbling ceased before concentrating in vacuo. To the residue water (250 ml) was added, before basifying by addition of NH₄OH (30 ml). The aqueous layer was extracted with ethyl acetate (3×200 ml) and the combined organic extracts dried over anhydrous magnesium sulphate, filtered and concentrated in vacuo to give the title compound as a white solid (26.1 g, 0.09 mol, 99%). ¹H NMR (CD₃OD, 400 MHz) δ: 0.95 (t, 3H), 1.58 (q, 2H), 2.62 (m, 2H), 2.81 (m, 2H), 4.72 (dd, 1H), 5.05 (s, 2H), 6.95 (d, 2H), 7.24 (m, 3H), 7.35 (t, 2H), 7.41 (d, 2H). LRMS: m/z 286 (M−H⁺).

### Example 27

### 6-(4-benzyloxyphenyl)-4-propylmorpholin-3-one

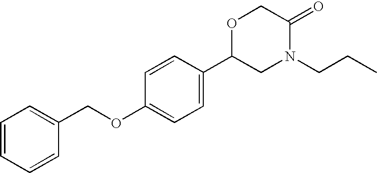

Sodium hydroxide (22.5 g, 0.56 mol) in water (100 ml) was added to the amine from example 26 (26.0 g, 0.09 mol) in dichloromethane (400 ml) and the solution vigorously stirred at room temperature. Chloroacetylchloride (8.6 ml, 0.11 mol) was then added and the reaction mixture stirred for a further 60 minutes. The layers were separated and the aqueous layer re-extracted with dichloromethane (200 ml). The organic extracts were combined, dried over anhydrous magnesium sulphate, filtered and concentrated in vacuo to give a colourless oil. Potassium hydroxide (15.0 g, 0.27 mol), isopropyl alcohol (400 ml) and the colourless oil residue were stirred together as an opaque solution with water (30 ml) for 2 hours. The reaction mixture was concentrated in vacuo and the yellow residue dissolved in ethyl acetate (200 ml). This was partitioned with water (200 ml) then brine (200 ml). The organic fraction was dried over anhydrous magnesium sulphate, filtered and concentrated in vacuo to give the title compound as a white solid (19.9 g, 0.06 mol, 67%). ¹H NMR (CDCl₃, 400 MHz) δ: 0.95 (t, 3H), 1.62 (m, 2H), 3.34 (m, 2H), 3.51 (m, 2H), 4.32 (d, 1H), 4.41 (d, 1H), 4.72 (dd, 1H), 5.04 (s, 2H), 6.98 (d, 2H), 7.31-7.43 (m, 7H). LRMS: m/z 326 (M−H⁺).

### Example 28

### 2-(4-benzyloxyphenyl)-4-propylmorpholine

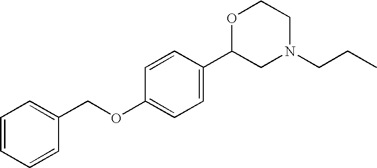

Prepared following the same method as for example 26 with the morpholin-3-one from example 27 (19.9 g, 0.061 mol) to give the title compound as a colourless oil (17 g, 0.055 mol, 90%). ¹H NMR (CDCl₃, 400 MHz) δ: 0.95 (t, 3H), 1.55 (q, 2H), 2.06 (t, 1H), 2.21 (dt, 1H), 2.35 (dd, 2H), 2.80 (d, 1H), 2.91 (d, 1H), 3.82 (dt, 1H), 4.02 (dd, 1H), 4.52 (dd, 1H), 5.05 (s, 2H), 6.98 (t, 2H), 7.24-7.42 (m, 7H). LRMS: m/z 312 (M−H⁺).

### Example 29

### 4-(4-Propylmorpholin-2-yl)phenol

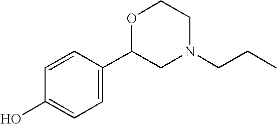

Benzyl ether from example 28 (3.0 g, 9.64 mmol) was dissolved in methanol (150 ml) and 10% palladium on charcoal (800 mg) was added. The reaction mixture was stirred for a few minutes before ammonium formate (6.17 g, 96.4 mmol) was added portionwise. The reaction mixture was carefully heated to 80° C. until gas evolution had ceased. After cooling, the reaction mixture was filtered through arbacel, washed with methanol (50 ml) and concentrated in vacuo to give the title compound as a white crystalline solid (1.51 g, 6.83 mmol, 71%). ¹H NMR (CDCl₃, 400 MHz) δ: 0.91 (t, 3H), 1.58 (q, 2H), 2.10 (t, 1H), 2.22 (t, 1H), 2.40 (dd, 2H), 2.81 (d, 1H), 2.93 (d, 1H), 3.85 (t, 1H), 4.02 (dd, 1H), 4.57 (d, 1H), 6.79 (d, 2H), 7.21 (d, 2H). LRMS: m/z 222 (M−H⁺).

### Example 30

### 2-Bromo-4-(4-propylmorpholin-2-yl)phenol

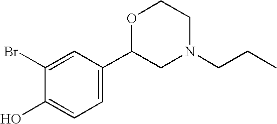

To the phenol from example 29 (200 mg, 0.9 mmol) in dichloromethane (5 ml) was added N-bromosuccinimide (161 mg, 0.9 mmol). The reaction mixture was stirred at room temperature for 55 hours, before concentrating in vacuo. The crude product was purified by column chromatography on silica eluting with dichloromethane: methanol (95:5) to give the title compound as a white foam (117.5 mg, 0.39 mmol, 44%). ¹H NMR (CDCl₃, 400 MHz) δ: 0.96 (t, 3H), 1.59 (q, 2H), 2.03 (t, 1H), 2.23 (t, 1H), 2.40 (t, 2H), 2.81 (d, 1H), 2.98 (d, 1H), 3.82 (t, 1H), 4.01 (d, 1H), 4.56 (d, 1H), 6.96 (d, 1H), 7.20 (d, 1H), 7.49 (s, 1H). LRMS: m/z 302 (M−H⁺, Br isotope).

### Example 31

### 2-(4-benzyloxy-3-bromophenyl)-4-propylmorpholine

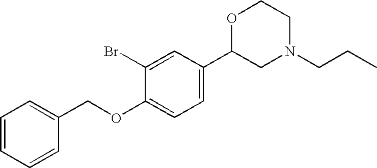

To the phenol from example 30 (117.5 mg, 0.39 mmol) in dry DMF (10 ml), under an atmosphere of nitrogen, was added potassium carbonate (75 mg, 0.54 mmol) and benzyl bromide (0.07 ml, 0.54 mmol). The reaction mixture was heated to 150° C. for 48 hours. After cooling, the reaction mixture was concentrated in vacuo and the residue partitioned between ethyl acetate (50 ml) and water (50 ml). The aqueous layer was then re-extracted with ethyl acetate (2×20 ml). The combined organic extracts were then dried over anhydrous magnesium sulphate, filtered and concentrated in vacuo to give the crude product as a brown oil. This was purified by column chromatography on silica eluting with dichloromethane: methanol (98:2) to give the title compound as a colourless oil (153 mg, 0.39 mmol, 100%). ¹H NMR (CDCl₃, 400 MHz) δ: 0.93 (t, 3H), 1.56 (q, 2H), 2.05 (t, 1H), 2.25 (t, 1H), 2.37 (t, 2H), 2.82 (d, 1H), 2.92 (d, 1H), 3.85 (t, 1H), 4.02 (d, 1H), 4.52 (d, 1H), 5.15 (s, 2H), 6.87 (d, 1H), 7.20 (d, 1H), 7.30 (d, 1H), 7.37 (t, 2H), 7.45 (d, 2H), 7.58 (s, 1H). LRMS: m/z 392 (M−H⁺).

### Example 32

### 2-Benzyloxy-5-(4-propylmorpholin-2-yl)benzoic acid methyl ester

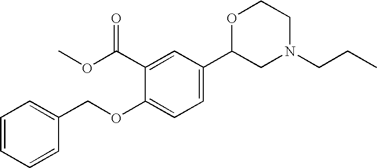

To the bromide from example 31 (153 mg, 0.39 mmol) in dry DMF (4 ml) was added triethylamine (2.1 ml, 0.78 mmol) and methanol (2 ml) and the reaction mixture stirred for 5 minutes. [1,1′-Bis(diphenylphosphino)ferrocene]dichloropalladium (II), complex with dichloromethane (1:1) (16 mg, 0.02 mmol) was added before carbon monoxide (g) (3 inflated balloons) was bubbled through the reaction mixture. The reaction mixture was then heated to 100° C. for 16 hours under an atmosphere of carbon monoxide. After cooling, the reaction mixture was concentrated in vacuo and the residue partitioned between ethyl acetate (25 ml) and water (20 ml). The organic layer was separated, washed with brine (20 ml) and dried over anhydrous magnesium sulphate, filtered and concentrated in vacuo to give a black solid. Purification by column chromatography on silica eluting with dichloromethane: methanol: ammonia (90:10:1) gave the title compound as a colourless oil (105 mg, 0.28 mmol, 73%). ¹H NMR (CDCl₃, 400 MHz) δ: 0.94 (t, 3H), 1.60 (m, 2H), 2.18 (s, 4H), 2.43 (m, 2H), 3.00 (m, 2H), 3.90 (s, 3H), 4.04 d, 1H), 5.18 (s, 2H), 5.97 (d, 1H), 7.26-7.47 (m, 6H), 7.82 (s, 1H). LRMS: m/z 370 (M−H⁺).

### Example 33

### 2-Benzyloxy-5-(4-propylmorpholin-2-yl)benzoic acid

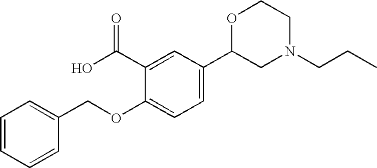

To the methyl ester from example 32 (105 mg, 0.28 mmol) in methanol (5 ml) was added 10% sodium hydroxide (aq) (15 ml) and the milky white suspension was refluxed for 2 hours. The now colourless reaction mixture was cooled then neutralised by addition of 2M HCl (aq) (few drops). The reaction mixture was then concentrated in vacuo to give the title compound as an off-white solid (99 mg, 0.28 mmol, 100%). LRMS: m/z 355 (M−H⁺). This material was taken on crude to example 34.

### Example 34

### 2-Benzyloxy-5-(4-propylmorpholin-2-yl)benzamide

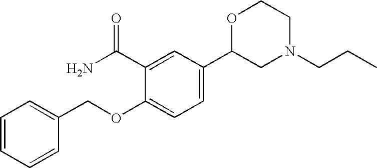

To the crude benzoic acid from example 33 (99 mg, 0.28 mmol) was added thionyl chloride (5 ml) and the reaction mixture heated to 50° C. for 2 hours. The reaction mixture was cooled and the excess thionyl chloride was removed in vacuo. The residue was then dissolved in dichloromethane (10 ml) and ammonia (g) was bubbled through the reaction mixture for 10 minutes. The resulting suspension was stirred at room temperature for 1 hour before concentrating in vacuo. The crude material was purified by column chromatography on silica eluting with dichloromethane: methanol: ammonia (95:5:0.5) to give the title compound as an off-white solid (88 mg, 0.25 mmol, 90%). ¹H NMR (CDCl₃, 400 MHz) δ: 0.94 (t, 3H), 1.59 (m, 2H), 2.15-2.42 (m, 4H), 2.87 (m, 1H), 3.03 (m, 1H), 3.96 (m, 1H), 4.02 (d, 1H), 4.67 (m, 1H), 5.19 (s, 2H), 5.72 (m, 1H), 7.04 (d, 1H), 7.41 (m, 5H), 7.50 (d, 1H), 7.70 (m, 1H), 8.21 (s, 1H). LRMS: m/z 355 (M−H⁺).

### Example 35

### 2-Hydroxy-5-(4-propylmorpholin-2-yl)benzamide

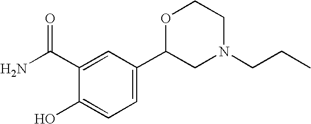

Prepared using the same method as for example 29 with the benzyl ester from example 34 (80 mg, 0.22 mmol) to give the title compound as an off-white solid (56 mg, 0.21 mmol, 96%). ¹H NMR (CD₃OD, 400 MHz) δ: 0.95 (t, 3H), 1.55 (m, 2H), 2.13 (t, 1H), 2.29 (t, 1H), 2.42 (m, 2H), 2.88 (d, 1H), 2.97 (d, 1H), 3.81 (t, 1H), 4.00 (d, 1H), 4.49 (d, 1H), 6.87 (d, 1H), 7.42 (d, 1H), 7.78 (s, 1H). LRMS: m/z 265 (M−H⁺).

### Example 36

### 2-Nitro-4-(4-propylmorpholin-2-yl)phenol

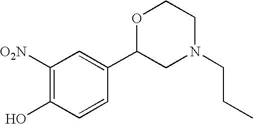

The phenol from example 29 (100 mg, 0.45 mmol) was dissolved in nitric acid:water (1:3) (2 ml) and stirred at room temperature for 10 minutes. The reaction mixture was then diluted with water (5 ml) and basified with NH₄OH (1 ml), before extracting into ethyl acetate (3×10 ml). The organic extracts were combined and dried over anhydrous magnesium sulphate, filtered and concentrated in vacuo to give the title compound as a yellow solid (95 mg, 0.35 mmol, 79%). ¹H NMR (CDCl₃, 400 MHz) δ: 0.97 (t, 3H), 1.33 (t, 2H), 1.43-1.79 (bm, 4H), 2.02 (d, 3H), 4.06 (m, 2H), 7.17 (d, 1H), 7.60 (d, 1H), 8.16 (s, 1H), 10.55 (bs, 1H). LRMS: m/z 267 (M−H+).

### Example 37

### 2-Amino-4-(4-propylmorpholin-2-yl)phenol

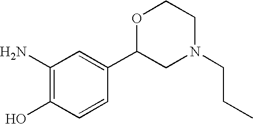

To the nitro from example 36 (95 mg, 0.35 mmol) in ethanol (10 ml) was added 10% palladium on charcoal (50 mg) and ammonium formate (100 mg, XS). The reaction mixture was gently heated to 70° C. and held at this temperature for 1 hour before it was allowed to cool to room temperature. The reaction mixture was then filtered through arbacel and washed with ethanol (20 ml) then dichloromethane (20 ml). The organic washes were combined and concentrated in vacuo to give the title compound as a yellow solid (65 mg, 0.28 mmol, 78%). ¹H NMR (CDCl₃, 400 MHz) δ: 0.91 (t, 3H), 1.55 (m, 2H), 2.12 (t, 1H), 2.25 (dt, 1H), 2.40 (t, 2H), 2.81-2.92 (dd, 2H), 3.82 (t, 1H), 4.00 (d, 1H), 4.42 (d, 1H), 6.60 (m, 2H), 6.71 (s, 1H). LRMS: m/z 237 (M−H⁺).

### Example 38

### 5-Bromo-2-(2,5-dimethylpyrrol-1-yl pyridine

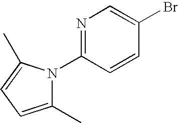

5-Bromopyridin-2-yl-amine (13.8 g, 0.08 mol), acetonylacetone (14.1 ml, 0.12 mol) and p-toluenesulphonic acid (100 mg) were dissolved in toluene (180 ml) and refluxed under Dean Stark conditions for 14 hours. After cooling, the brown solution was poured into water (200 ml) and extracted with toluene (2×200 ml). The organic extracts were combined and washed with brine (50 ml) then dried over anhydrous magnesium sulphate, filtered and concentrated in vacuo to give crude product. This was purified by column chromatography on silica eluting with ethyl acetate: pentane (1:3) to give the title compound as a brown oil (18.4 g, 0.073 mol, 92%). ¹H NMR (CDCl₃, 400 MHz) δ: 2.18 (s, 6H), 5.90 (s, 2H), 7.11 (d, 1H), 7.92 (d, 1H), 8.62 (s, 1H). LRMS: m/z 253 (M−H⁺, Br isotope).

### Example 39

### 2-Chloro-1-[6-(2,5-dimethylpyrrol-1-yl)pyridin-3-yl]ethanone

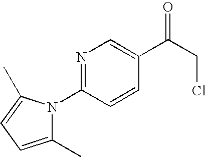

To a solution of bromo pyridine from example 38 (2 g, 8.0 mmol) at −78° C., in dry THF (30 ml), was added butyllithium (2.5M in hexanes) (3.5 ml 8.8 mmol), dropwise over 20 minutes. The reaction mixture was stirred for 30 minutes then 2-chloro-N-methoxy-N-methylacetamide (1.2 g, 8.8 mmol) in dry THF (20 ml) was added dropwise keeping the temperature at −78° C. Stirring was continued for 30 minutes at this temperature before 1M HCl (aq) (50 ml) was added and the reaction mixture warmed to room temperature. The organic layer was separated and the aqueous layer washed with ethyl acetate (50 ml). The organic layers were combined then washed with 3M NaOH (aq) (10 ml) and brine (10 ml) before being dried over anhydrous magnesium sulphate, filtered and concentrated in vacuo to give crude title compound as a brown oil (1.34 g, 5.4 mmol, 67%). ¹H NMR (CDCl₃, 400 MHz) δ: 2.20 (s, 6H), 4.68 (s, 2H), 5.92 (s, 2H), 7.32 (d, 1H), 8.38 (d, 1H), 9.16 (s, 1H). LRMS: m/z 249 (M−H⁺).

### Example 40

### 2-(2,5-dimethylpyrrol-1-yl)-5-oxiranylpyridine

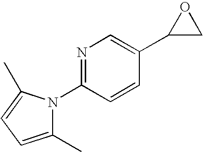

To the ketone from example 39 (1.34 g, 5.4 mmol) dissolved in dry THF (20 ml), cooled to 0° C., was added sodium borohydride (308 mg, 8.1 mmol) portionwise. The reaction mixture was stirred for 2 hours then 3M NaOH (aq) (10 ml) was added and stirring continued for a further 16 hours. The reaction mixture was extracted with ethyl acetate (2×20 ml) and the combined organic extracts washed with brine (5 ml), dried over anhydrous magnesium sulphate, filtered and concentrated in vacuo. The residue was purified by column chromatography on silica eluting with ethyl acetate: pentane (1:5) to give the title compound as a colourless oil (900 mg, 4.2 mmol, 78%). ¹H NMR (CDCl₃, 400 MHz) δ: 2.13 (s, 6H), 2.91 (dd, 1H), 3.25 (t, 1H), 3.98 (t, 1H), 5.90 (s, 2H), 7.20 (d, 1H), 7.62 (dd, 1H), 8.58 (s, 1H). LRMS: m/z 215 (M−H⁺).

### Example 41

### 1-[6-(2,5-dimethylpyrrol-1-yl]pyridin-3-yl-2-propylaminoethanol

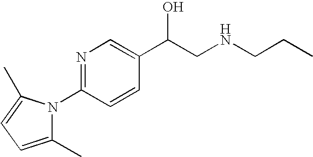

To the epoxide from example 40 (900 mg, 4.2 mmol) in DMSO (5 ml) was added propylamine (4 ml, 4.8 mmol) and the reaction mixture was heated to 40° C. for 4 days. The reaction mixture was then cooled and 3M HCl (aq) (10 ml) and water (10 m[) were added before washing with diethyl ether (2×10 ml). This organic layer was discarded. The aqueous layer was basified with NH₄OH (5 ml) and extracted with ethyl acetate (3×10 ml). The organic extracts were combined and dried over anhydrous magnesium sulphate, filtered and concentrated in vacuo to give the title compound as an oil (1.15 g, 4.2 mmol, 100%). ¹H NMR (CDCl₃, 400 MHz) δ: 0.93 (t, 3H), 1.62 (m, 2H), 2.11 (s, 6H), 2.69-2.82 (m, 3H), 3.06 (dd, 1H), 3.60 (bs, 2H), 4.92 (dd, 1H), 5.84 (s, 2H), 7.20 (d, 1H), 7.88 (d, 1H), 8.61 (s, 1H). LRMS: m/z 274 (M−H⁺).

### Example 42

### 6-[6-(2,5-dimethylpyrrol-1-yl)pyridin-3-yl]-4-propylmorpholi-3-one

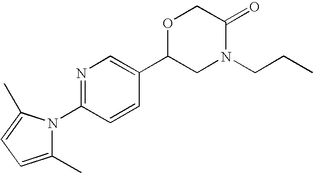

Prepared following the same method as for example 27 with the amine from example 41 (1.15 g, 4.2 mmol). Purification by column chromatography on silica eluting with dichloromethane: methanol (98:2) gave the title compound as a brown film (191 mg, 0.61 mmol, 14%). ¹H NMR (CDCl₃, 400 MHz) δ: 0.97 (t, 3H), 1.65 (m, 2H), 2.13 (s, 6H), 3.38 (m, 1H), 3.42-3.56 (m, 2H), 6.61 (t, 1H), 4.35 (d, 1H), 4.45 (d, 1H), 4.91 (dd, 1H), 6.91 (s, 2H), 7.22 (d, 1H), 7.89 (d, 1H), 8.61 (s, 1H). LRMS: m/z 314 (M−H⁺).

### Example 43

### 6-[6-(2,5-dimethylpyrrol-1-yl)pyridin-3-yl]-4-propylmorpholine

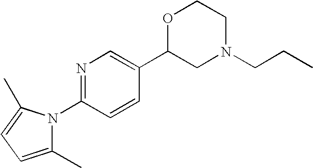

To a solution of the morpholin-3-one from example 42 (191 mg, 0.61 mmol) in dry THF (5 ml) was added lithium aluminium hydride (1M solution in diethyl ether) (1.25 ml, 0.61 mmol) and the reaction mixture was warmed to reflux for 2.5 hours. The reaction mixture was cooled to room temperature then 1M NaOH (1.25 ml) was added to give a white precipitate. The reaction mixture was filtered and concentrated in vacuo. The white solid was discarded. The concentrated filtrate was purified by column chromatography on silica eluting with dichloromethane: methanol (95:5) to give the title compound as a white film (108 mg, 0.36 mmol, 59%). ¹H NMR (CDCl₃, 400 MHz) δ: 0.92 (t, 3H), 1.61 (q, 2H), 2.10 (s, 6H), 2.15 (m, 1H), 2.29 (dt, 1H), 2.40 (t, 2H), 2.82 (d, 1H), 3.02 (d, 1H), 3.90 (t, 1H), 4.08 (d, 1H), 4.71 (d, 1H), 5.89 (s, 2H), 7.20 (d, 1H), 7.81 (d, 1H), 8.60 (s, 1H). LRMS: m/z 300 (M−H⁺).

### Example 44

### BP-897

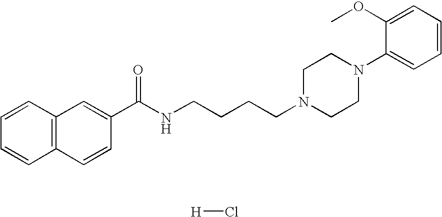

BP-897 is a dopamine D3 receptor agonist which is under development by Bioprojet for the potential treatment of drug craving and vulnerability to relapse that are elicited by drug-associated environmental stimuli. BP-897 entered phase II evaluation for drug dependence in November 2000; these trials were ongoing in November 2001.

BP-897 has been shown to inhibit forskolin-induced cAMP accumulation in NG 108-15 cells expressing the D3 receptor. This response was completely inhibited by haloperidol. In these cells, BP-897 was also shown to increase mitogenesis, a D3-mediated receptor response, this effect was antagonized by nafadotride.

BP-897 has been shown to be a high affinity and potent D3 receptor agonist (Ki=0.9 nM), but a weak D2 receptor antagonist (Ki=61 nM).

Biology Examples

1.0 Methods

1.1. Animal Test Method

1.1.1 Male and Female Anaesthetised Rabbit Methodology

Male and female New Zealand rabbits (˜2.5 kg) were pre-medicated with a combination of Medetomidine (Domitor®) 0.5 ml/kg i.m., and Ketamine (Vetalar®) 0.25 ml/kg i.m. whilst maintaining oxygen intake via a face mask. The rabbits were tracheotomised using a Portex™ uncuffed endotracheal tube 3 ID., connected to ventilator and maintained at a ventilation rate of 30-40 breaths per minute, with an approximate tidal volume of 18-20 ml, and a maximum airway pressure of 10 cm H₂O. Anaesthesia was then switched to Isoflurane and ventilation continued with O₂ at 2 l/min. The right marginal ear vein was cannulated using a 23 G or 24 G catheter, and Lactated Ringer solution perfused at 0.5 ml/min. The rabbit was maintained at 3% Isoflurane during invasive surgery, dropping to 2% for maintenance anaesthesia. The left jugular vein was exposed, isolated and then cannulated with a PVC catheter (17 G) for the infusion of drugs and compounds.

The left groin area of the rabbit was shaved and a vertical incision was made approximately 5 cm in length along the thigh. The femoral vein and artery were exposed, isolated and then cannulated with a PVC catheter (17 G) for the infusion of drugs and compounds. Cannulation was repeated for the femoral artery, inserting the catheter to a depth of 10 cm to ensure that the catheter reached the abdominal aorta. This arterial catheter was linked to a Gould system to record blood pressure. Samples for blood gas analysis were also taken via the arterial catheter. Systolic and diastolic pressures were measured, and the mean arterial pressure calculated using the formula (diastolic×2+systolic)÷3. Heart rate was measured via the pulse oxymeter and Po-ne-mah data acquisition software system (Ponemah Physiology Platform, Gould Instrument Systems, Inc.).

A ventral midline incision was made into the abdominal cavity. The incision was about 5 cm in length just above the pubis. The fat and muscle was bluntly dissected away to reveal the hypogastric nerve which runs down the body cavity. It was essential to keep close to the side curve of the pubis wall in order to avoid damaging the femoral vein and artery which lie above the pubis. The sciatic and pelvic nerves lie deeper and were located after further dissection on the dorsal side of the rabbit. Once the sciatic nerve is identified, the pelvic nerve was easily located. The term pelvic nerve is loosely applied; anatomy books on the subject fail to identify the nerves in sufficient detail. However, stimulation of the nerve causes an increase in vaginal and clitoral blood flow in females and in intracavernosal pressure and cavernosal blood flow in males, and innervation of the pelvic region. The pelvic nerve was freed away from surrounding tissue and a Harvard bipolar stimulating electrode was placed around the nerve. The nerve was slightly lifted to give some tension, then the electrode was secured in position. Approximately 1 ml of light paraffin oil was placed around the nerve and electrode. This acts as a protective lubricant to the nerve and prevents blood contamination of the electrode. The electrode was connected to a Grass S88 Stimulator. The pelvic nerve was stimulated using the following parameters:—5V, pulse width 0.5 ms, duration of stimulus 20 seconds with a frequency of 16 Hz. Reproducible responses were obtained when the nerve was stimulated every 15-20 minutes. Several stimulations using the above parameters were performed to establish a mean control response. The compound(s) to be tested were infused, via the jugular vein, using a Harvard 22 infusion pump allowing a continuous 15 minute stimulation cycle.

In males, the skin and connective tissue around the penis was removed to expose the penis. A catheter set (Insyte-W, Becton-Dickinson 20 Gauge 1.1×48 mm) was inserted through the tunica albica into the left corpus cavernosal space and the needle removed, leaving a flexible catheter. This catheter was linked via a pressure transducer (Ohmeda 5299-04) to a Gould system to record intracavernosal pressure. Once an intracavernosal pressure was established, the catheter was sealed in place using Vetbond (tissue adhesive, 3M). Heart rate was measured via the pulse oxymeter and Po-ne-mah data acquisition software system (Ponemah Physiology Platform, Gould Instrument Systems, Inc.).

Intracavernosal blood flow was recorded either as numbers directly from the Flowmeter using Po-ne-mah data acquisition software (Ponemah Physiology Platform, Gould Instrument Systems, Inc.), or indirectly from Gould chart recorder trace. Calibration was set at the beginning of the experiment (0-125 ml/min/100 g tissue).

In females, a ventral midline incision is made, at the caudal end of the pubis, to expose the pubic area. Connective tissue is removed to expose the tunica of the clitoris, ensuring that the wall is free from small blood vessels. The external vaginal wall is also exposed by removing any connective tissue. One Laser Doppler flow probe is inserted 3 cm into the vagina, so that half the probe shaft is still visible. A second probe is positioned so that it lay just above the external clitoral wall. The position of these probes is then adjusted until a signal is obtained. A second probe is placed just above the surface of the blood vessel on the external vaginal wall. Both probes are clamped in position.

Vaginal and clitoral blood flow is recorded either as numbers directly from the Flowmeter using Po-ne-mah data acquisition software (Ponemah Physiology Platform, Gould Instrument Systems, Inc.), or indirectly from Gould chart recorder trace. Calibration is set at the beginning of the experiment (0-125 ml/min/100 g tissue).

The selective dopamine D3 agonist was made up in saline+10% 1M NaOH.

Phosphodiesterase type 5 (PDE5) inhibitor may be made up in saline+5% 1M HCl. The agonists (and, where applicable, inhibitors) and vehicle controls were infused at a rate of 0.1 ml/second. Selective dopamine D3 agonists and, where applicable, PDE_{cAMP} inhibitors were left for 15 minutes prior to pelvic nerve stimulation.

All data are reported as mean±s.e.m. Significant changes were identified using Student's t-tests.

2.0 Results and Discussion

The examples hereinafter demonstrate that activating or enhancing the activity of a D3 dopamine receptor alone, i.e. without activating or enhancing the activity of D2 dopamine receptors or significantly activating or enhancing the activity of D2 dopamine receptors, results in penile erection and/or enhanced female genital blood flow.

In addition, the results hereinafter demonstrate that activation or enhancing the activity of D2 dopamine receptors is primarily responsible for adverse side effects observed following administration of non-selective dopamine agonists. The results demonstrate that D3 receptors are not responsible for, or not primarily responsible for, said side effects.

The results hereinafter further demonstrate that degree of selectivity of the agonist between D3 receptors and D2 receptors is important. Only selective D3 receptor agonists with a functional selectivity towards D3 receptors over D2 receptors which is at least about 3-times that achieved by pramipexole are sufficiently selective to result in the desired penile erection/female genital blood flow, whilst overcoming the side effects observed with non-selective dopamine agonists.

3.0 Compounds Used in Following Examples

3.1 7-OH-DPAT is a commercially available D3/D2 non-selective agonist.

3.2 Pramipexole (SND 919; 2-amino-4,5,6,7-tetrahydro-6-propylamino-benzthiazole-dihydrochloride) is a commercially available D3-preferring D3/D2 agonist, with a preference for D3 over D2 receptors of about 5-fold to about 9-fold.

3.3 Ropinirole is a commercially available balanced D3/D2 agonist, with a preference for D3 over D2 receptors of about 2-fold.

3.4 Trans-[4-[(4-Phenylpiperazin-1-yl)methyl]cyclohexyl]-pyrimidin-2-ylamine (PD-163,404) is a selective D2 agonist—see Wustrow et al. J. Med. Chem. 41, 760-771 (1998)—example 23b.

3.5 L-741,626 is a commercially available D2 antagonist (16-fold selective)—see Kulagowski et al. J. Med. Chem. 1996, May 10, 39 (10):1941-2 and Bowery et al. Br. J. Pharmacol. 1996, December: 119 (7): 1491-7.

3.6 SB-277,011 is a D3 antagonist (126-fold selective)—see Stemp et al. J. Med. Chem. (2000) 45, 1878-1885.

3.7 R-(−)-3-(4-Propylmorpholin-2-yl)phenol hydrochloride (see Example 8 of the “Chemistry Examples” section supra) is a selective D3 receptor agonist in accordance with the present invention.

3.8 S32504 is a preferential or selective agonist for dopamine D3 receptors as compared with dopamine D2 receptors. S32504 was said to possess anti-Parkinson and antidepressant properties. S32504 is 251-fold selective for D3 receptors over D2 receptors when analysed using a GTPγS assay. The chemical structure of S32504 is: 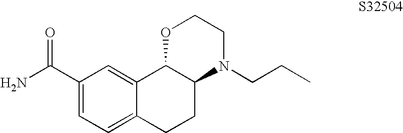
The compound S32504 was disclosed at American Chemical Society, 222^{nd} ACS National Meeting, Chicago, Ill. on 26-30 August 2001. (See Peglion et al. [abstract 208 of the proceedings]). The synthesis of this compound is described in EP 0 899 267 and also in Millan M. J. et al. Soc. Neurosci. Abst. (1999) 26. Pt. 2. Abs 588.2.

3.9 PNU-95666 (aka U-95666, U-95666A, PNU-95666E, sumanirole) ((R)-5,6-dihydro-N,N-dimethyl-4H-imidazo4,5,1-ij)quinolin-5-amine(R)-3] is claimed to be a selective D2 agonist (assessed by binding affinity) yet functionally is a non-selective D2/D3 dopamine agonist. The chemical structure of PNU-95666 is shown below:

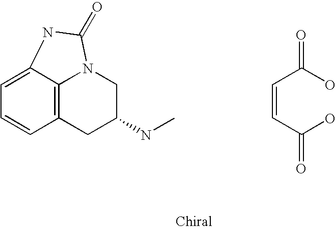

### Example 45

### Both D3 and D2 Receptors Involved in Induction/Maintenance of Penile Erection and Female Genital Blood Flow

Erectile responses were recorded by measuring intracavernosal pressure and/or clitoral/vaginal blood flow using surgically implanted telemetric device. The specific details of the surgical procedures, data acquisition and analysis can be found in detail in Bernabe J, Rampin O, Sachs B D, Giuliano F. Intracavernous pressure during erection in rats: an integrative approach based on telemetric recording. Am. J. Physiol. 1999 February; 276(2 Pt 2):R441-9.

Both D2 and D3 receptors are involved in the induction and maintenance of penile erection.

Apomorphine, 7-OH-DPAT and pramipexole all induce/enhance proerectile effects in male models of erection.

Mechanism of action studies have shown that 7-OH-DPAT (a D2/D3 non-selective agonist) and pramipexole (a D3-preferring D2/D3 agonist) can enhance the erectile mechanisms via activation of D3 or D2 receptors.

Pramipexole (0.1 μg/kg administered subcutaneously [s.c.]) induces a proerectile effect in telemetry rats.

The erectogenic effects of pramipexole (0.1 μg/kg s.c.) are observed in the presence of a selective D2 receptor antagonist (L-741,626 2 mg/kg s.c.) i.e. pramipexole erections are mediated by D3 receptor activation.

The erectogenic effects of pramipexole (0.1 μg/kg s.c.) are observed in the presence of a selective D3 receptor antagonist (SB-277,011 3 mg/kg s.c.) i.e. pramipexole erections are mediated by D2 receptor activation.

Concomitant application of L-741,626 and SB-277,011 (at 2 mg/kg s.c. and 3 mg/kg s.c., respectively) abolishes all pramipexole-induced erectile activity i.e. pramipexole-induced erections are mediated via D2 or D3 receptor activation.

No of erectogenic events/15 min

| | | | pramipexole + |
|---|---|---|---|
| | | | L-741626/ |
| | pramipexole + | pramipexole + | SB-277011 |
| Pramipexole | L-741626 | SB-277011 | concomitant |
| (0.1 μg/kg | D2 antagonist | D3 antagonist | D2/D3 |
| s.c.) | (2 mg/kg sc) | (3 mg/kg sc) | antagonists |
| 2.8 ± 0.2 n = 5 | 2.8 ± 0.6 n = 10 | 2.3 ± 0.4 n = 10 | 0.4 ± 0.4 n = 5 |

7-OH-DPAT (10 μg/kg s.c.) induces a proerectile effect in telemetry rats in the presence of a selective D2 receptor antagonist (L-741,626 2 mg/kg s.c.) or a selective D3 receptor antagonist (SB-277,011 3 mg/kg s.c.).

Concomitant application of L-741,626 and SB-277,011 at 2 mg/kg s.c. and 3 mg/kg s.c., respectively) abolishes all 7-OH-DPAT-induced erectile activity, i.e. 7-OH-DPAT-induced erections are mediated via D2 or D3 receptor activation.

Trans-[4-[(4-Phenylpiperazin-1-yl)methyl]cyclohexyl]-pyrimidin-2-ylamine, a selective D2 agonist, produced erections in rats. A selective D2 antagonist (L-741,626; 2 mg/kg s.c.) abolished trans-[4-[(4-Phenylpiperazin-1-yl)methyl]cyclohexyl]-pyrimidin-2-ylamine-induced erection. D2-mediated pathways are inhibited in studies investigating the role of D3 in pramipexole- and 7-OH-DPAT-induced erections.

R-(−)-3-(4-Propylmorpholin-2-yl)phenol hydrochloride (10 μg/kg s.c.), a selective D3 agonist, produced 2.25±0.25 erections/10 minutes in the telemetry rat.

This data strongly suggests that both D2 and D3 receptors are involved in the induction and maintenance of penile erection. A selective D3 agonist with selectivity over D2 receptors will provide an opportunity to increase the therapeutic ratio (therapeutic window; therapeutic index; therapeutic profile) between efficacy in treatment and side effects.

### Example 46

### Selective Activation of Dopamine D2 Receptors Induces Emesis in the Anaesthetised Dog

Male beagles, 11-17 kg were used. Following an overnight fast, animals were anaesthetised with chloralose (80 mg kg⁻¹ i.v.), having first been injected with an anaesthetic dose of the short acting barbiturate, thiopentone (15 mg kg⁻¹ i.v.). Following tracheal intubation, which ensured a patent airway even during the expulsion phase of the emetic response, the left femoral vein and artery were cannulated for further administration of anaesthetic and for recording haemodynamic parameters, respectively. When surgery was complete, surgical wounds were infiltrated with a long-acting local anaesthetic (bupivacaine) and animals were allowed to stabilise for 120 min before dosing with test compound. During the stabilisation period, respiratory rate was monitored to ensure that central respiratory centres were not deeply depressed, since the CNS efferent component of the emetic reflex is essentially conveyed via respiratory motor neurones. Once the animal had stabilised and respiratory rate was regular the first dose of the test compound was administered via subcutaneous (s.c.) injection in the dorsal surface of the neck. Animals were observed closely for 30 min post-dose and motor (e.g. swallowing, eye blinking, limb movements, changes in respiratory rate and depth, retching and vomiting) and haemodynamic changes noted and recorded. Escalating doses of apomorphine, pramipexole, trans-[4-[(4-Phenylpiperazin-1-yl)methyl]cyclohexyl]-pyrimidin-2-ylamine and R-(−)-3-(4-Propylmorpholin-2-yl)phenol hydrochloride (3, 10, 30, 100, 300, 1000 and 3000 μg kg⁻¹) were administered 60 to 90 minutes after the preceding dose, until a marked emetic response was observed. Blood samples were taken 15 min after administration of each dose of test compound (in animals which retched, the latency from injection of test drug to first retch was normally 7 to 13 min; see Table 1), and plasma samples frozen for later analysis. At the end of an experiment animals were sacrificed with an overdose of pentobarbitone.

Observations made on individual animals were used to determine the percentage of animals that retched when injected with a particular dose level of test compound. Percentage of animals retching was then used to construct a dose response curve and an ED₅₀ value determined from the appropriate curve.

Apomorphine—induced a marked emetic response in the chloralose anaesthetised dog. The emetic response was dose related, inducing retching in 0, 25 and 100% of animals dosed at 8.5, 26 and 85 μg kg⁻¹ s.c. (n=4), respectively. Group mean (±s.e.) number of retches was also dose related with 0.0, 2.5 L+2.5) and 28.0 L+7.5) retches at 8.5, 26 and 85 μg kg⁻¹ sc., respectively. Based on percentage of animals retching, apomorphine has an ED₅₀ for induction of the emetic reflex in the anaesthetised dog of 33 μg kg⁻¹ s.c.

Trans-[4-[(4-Phenylpiperazin-1-yl)methyl]cyclohexyl]-Pyrimidin-2-ylamine—Emesis was observed at 100 μg/kg during the study and the average free concentration of the corresponding sample is 3.6 nM. The EC₅₀'s for trans-[4-[(4-Phenylpiperazin-1-yl)methyl]cyclohexyl]-pyrimidin-2-ylamine are 3.8 nM=D2 and D3=>100 nM. The samples show that free concentrations were above the D2 EC₅₀ and well below the D3 EC₅₀ and so the emesis observed is likely to be due to a D2-mediated effect.

Pramipexole—Emesis was observed at 30 μg/kg during the study and the average free concentration of the corresponding sample was 3.0 nM. The EC₅₀'s for pramipexole are 2.75 nM=D2 and 0.56 nM=D3. Due to the free concentration in the appropriate sample being so close to both the D2 and D3 EC₅₀'s the conclusion whether the emesis observed is caused by a D2 or D3 effect cannot be made for this compound.

R-(−)-3-(4-Propylmorpholin-2-yl)phenol hydrochloride—No emesis was observed at doses up to 3000 μg/kg during the study. Free plasma concentration of the corresponding samples was 2700 nM which is approximately 270-fold greater than the D3 EC₅₀ for R-(−)-3-(4-Propylmorpholin-2-yl)phenol hydrochloride.

Assuming the concentrations measured represent the Cₘₐₓ and the pharmacokinetic profile follows the pharmacodynamic profile of emesis, the data suggests that the emesis observed is due to a D2-mediated effect based upon the trans-[4-[(4-Phenylpiperazin-1-yl)methyl]cyclohexyl]-pyrimidin-2-ylamine data. Both apomorphine and pramipexole are inconclusive in confirming the possible D2-mediated emesis observed.

This data strongly suggests that D2 receptors are involved in the induction emesis. A selective D3 agonist with selectivity over D2 receptors will provide an opportunity to increase the therapeutic ratio (therapeutic window; therapeutic index; therapeutic profile) between efficacy in treatment and side effects.

### Example 47

### D2 Dopamine Receptors Mediate the Pro-Emetic Effects of 7-OH-DPAT

Emesis induced in ferrets by the dopamine D2/D3 receptor agonist R(+)-7-OH-DPAT, can be blocked by the D2/D3 receptor antagonist, (S)-eticlopride (Yoshikawa et al., (1996) Eur. J. Pharmacol. 301 143-149). To better understand the mechanism for this effect, we compared the activity of (S)-eticlopride with the reported D3-selective antagonist, GR218231 (Murray et al., (1996) Bioorg. & Med. Chem. Letts. 6 403-408) and the D2 receptor-preferring antagonist, L-741,626 (Bowery et al., (1996) Br. J. Pharmacol. 119 1491-1497) against 7-OH-DPAT-induced emesis in the ferret.

Antagonist affinity estimates (Table 1) were determined from inhibition of binding of [³H]-spiperone to human recombinant D2short (hD2s) receptors and [³H]-R(+)-7-OH-DPAT to human recombinant D3 (hD3) receptors, using standard techniques.

TABLE 1
Affinity estimates (pKᵢ ± s.e.m.) at hD2s and hD3 receptors (n = 3-11):

| | (S)-eticlopride | L-741,626 | GR218231 |
|---|---|---|---|
| hD2s | 9.3 ± 0.20 | 8.1 ± 0.50 | 6.8 ± 0.44 |
| hD3 | 9.5 ± 0.24 | 6.6 ± 0.24 | 8.7 ± 0.13 |

GR218231 was ˜80-fold selective for hD3 over hD2s receptors, whilst L-741,626 was ˜30 fold selective for hD2s over hD3. Eticlopride was non-selective.

In emesis experiments, ferrets (albino and polecat) of either sex (0.6-1.5 kg) were dosed subcutaneously (s.c.) with antagonist or vehicle 15-45 min prior to challenge with 7-OH-DPAT (0.1 mg/kg s.c.). They were observed for 6 h and emesis quantified as total retches and vomits. All control animals dosed with 7-OH-DPAT exhibited an emetic response (mean±s.e.m.; 78.0±9.0). Anti-emetic potency was expressed as the minimum dose required to completely inhibit emesis in all animals tested (ED₁₀₀; Table 2). Unbound plasma and whole brain concentrations of each antagonist were determined in anaesthetised ferrets (urethane 50% w/v; 3 ml/kg, i.p.), following s.c. dosing at the ED₁₀₀ dose.

TABLE 2
Antagonist potency (ED₁₀₀) versus 7-OH-DPAT-induced emesis
(n = 3), ratio of unbound plasma concentration (Plu, nM) to binding
affinity (Plu/Ki) and brain concentration of each antagonist (n = 1):

| | | |
|---|---|---|
| ED₁₀₀ | Plu/Ki | Brain concⁿ |

| | (mg/kg) | hD3 | hD2s | (ng/g) |
|---|---|---|---|---|
| (S)-eticlopride | 0.03 | 6.8 | 4.4 | 377 |
| L-741,626 | 3 | 0.13 | 4.1 | 250 |
| GR218231 | >10 | 95 | 1.3 | 1670 |

Anti-emetic effects of GR218231 were submaximal even at the highest dose of 10 mg/kg, although the plasma concentration of free drug was −100 times the Ki at hD3 receptors and the brain concentration was the highest attained. Emesis was blocked in only ⅔ animals. Eticlopride and L-741,626 blocked emesis in all animals at free plasma concentrations that were low multiples (˜4×) of the Ki at hD2s. Our data suggests that the emetogenic activity of 7-OH-DPAT, is unrelated to D3 receptor activation, being more likely linked to agonist activity at D2 receptors.

This data strongly suggests that D2 receptors are involved in the induction emesis. A selective D3 agonist with selectivity over D2 receptors will provide an opportunity to increase the therapeutic ratio (therapeutic window; therapeutic index; therapeutic profile) between efficacy in treatment and side effects.

### Example 48

### Selective Activation of Dopamine D2 Receptors Induces Hypotension and Other Cardiovascular Effects in the Anaesthetised Dog

Male beagles were anaesthetised with a mixture of chloralose and urethane, following pre-medication with piritramide. They were instrumented to measure arterial blood pressure, cardiac output, E.C.G., pudendal artery blood flow (PAF) and intra-cavernosal pressure (ICP). Other parameters such as heart rate and vascular resistance were derived from the primary signals. Following a period of equilibration, a series of control readings were taken and the effects of pelvic nerve stimulation at 4 and 8 Hz, on ICP and PAF were measured. Compounds were administered subcutaneously in the dorsal neck region via an implanted catheter, in the dose range of 0.3-3000 μg(base)/kg. Following each dose, parameters were measured at fixed intervals for 30 min. Pelvic nerve stimulations were repeated 15 min after each dose.

Results were analysed as percent change from the final control values for each dog and mean values for three dogs were used.

Three compounds (apomorphine, pramipexole and trans-[4-[(4-Phenylpiperazin-1-yl)methyl]cyclohexyl]-pyrimidin-2-ylamine) produced a reduction in total vascular resistance (TVR; also known as total peripheral resistance; TPR), but only pramipexole and trans-[4-[(4-Phenylpiperazin-1-yl)methyl]cyclohexyl]-pyrimidin-2-ylamine appeared to cause any clear effect on mean blood pressure. The effects on TPR were rapid in onset, peaking at around 10-15 minutes and were evident at dose levels between 1 and 10 μg/kg s.c. An attempt was made to quantify the effect on TPR, by calculating an ED₃₀ value. Sigmoid curves were plotted for each dog, of dose against peak fall, constrained between 100 and 40 (a fall in TPR of 60% is close to maximum). The geometric mean values are shown in Table 3.

TABLE 3

| | Geometric mean |
|---|---|
| Compound | ED₃₀ (μg/kg s.c.) |
| Apomorphine | 11.7 |
| Pramipexole | 8.9 |
| trans-[4-[(4-Phenylpiperazin-1- | 44.6 |
| yl)methyl]cyclohexyl]-pyrimidin-2-ylamine | |
| R-(−)-3-(4-Propylmorpholin-2-yl)phenol | No effect |
| hydrochloride | |

R-(−)-3-(4-Propylmorpholin-2-yl)phenol hydrochloride—No significant effect on blood pressure or TPR was observed at doses up to 3000 μg/kg during the study. Free plasma concentration of the corresponding samples was 2700 nM which is approximately 270-fold greater than the D3 EC50 for R-(−)-3-(4-Propylmorpholin-2-yl)phenol hydrochloride.

Apomorphine, pramipexole and trans-[4-[(4-Phenylpiperazin-1-yl)methyl]cyclohexyl]-pyrimidin-2-ylamine—produced broadly similar increases in cardiac output and heart rate. The D3 selective agonist, R-(−)-3-(4-Propylmorpholin-2-yl)phenol hydrochloride, had no significant effect on cardiac output and heart rate.

The D2 agonist (trans-[4-[(4-Phenylpiperazin-1-yl)methyl]cyclohexyl]-pyrimidin-2-ylamine), the D3-prefering agonist (pramipexole) and the non-selective dopamine agonist (apomorphine) produced marked haemodynamic changes in the anaesthetised dog, with cardiac output being approximately doubled, at the highest dose levels. There were some qualitative differences on blood pressure and heart rate effects, the importance of which are unclear. The most important parameter for evaluating haemodynamic changes, TPR, was reduced in all cases. An ED₃₀ value was calculated for the compounds. This level represents a significant fall in TPR and would probably cause orthostatic hypotension and syncope. It would appear that pramipexole was equipotent to apomorphine, mirroring pramipexole's reduced potency at D2 receptors. The D3 selective agonist (R-(−)-3-(4-Propylmorpholin-2-yl)phenol hydrochloride) had no significant hemodynamic effects in the anaesthetised dog.

The data suggests that the cardiovascular effects observed are due to a D2-mediated effect based upon the trans-[4-[(4-Phenylpiperazin-1-yl)methyl]cyclohexyl]-pyrimidin-2-ylamine data. Both apomorphine and pramipexole are inconclusive in confirming the possible D2-mediated cardiovascular effects due to their lack of selectivity for D3 over D2 receptors.

This data strongly suggests that D2 receptors are involved in the cardiovascular effects associated with dopamine agonists. A selective D3 agonist with selectivity over D2 receptors will provide an opportunity to increase the therapeutic ratio (therapeutic window; therapeutic index; therapeutic profile) between efficacy in treatment and side effects.

### Example 49

### Use of a Selective D3 Agonist to Enhance Penile Erection and Female Genital Blood Flow

R-(−)-3-(4-Propylmorpholin-2-yl)phenol hydrochloride was prepared and tested in said male and female anaesthetised rabbits (method detailed above) to evaluate its effect on penile intracavernosal pressure and cavernosal blood flow and vaginal and clitoral blood flow, respectively.

The investigations found that the functionally selective D3 agonist, R-(−)-3-(4-Propylmorpholin-2-yl)phenol hydrochloride, when tested in male and female anaesthetised rabbits (method detailed above), beneficially enhanced penile intracavernosal pressure and cavernosal blood flow in male rabbits and vaginal and clitoral blood flow in female rabbits to at least the same extent as non-selective dopamine agonists, such as apomorphine and pramipexole.

### Example 50

### Functional Selectivity of R-(−)-3-(4-Propylmorpholin-2-yl)phenol hydrochloride

The functional selectivity of R-(−)-3-(4-Propylmorpholin-2-yl)phenol hydrochloride for dopamine D3 receptors over dopamine D2 receptors was analysed using the “Functional D3/D2 Agonist Assay” detailed above.

Comparisons were made with the following non-selective dopamine agonists: pramipexole, ropinirole, 7-OH-DPAT, quinelorane, trans-[4-[(4-Phenylpiperazin-1-yl)methyl]cyclohexyl]-pyrimidin-2-ylamine and apomorphine.

R-(−)-3-(4-Propylmorpholin-2-yl)phenol hydrochloride is functionally selective for D3 over D2 receptors, when using the same functional assay, the selectivity being more than 3-times that shown by pramipexole (which is only about 5-fold to about 9-fold selective towards D3 receptors, which effectively (functionally) is equivalent to a balanced agonist). In contrast, apomorphine, 7-OH-DPAT and quinelorane were balanced agonists showing more or less equal activity towards both D2 and D3 receptors and trans-[4-[(4-Phenylpiperazin-1-yl)methyl]cyclohexyl]-pyrimidin-2-ylamine was a D2-preferring agonist (see Table 4).

TABLE 4

| | Functional | | | |
|---|---|---|---|---|
| | Potency EC50* (nM) | | D3 | Dopamine |
| Compound | D2L CHO | D3LGH4 | selectivity | receptor |
| (Agonist): | (human) | C1 (human) | over D2 | preference |
| R-(−)-3-(4- | 4973 | 7.3 | 681 | D3 |
| Propylmorpholin- | | | | selective |
| 2-yl)phenol | | | | |
| hydrochloride | | | | |
| Pramipexole** | 2.75 | 0.56 | 4.9 | Slightly |
| | | | | D3- |
| | | | | preferring - |
| | | | | more or |
| | | | | less |
| | | | | balanced |
| Ropinirole | 16.1 | 8.0 | 2.0 | Balanced |
| Quinelorane | 6.1 | 4.8 | 1.3 | Balanced |
| Apomorphine | 7.2 nM | 3.9 nM | 1.8 | Balanced |
| 7-OH-DPAT | 1.1 | 3.0 | 0.4 | Balanced |
| PNU-95666 | 121 | 172 | 0.7 | Balanced |
| Trans-[4-[(4- | 3.8 | No effect | | D2- |
| Phenylpiperazin-1- | | | | selective |
| yl)methyl]cyclohex | | | | |
| yl]-pyrimidin-2- | | | | |
| ylamine | | | | |

*EC50 = “effective concentration 50%” - also written EC₅₀ - and defined, in this context, as “the molar concentration of an agonist which produces 50% (or half) of the maximum possible response for that agonist”.
**Published data on pramipexole is in agreement with this data - see “Pramipexole binding and activation of cloned and expressed dopamine D2, D3 and D4 receptors”, Mierau J, et al. Eur. J. Pharmacol. 1995; 290(1): 29-36, where in the abstract it is stated that “ . . . These results indicate a 5-fold selectivity of pramipexole for D3 receptors, while quinpirole and bromocriptine are non-selective or more D2/D4 receptor selective. . . . ”.

WO 93/23035 discloses ropinirole and suggests that it is a selective D3 agonist and indicates a selectivity based on binding D2:D3 of 1380 nM:69 nM (20-fold). However, such binding data is not an accurate reflection of “functional selectivity” as the data in Table 4 above clearly shows ropinirole not to be D3 selective functionally.

### Example 51

### Comparative Effect of Compounds Having Functionally at Least 30-Fold Selectivity Towards D3 Agonists on One or More Side Effects, Such as Nausea, Vomiting, Hypotension or Syncope

The functionally selective D3 agonist, R-(−)-3-(4-Propylmorpholin-2-yl)phenol hydrochloride, beneficially reduced one or more side effects, such as nausea, vomiting, hypotension or syncope, as compared with the side effects observed when non-selective dopamine agonists, such as apomorphine and pramipexole, were administered.

R-(−)-3-(4-Propylmorpholin-2-yl)phenol hydrochloride (10 μg/kg s.c.), a selective D3 agonist, produced 2.25±0.25 erections/10 minutes in the telemetry rat.

There was no significant effect of R-(−)-3-(4-Propylmorpholin-2-yl)phenol hydrochloride on blood pressure or TPR observed at doses up to 3000 μg/kg during the study. Free plasma concentration of the corresponding samples was 2700 nM which is approximately 270-fold greater than the D3 EC50 for R-(−)-3-(4-Propylmorpholin-2-yl)phenol hydrochloride.

### Example 52

### Comparative Effect of Compounds Having Functionally at Least 30-Fold Selectivity for D3 Receptor Over D2 Receptor on Erection Versus One or More Side Effects, Such as Nausea, Vomiting, Hypotension or Syncope

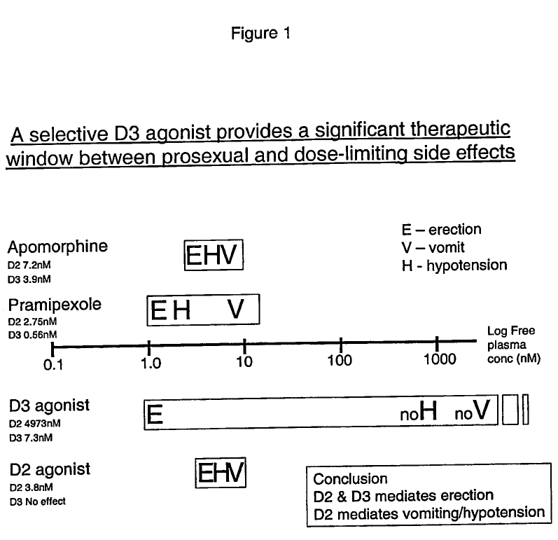

Apomorphine (a non-selective dopamine agonist), pramipexole (a D3-prefering D2/D3 agonist), a selective D3 agonist [R-(−)-3-(4-Propylmorpholin-2-yl)phenol hydrochloride], and a selective D2 agonist [trans-[4-[(4-Phenylpiperazin-1-yl)methyl]cyclohexyl]-pyrimidin-2-ylamine] were profiled in the rat telemetry model of erection, and dog models of nausea/vomiting and hypotension (see FIG. 1).

Only the selective D3 agonist displayed a significant therapeutic index (therapeutic ratio; therapeutic window; therapeutic profile) between beneficial prosexual effects and potential adverse effects of hypotension and nausea/vomiting. The prosexual effects were observed at free plasma concentrations of circa 1 nM and nausea/vomiting and hypotension was absent even at the highest doses tested (circa 1000 nM free drug concentration). Conversely, apomorphine, pramipexole and the selective D2 agonist all induced prosexual effects at similar concentration to those that induced nausea/vomiting and hypotension.

The data generated in these studies demonstrate that D2 and D3 receptors mediate the prosexual effects of dopamine agonists and that D2 receptors mediate the adverse effects of vomiting and hypotension associated with non-selective dopamine agonists. Selectively targeting prosexual D3 dopamine receptors, whilst achieving selectivity over D2 receptors, will improve the therapeutic profile (therapeutic ratio; therapeutic window; therapeutic index) by reducing dose-limiting side effects mediated by other dopamine receptor subtypes e.g. D2.

### Example 53

### A Selective D3 Agonist has No Significant Effects, in Contrast to a D3-Preferring D2/D3 Agonist, on Hemodynamic Parameters in the Anaesthetised Dog

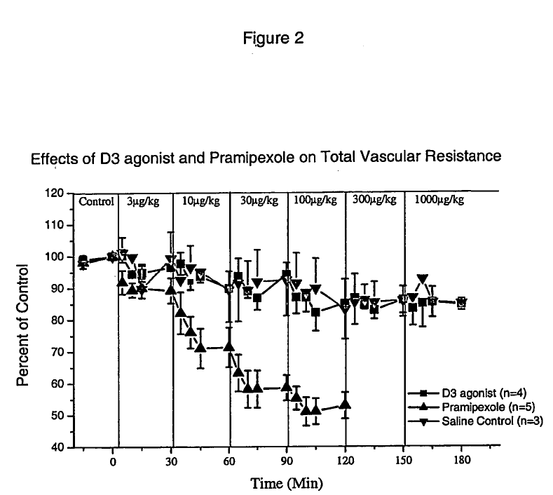

Pramipexole (a D3-prefering D2/D3 agonist) and a selective D3 agonist [R-(−)-3-(4-Propylmorpholin-2-yl)phenol hydrochloride] were profiled in the anaesthetised dog hemodynamic model (see FIG. 2).

Pramipexole produced hypotension—a reduction in total vascular resistance (TPR) and clear effect on mean blood pressure. The effects on TPR were rapid in onset, peaking at around 10-15 minutes and were evident at dose levels between 10 μg/kg s.c. Pramipexole also produced increases in cardiac output and heart rate.

The selective D2 agonist (trans-[4-[(4-Phenylpiperazin-1-yl)methyl]cyclohexyl]-pyrimidin-2-ylamine) produced similar hemodynamic effects as pramipexole (data not shown in FIG. 2—see, instead, Example 48). In contrast to pramipexole and the D2 selective agonist, the selective D3 agonist and the saline control had no significant effects on TPR, mean blood pressure, cardiac output or heart rate.

The data generated in this study demonstrates that D2 receptors mediate the adverse hemodynamic effects of hypotension associated with non-selective dopamine agonists. Selectively targeting prosexual D3 dopamine receptors, whilst achieving selectivity over D2 receptors, will improve the therapeutic profile (therapeutic ratio; therapeutic window; therapeutic index) by reducing dose-limiting side effects mediated by other dopamine receptor subtypes e.g. D2-mediated hypotension.

### Example 54

### Conscious Rat Models of Copulatory Behaviour—Measurements of Male and Female Sexual Desire and Arousal

All studies of sexual behaviour must be performed on adult rats (˜12 weeks of age/200 g weight). Sexual behaviour tests are performed under red light during the dark phase of the light/dark cycle starting at 2 h after lights off and videotaped for further analysis (observer presence can unfavourably alter sexual behaviour).

Male Sexual Behaviour

Males (N=8-10) are placed in a clear aquarium (or home cage) for at least 15 min acclimation period. An estrous female is introduced into the mating arena. The following information is recorded during the 45 min test: latency to begin mounting behaviour, latency to first intromission, latency to first ejaculation (calculated from first intromission), number of intromissions to ejaculation, number of mounts until ejaculation, and the number of ejaculations during the test period. Males are observed during a single 45 min test with an estrous WT female.

The rats are presented with a receptive female at 4 day intervals, i.e. every third day (having 2 clear days between presentations), until completing at least 6 days of baseline determination. By then, vigorous behaviour should be expected. Rats that display sluggish copulatory behaviour are used as a model of HSDD.

For all the sexual behaviour tests, the males are placed in an observation arena (50-60 cm diameter for rats). 3 to 4 minutes after placing the male in the arena, a receptive female (ovariectomised, bearing a 7 mm silastic implant of oestradiol benzoate gives the best results without complications) is introduced to the arena and the following parameters noted:

Mount Latency: time (in seconds) taken between introduction of female and first mount. A maximum time of 15 minutes (900 seconds) is allowed, and the test terminated if no mounts are recorded within that time. Mount latency is related to sexual drive/desire.

Intromission Latency: time (in seconds) taken between introduction of female and first intromission. A maximum time of 15 minutes (900 seconds) is allowed, and the test terminated if no intromissions are recorded within that time. Intromission latency is related to sexual drive/desire.

Number of Mounts: to reach ejaculation. When ejaculation is not reached, the number of mounts is not analysed.

Number of Intromissions: to reach ejaculation. When ejaculation is not reached, the number of intromissions is not analysed.

Copulatory effectsiveness:—no of intromissions/(no of mounts+intromissions). This acts as a measure of erectile function.

Ejaculation Latency: time (in seconds) taken from first intromission to ejaculation. A maximum time of 30 minutes (1800 seconds) is given, and the test terminated if ejaculation is not achieved in that time.

Refractory Period: time (in seconds) taken from ejaculation to the first mount of the next series of sexual activity. In those animals reaching ejaculation the test is terminated at the end of the refractory period, as indicated by the first mount of the next sexual cycle.

These parameters are adjusted for a single ejaculation. If several ejaculations are to be tested, a maximum time or number of ejaculations will need to be included.

A mount is defined as the male assuming the copulatory position, but failing to achieve intromission. Intromission is defined as the males' penis entering the vagina in association with thrusting behaviour. Intromissions are behaviourally defined as the male mounting the female, associated with slow, rhythmic thrusting behaviour. In rats, mounts may be distinguished from intromission because the thrusting behaviour associated with mounting (without intromission) is qualitatively distinct from thrusting behaviour associated with intromissions. Ejaculation is behaviourally defined by the culmination of vigorous thrusting behaviour and the male's arching his spine and lifting his forepaws off the female prior to withdrawal. Ejaculation is verified by the presence of a sperm plug followed by a refractory period of >5 min before the next mounting.

Estrus females are prepared using standard protocols e.g. one day prior to the test, stimulus females receive an s.c. injection of estradiol-17 (0.05 mg suspended in 0.05 cc of sesame seed oil). 6 hours prior to tests, the females are injected s.c. with 0.1 mg progesterone (suspended in 0.05 cc sesame seed oil) to induce behavioural estrus.

An additional male sexual behavioural paradigm is the analysis of non-contact erections as a measure of arousal. Here, a partitioned observation arena is used where the estrus female is placed such that the male erectile response to olfactory stimuli can be recorded. This test can be performed with the same group of rats.

Female Sexual Behaviour

(1) Proceptivity (Sexual Drive/Desire)

The test, in the rat, is carried out in a circular arena of 90 cm diameter surrounded by a 30 cm high wall (these dimensions would need to be adapted for mice). Two small cages with wire-mesh front (15×15 cm) are fixed into the wall such that the front of the cage is “flush” with the wall and the 2 cages are opposite each other. They contain two stimuli animals, a sexually experienced male and a receptive female. The receptive female can either be ovariectomised primed with 5 mg oestradiol benzoate 48 h before the test and 0.5 mg progesterone 4 h before the test or an intact female in oestrous; it is also possible to use ovariectomised females bearing a silastic implant of oestradiol benzoate (7 mm long in the rat). Animals are adapted to the apparatus (in the absence of stimuli animals) for 10 min on 2 consecutive days prior testing. During the 10 min test, time spent investigating each stimulus animal is taken. The difference in the percentage of time spent investigating male minus female is calculated, out of the total time spent investigating stimuli animals. The arena is thoroughly cleaned between animals. The position of the male/female stimuli boxes is randomised between animals, in order to avoid place preference. Sexually naïve animals are used in this test. It would be possible to carry out this test first, followed by receptivity. Female rats would then be used intact and tested in late proestrous, as determined by vaginal smears.

Expected results: In the rat, approximately similar time is spent investigating male or female when the experimental female is in dioestrous or ovariectomised without hormonal priming or primed with low doses of oestrogen. This will result in 0-15% difference.

Females in late proestrous or ovariectomised primed with oestrogen+progesterone spend about 70-80% of the time spent investigating the male and about 20% investigating the female, thus giving a 50-60% difference. Ovariectomised rat primed with oestrogen act as a good model for hypoactive sexual desire disorder where the time spent investigating the opposite sex is directly correlated to sexual drive/desire.

(2) Receptivity

The females (N=8-10) are placed with one or a series of vigorous male rats and subjected to 10 mounts. When the results are not clear it is recommendable to submit them to 20 mounts. The males could be vasectomised or not allowed to ejaculate (by limiting the number of mounts provided by each male). The lordotic response of the animal is recorded and expressed as a percentage of the mounts (i.e. lordosis quotient, LQ).

Ovariectomised females or females in dioestrous will typically not respond to the mounts with lordosis (LQ=0-20%). Animals showing high LQ=80-100% are considered receptive.

Female responses to male mounts or intromissions are scored as either (a) totally unreceptive with kicking, rearing or fleeing (score 0), (b) proceptive/still posture without any extension of legs (score 0.1), (c) proceptive/still posture with extension of legs (score 0.5), or (d) receptive lordosis posture with dorsiflexion of the vertebral column (scores 1-3 with 0.5 intervals, depending on the degree of dorsiflexion). Female responses with the score of 1 or higher are considered as lordosis response for the calculation of lordosis quotient (number of lordosis/number of mounts and intromissions). The percent of proceptive/still postures (score 0.1 and 0.5) among total numbers of mounts and intromissions are calculated separately for each female rat.

R-(−)-3-(4-Propylmorpholin-2-yl)phenol hydrochloride was prepared and tested in said male and female conscious rats (method detailed above) to evaluate its effect on male and female copulatory behaviours, respectively.

Investigations suggest that R-(−)-3-(4-Propylmorpholin-2-yl)phenol hydrochloride beneficially enhances proceptive and receptive behaviours in female rats, and restores sexual desire in oestrogen primed ovarectomised animals (model of HSDD). In male rats, R-(−)-3-(4-Propylmorpholin-2-yl)phenol hydrochloride beneficially enhances copulatory behaviour and copulatory effectiveness (no of intromission/(no of mounts+intromissions)) and reduces the mount latency and intromission latency in sluggish males (model of HSDD).

### Example 55

### Functional Selectivity Data

Using the cAMP enzymeimmunoassay described supra, the EXAMPLE compounds (see “Chemistry Examples” section above) were tested for functional selectivity for a D3 receptor compared to a D2 receptor. All non-intermediate compounds were at least about 15-times more functionally selective for a dopamine D3 receptor as compared with a dopamine D2 receptor when measured using the same functional assay. A selection of the functional selectivity data is given below in Table 5.

TABLE 5

| | Functional | Functional | |
|---|---|---|---|
| | Potency at D3 | Potency at D2 | Functional |
| Compound of | receptor | receptor | selectivity |
| EXAMPLE . . . | (EC50 in nM) | (EC50 in nM) | (D3 over D2) |
| 8 | 7.3 | 4973 | 681-fold |
| 15a | 77 | 1750 | 22.7-fold |
| 15a + 15b | 89 | >10000 | >112-fold |
| (racemate) | | | |
| 23a | 65 | >5000 | >76.9-fold |
| 23a + 23b | ˜65 | >5000 | >76.9-fold |
| (racemate) | | | |
| 35 | 501 | ˜10500 | ˜21.0-fold |
| 37 | 895 | >100000 | >111.7-fold |

EC50 = “effective concentration 50%” - also written EC₅₀ - and defined, in this context, as “the molar concentration of an agonist which produces 50% (or half) of the maximum possible response for that agonist”.

### Example 56

### Use of a S32504 to Enhance Penile Erection and Female Genital Blood Flow

S32504 was prepared and tested in said male and female anaesthetised rabbits (method detailed above) to evaluate its effect on intracavernosal pressure and cavernosal blood flow and vaginal and clitoral blood flow, respectively.

Early investigations suggest that S32504 beneficially enhances intracavernosal pressure and cavernosal blood flow in male rabbits and vaginal and clitoral blood flow in female rabbits.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

By cross-reference herein to compounds contained in patents and patent applications, which can be used in accordance with invention, we mean the therapeutically active compounds as defined in the claims and the specific examples (all of which are incorporated herein by reference).

List of Sequences (Which Forms Part of the Description)

SEQ ID NO: 1
atggcatctctgagtcagctgagtagccacctgaactacacctgtggggcagagaact
ccacaggtgccagccaggcccgcccacatgcctactatgccctctcctactgcgcgct
catcctggccatcgtcttcggcaatggcctggtgtgcatggctgtgctgaaggagcgg
gccctgcagactaccaccaactacttagtagtgagcctggctgtggcagacttgctgg
tggccaccttggtgatgccctgggtggtatacctggaggtgacaggtggagtctggaa
tttcagccgcatttgctgtgatgtttttgtcaccctggatgtcatgatgtgtacagcc
agcatccttaatctctgtgccatcagcatagacaggtacactgcagtggtcatgcccg
ttcactaccagcatggcacgggacagagctcctgtcggcgcgtggccctcatgatcac
ggccgtctgggtactggcctttgctgtgtcctgccctcttctgtttggctttaatacc
acaggggaccccactgtctgctccatctccaaccctgattttgtcatctactcttcag
tggtgtccttctacctgccctttggagtgactgtccttgtctatgccagaatctatgt
ggtgctgaaacaaaggagacggaaaaggatcctcactcgacagaacagtcagtgcaac
agtgtcaggcctggcttcccccaacaaaccctctctcctgacccggcacatctggagc
tgaagcgttactacagcatctgccaggacactgccttgggtggaccaggcttccaaga
aagaggaggagagttgaaaagagaggagaagactcggaattccctgagtcccaccata
gcgcccaagctcagcttagaagttcgaaaactcagcaatggcagattatcgacatctt
tgaagctggggcccctgcaacctcggggagtgccacttcgggagaagaaggcaaccca
aatggtggccattgtgcttggggccttcattgtctgctggctgcccttcttcttgacc
catgttctcaatacccactgccagacatgccacgtgtccccagagctttacagtgcca
cgacatggctgggctacgtgaatagcgccctcaaccctgtgatctataccaccttcaa
tatcgagttccggaaagccttcctcaagatcctgtcttgctga
SEQ ID NO: 2
MASLSQLSSHLNYTCGAENSTGASQARPHAYYALSYCALILAIVFGNGLVCMAVLKER
ALQTTTNYLVVSLAVADLLVATLVMPWVVYLEVTGGVWNFSRICCDVFVTLDVMMCTA
SILNLCAISIDRYTAVVMPVHYQHGTGQSSCRRVALMITAVWVLAFAVSCPLLFGFNT
TGDPTVCSISNPDFVIYSSVVSFYLPFGVTVLVYARIYVVLKQRRRKRILTRQNSQCN
SVRPGFPQQTLSPDPAHLELKRYYSICQDTALGGPGFQERGGELKREEKTRNSLSPTI
APKLSLEVRKLSNGRLSTSLKLGPLQPRGVPLREKKATQMVAIVLGAFIVCWLPFFLT
HVLNTHCQTCHVSPELYSATTWLGYVNSALNPVIYTTFNIEFRKAFLKILSC
SEQ ID NO: 3
GGCACCAGCTCAGCCCCAAGCCACTGCTCTCCCATCCCAGTCCCTGGAAATCCACCCAC
TTGGCCCAGCTCACCCCAACTCCAACCCACTGGGACCCAGTCTCCAGGGGCCTGACTGT
GGGCGGCAGCCACTCCTGAGTGAGCAAAGGTTCCTCCGCGGTGCTCTCCCGTCCAGAG
CCCTGCTGATGGGGAAGTCCGAAGGCCCCGTGGGGATGGTGGAGAGCGCTGGCCGTGC
AGGGCAGAAGCGCCCGGGGTTCCTGGAGGGGGGGCTGCTGCTGCTGCTGCTGCTGGT
GACCGCTGCCCTGGTGGCCTTGGGTGTCCTCTACGCCGACCGCAGAGGGAAGCAGCTG
CCACGCCTTGCTAGCCGGCTGTGCTTCTTACAGGAGGAGAGGACCTTTGTAAAACGAAAA
CCCCGAGGGATCCCAGAGGCCCAAGAGGTGAGCGAGGTCTGCACCACCCCTGGCTGCG
TGATAGCAGCTGCCAGGATCCTCCAGAACATGGACCCGACCACGGAACCGTGTGACGAC
TTCTACCAGTTTGCATGCGGAGGCTGGCTGCGGCGCCACGTGATCCCTGAGACCAACTC
AAGATACAGCATCTTTGACGTCCTCCGCGACGAGCTGGAGGTCATCCTCAAAGCGGTGC
TGGAGAATTCGACTGCCAAGGACCGGCCGGCTGTGGAGAAGGCCAGGACGCTGTACCG
CTCCTGCATGAACCAGAGTGTGATAGAGAAGCGAGGCTCTCAGCCCCTGCTGGACATCT
TGGAGGTGGTGGGAGGCTGGCCGGTGGCGATGGACAGGTGGAACGAGACCGTAGGACT
CGAGTGGGAGCTGGAGCGGCAGCTGGCGCTGATGAACTCACAGTTCAACAGGCGCGTC
CTCATCGACCTCTTCATCTGGAACGACGACCAGAACTCCAGCCGGCACATCATCTACATA
GACCAGCCCACCTTGGGCATGCCCTCCCGAGAGTACTACTTCAACGGCGGCAGCAACCG
GAAGGTGCGGGAAGCCTACCTGCAGTTCATGGTGTCAGTGGCCACGTTGCTGCGGGAG
GATGCAAACCTGCCCAGGGACAGCTGCCTGGTGCAGGAGGACATGGTGCAGGTGCTGG
AGCTGGAGACACAGCTGGCCAAGGCCACGGTACCCCAGGAGGAGAGACACGACGTCAT
CGCCTTGTACCACCGGATGGGACTGGAGGAGCTGCAAAGCCAGTTTGGCCTGAAGGGAT
TTAACTGGACTCTGTTCATACAAACTGTGCTATCCTCTGTCAAAATCAAGCTGCTGCCAGA
TGAGGAAGTGGTGGTCTATGGCATCCCCTACCTGCAGAACCTTGAAAACATCATCGACAC
CTACTCAGCCAGGACCATACAGAACTACCTGGTCTGGCGCCTGGTGCTGGACCGCATTG
GTAGCCTAAGCCAGAGATTCAAGGACACACGAGTGAACTACCGCAAGGCGCTGTTTGGC
ACAATGGTGGAGGAGGTGCGCTGGCGTGAATGTGTGGGCTACGTCAACAGCAACATGGA
GAACGCCGTGGGCTCCCTCTACGTCAGGGAGGCGTTCCCTGGAGACAGCAAGAGCATG
GTCAGAGAACTCATTGACAAGGTGCGGACAGTGTTTGTGGAGACGCTGGACGAGCTGGG
CTGGATGGACGAGGAGTCCAAGAAGAAGGCGCAGGAGAAGGCCATGAGCATCCGGGAG
CAGATCGGGCACCCTGACTACATCCTGGAGGAGATGAACAGGCGCCTGGACGAGGAGT
ACTCCAATCTGAACTTCTCAGAGGACCTGTACTTTGAGAACAGTCTGCAGAACCTCAAGG
TGGGCGCCCAGCGGAGCCTCAGGAAGCTTCGGGAAAAGGTGGACCCAAATCTCTGGAT
CATCGGGGCGGCGGTGGTCAATGCGTTCTACTCCCCAAACCGAAACCAGATTGTATTCC
CTGCCGGGATCCTCCAGCCCCCCTTCTTCAGCAAGGAGCAGCCACAGGCCTTGAACTTT
GGAGGCATTGGGATGGTGATCGGGCACGAGATCACGCACGGCTTTGACGACAATGGCC
GGAACTTCGACAAGAATGGCAACATGATGGATTGGTGGAGTAACTTCTCCACCCAGCACT
TCCGGGAGCAGTCAGAGTGCATGATCTACCAGTACGGCAACTACTCCTGGGACCTGGCA
GACGAACAGAACGTGAACGGATTCAACACCCTTGGGGAAAACATTGCTGACAACGGAGG
GGTGCGGCAAGCCTATAAGGCCTACCTCAAGTGGATGGCAGAGGGTGGCAAGGACCAG
CAGCTGCCCGGCCTGGATCTCACCCATGAGCAGCTCTTCTTCATCAACTATGCCCAGGTG
TGGTGCGGGTCCTACCGGCCCGAGTTCGCCATCCAATCCATCAAGACAGACGTCCACAG
TCCCCTGAAGTACAGGGTACTGGGGTCGCTGCAGAACCTGGCCGCCTTCGCAGACACGT
TCCACTGTGCCCGGGGCACCCCCATGCACCCCAAGGAGCGATGCCGCGTGTGGTAGCC
AAGGCCCTGCCGCGCTGTGCGGCCCACGCCCACCTGCTGCTCGGAGGCATCTGTGCGA
AGGTGCAGCTAGCGGCGACCCAGTGTACGTCCCGCCCCGGCCAACCATGCCAAGCCTG
CCTGCCAGGCCTCTGCGCCTGGCCTAGGGTGCAGCCACCTGCCTGACACCCAGGGATG
AGCAGTGTCCAGTGCAGTACCTGGACCGGAGCCCCCTCCACAGACACCCGCGGGGCTC
AGTGCCCCCGTCACAGCTCTGTAGAGACAATCAACTGTGTCCTGCCCACCCTCCAAGGT
GCATTGTCTTCCAGTATCTACAGCTTCAGACTTGAGCTAAGTAAATGCTTCAAAGAAAAAA
AAAAAAAAAAAAAAA
SEQ ID NO: 4
CAGAGCTCGTTTAGTGAACCGTCAGAATTTTGTAATACGACTCACTATAGGGCGGCCGCG
AATTCGGCACCAGCTCAGCCCGAAGCCACTGCTCTCCCATCCCAGTCCCTGGAAATCCA
CCCACTTGGCCCAGCTCACCCCAACTCCAACCCACTGGGACCCAGTCTCCAGGGGCCTG
ACTGTGGGCGGCAGCCACTCCTGAGTGAGCAAAGGTTCCTCCGCGGTGCTCTCCCGTCC
AGAGCCCTGCTGATGGGGAAGTCCGAAGGCCCCGTGGGGATGGTGGAGAGCGCTGGCC
GTGCAGGGCAGAAGCGCCCGGGGTTCCTGGAGGGGGGGCTGCTGCTGCTGCTGCTGCT
GGTGACCGCTGCCCTGGTGGCCTTGGGTGTCCTCTACGCCGACCGCAGAGGGAAGCAG
CTGCCACGCCTTGCTAGCCGGCTGTGCTTCTTACAGGAGGAGAGGACCTTTGTAAAACG
AAAACCCCGAGGGATCCCAGAGGCCCAAGAGGTGAGCGAGGTCTGCACCACCCCTGGC
TGCGTGATAGCAGCTGCCAGGATCCTCCAGAACATGGACCCGACCACGGAACCGTGTGA
CGACTTCTACCAGTTTGCATGCGGAGGCTGGCTGCGGCGCCACGTGATCCCTGAGACCA
ACTCAAGATACAGCATCTTTGACGTCCTCCGCGACGAGCTGGAGGTCATCCTCAAAGCG
GTGCTGGAGAATTCGACTGCCAAGGACCGGCCGGCTGTGGAGAAGGCCAGGACGCTGT
ACCGCTCCTGCATGAACCAGAGTGTGATAGAGAAGCGAGGCTCTCAGCCCCTGCTGGAC
ATCTTGGAGGTGGTGGGAGGCTGGCCGGTGGCGATGGACAGGTGGAACGAGACCGTAG
GACTCGAGTGGGAGCTGGAGCGGCAGCTGGCGCTGATGAACTCACAGTTCAACAGGCG
CGTCCTCATCGACCTCTTCATCTGGAACGACGACCAGAACTCCAGCCGGCACATCATCTA
CATAGACCAGCCCACCTTGGGCATGCCCTCCCGAGAGTACTACTTCAACGGCGGCAGCA
ACCGGAAGGTGCGGGAAGCCTACCTGCAGTTCATGGTGTCAGTGGCCACGTTGCTGCG
GGAGGATGCAAACCTGCCCAGGGACAGCTGCCTGGTGCAGGAGGACATGGTGCAGGTG
CTGGAGCTGGAGACACAGCTGGCCAAGGCCACGGTACCCCAGGAGGAGAGACACGACG
TCATCGCCTTGTACCACCGGATGGGACTGGAGGAGCTGCAAAGCCAGTTTGGCCTGAAG
GGATTTAACTGGACTCTGTTCATACAAACTGTGCTATCCTCTGTGAAAATCAAGCTGCTGC
CAGATGAGGAAGTGGTGGTCTATGGCATCCCCTACCTGCAGAACCTTGAAAACATCATCG
ACACCTACTCAGCCAGGACCATACAGAACTACCTGGTCTGGCGCCTGGTGCTGGACCGC
ATTGGTAGCCTAAGCCAGAGATTCAAGGACACACGAGTGAACTACCGCAAGGCGCTGTTT
GGCACAATGGTGGAGGAGGTGCGCTGGCGTGAATGTGTGGGCTACGTCAACAGCAACAT
GGAGAACGCCGTGGGCTCCCTCTACGTCAGGGAGGCGTTCCCTGGAGACAGCAAGAGC
ATGGTCAGAGAACTCATTGACAAGGTGCGGACAGTGTTTGTGGAGACGCTGGACGAGCT
GGGCTGGATGGACGAGGAGTCCAAGAAGAAGGCGCAGGAGAAGGCCATGAGCATCCGG
GAGCAGATCGGGCACCCTGACTACATCCTGGAGGAGATGAACAGGCGCCTGGACGAGG
AGTACTCCAATCTGAACTTCTCAGAGGACCTGTACTTTGAGAACAGTCTGCAGAACCTCA
AGGTGGGCGCCCAGCGGAGCCTCAGGAAGCTTCGGGAAAAGGTGGACCCAAATCTCTG
GATCATCGGGGCGGCGGTGGTCAATGCGTTCTACTCCCCAAACCGAAACCAGATTGTATT
CCCTGCCGGGATCCTCCAGCCCCCCTTCTTCAGCAAGGAGCAGCCACAGGCCTTGAACT
TTGGAGGCATTGGGATGGTGATCGGGCACGAGATCACGCACGGCTTTGACGACAATGGC
CGGAACTTCGACAAGAATGGCAACATGATGGATTGGTGGAGTAACTTCTCCACCCAGCAC
TTCCGGGAGCAGTCAGAGTGCATGATCTACCAGTACGGCAACTACTCCTGGGACCTGGC
AGACGAACAGAACGTGAACGGATTCAACACCCTTGGGGAAAACATTGCTGACAACGGAG
GGGTGCGGCAAGCCTATAAGGCCTACCTCAAGTGGATGGCAGAGGGTGGCAAGGACCA
GCAGCTGCCCGGCCTGGATCTCACCCATGAGCAGCTCTTCTTCATCAACTATGCCCAGGT
GTGGTGCGGGTCCTACCGGCCCGAGTTCGCCATCCAATCCATCAAGACAGACGTCCACA
GTCCCCTGAAGTACAGGGTACTGGGGTCGCTGCAGAACCTGGCCGCCTTCGCAGACAC
GTTCCACTGTGCCCGGGGCACCCCCATGCACCCCAAGGAGCGATGCCGCGTGTGGTAG
CCAAGGCCCTGCCGCGCTGTGCGGCCCACGCCCACCTGCTGCTCGGAGGCATCTGTGC
GAAGGTGCAGCTAGCGGCGACCCAGTGTACGTCCCGCCCCGGCCAACCATGCCAAGCC
TGCCTGCCAGGCCTCTGCGCCTGGCCTAGGGTGCAGCCACCTGCCTGACACCCAGGGA
TGAGCAGTGTCCAGTGCAGTACCTGGACCGGAGCCCCCTCCACAGACACCCGCGGGGC
TCAGTGCCCCCGTCACAGCTCTGTAGAGACAATCAACTGTGTCCTGCCCACCCTCCAAG
GTGCATTGTCTTCCAGTATCTACAGCTTCAGACTTGAGCTAAGTAAATGCTTCAAAGAAA
AAAAAAAAAAAAAAAAACTCGACTCTAGATTGCG
SEQ ID NO: 5
MGKSEGPVGMVESAGRAGQKRPGFLEGGLLLLLLLVTAALVALGVLYADRRGKQLPRLAS
RLCFLQEERTFVKRKPRGIPEAQEVSEVCTTPGCVIAAARILQNMDPTTEPCDDFYQFAC
GGWLRRHVIPETNSRYSIFDVLRDELEVILKAVLENSTAKDRPAVEKARTLYRSCMNQSV
IEKRGSQPLLDILEVVGGWPVAMDRWNETVGLEWELERQLALMNSQFNRRVLIDLFIWND
DQNSSRHIIYIDQPTLGMPSREYYFNGGSNRKVREAYLQFMVSVATLLREDANLPRDSCL
VQEDMVQVLELETQLAKATVPQEERHDVIALYHRMGLEELQSQFGLKGFNWTLFIQTVLS
SVKIKLLPDEEVVVYGIPYLQNLENIIDTYSARTIQNYLVWRLVLDRIGSLSQRFKDTRV
NYRKALFGTMVEEVRWRECVGYVNSNMENAVGSLYVREAFPGDSKSAAVRELIDKVRTVFV
ETLDELGWAADEESKKKAQEKAMSIREQIGHPDYILEEMNRRLDEEYSNLNFSEDLYFENS
LQNLKVGAQRSLRKLREKVDPNLWIIGAAVVNAFYSPNRNQIVFPAGILQPPFFSKEQPQ
ALNFGGIGMVIGHEITHGFDDNGRNFDKNGNMMDWWSNFSTQHFREQSECAAIYQYGNYSW
DLADEQNVNGFNTLGENIADNGGVRQAYKAYLKWMAEGGKDQQLPGLDLTHEQLFFINYA
QVWCGSYRPEFAIQSIKTDVHSPLKYRVLGSLQNLAAFADTFHCARGTPMHPKERCRVW

1. (canceled)

2. (canceled)

3. (canceled)

4. (canceled)

5. (canceled)

6. (canceled)

7. (canceled)

8. A method of treating and/or preventing female sexual dysfunction (FSD), male erectile dysfunction (MED), male anorgasmia, or male hypoactive sexual desire disorder (HSDD) in a human or animal which method comprises administering to an individual an effective amount of a pharmaceutical composition comprising a selective dopamine D3 receptor agonist, wherein said dopamine D3 receptor agonist is at least about 15-times more functionally selective for a dopamine D3 receptor as compared with a dopamine D2 receptor when measured using the same functional assay; and wherein said agonist is optionally admixed with a pharmaceutically acceptable carrier, diluent or excipient.

9. The method according to claim 8, wherein said dopamine D3 receptor agonist is at least about 20-times more functionally selective for a dopamine D3 receptor as compared with a dopamine D2 receptor when measured using the same functional assay.

10. The method according to claim 8, wherein said dopamine D3 receptor agonist is at least about 30-times more functionally selective for a dopamine D3 receptor as compared with a dopamine D2 receptor when measured using the same functional assay.

11. The method according to claim 8, wherein said dopamine D3 receptor agonist is at least about 50-times more functionally selective for a dopamine D3 receptor as compared with a dopamine D2 receptor when measured using the same functional assay.

12. The method according to any one of claims 8 to 11, wherein said FSD is (a) female sexual arousal disorder (FSAD) and/or female HSDD, or (b) female anorgasmia.

13. An assay method for identifying an agent (hereinafter referred to as a selective dopamine D3 agonist) that can be used to treat and/or prevent female sexual dysfunction (FSD), male erectile dysfunction (MED), male anorgasmia, or male hypoactive sexual desire disorder (HSDD), the assay comprising: determining whether a test agent can directly enhance the endogenous genital engorgement process or erectile process; wherein said enhancement is defined as a potentiation of genital blood flow or intracavernosal pressure and/or cavernosal blood flow in the presence of a test agent as defined herein; such potentiation by a test agent is indicative that the test agent may be useful in the treatment and/or prevention of female sexual dysfunction (FSD), male erectile dysfunction (MED), male anorgasmia, or male hypoactive sexual desire disorder (HSDD), and wherein said test agent is a selective dopamine D3 receptor agonist.

14. A diagnostic method, the method comprising isolating a sample from a female or male; determining whether the sample contains an entity present in such an amount as to cause female sexual dysfunction or male sexual dysfunction; wherein the entity has a direct effect on the endogenous genital arousal process in the female or erectile process in the corpus cavernosum of the male; and wherein said entity can be modulated to achieve a beneficial effect by use of an agent; and wherein said agent is a selective dopamine D3 receptor agonist.

15. A diagnostic composition or kit comprising means for detecting an entity in an isolated female or male sample; wherein the means can be used to determine whether the sample contains the entity and in such an amount as to cause female sexual dysfunction (FSD), male erectile dysfunction (MED), male anorgasmia, or male hypoactive sexual desire disorder (HSDD); wherein the entity has a direct effect on the endogenous genital arousal process or erectile process and wherein said entity can be modulated to achieve a beneficial effect by use of an agent; and wherein said agent is a selective dopamine D3 receptor agonist.

16. An animal model used to identify agents capable of treating and/or preventing female sexual dysfunction (FSD), male erectile dysfunction (MED), male anorgasmia, or male hypoactive sexual desire disorder (HSDD), said model comprising an anaesthetised female or male animal including means to measure changes in vaginal/clitoral blood flow, intracavernosal pressure and/or cavernosal blood flow of said animal following stimulation of the pelvic nerve thereof; and wherein said agent in a selective dopamine D3 receptor agonist.

17. An assay method for identifying an agent that can directly enhance the endogenous genital arousal process or erectile process in order to treat FSAD or MED, the assay method comprising: administering an agent to the animal model according to claim 16; and measuring the change in the endogenous genital arousal process or erectile process; wherein said change is defined as a potentiation of vaginal/clitoral blood flow, intracavernosal pressure (ICP) (and/or cavernosal blood flow) in the animal model in the presence of a test agent as defined; and wherein said agent is a selective dopamine D3 receptor agonist.

18. A method according to claims 8-11 further comprising one or more of the following auxiliary agents:
a. A PDE inhibitor (PDEi);
b. α-adrenergic receptor antagonist compounds;
c. An NPY-Y1 antagonist;
d. Cholesterol lowering agents;
e. Estrogen receptor modulators and/or estrogen agonists and/or estrogen antagonists;
f. Androgen receptor modulators and/or androgen agonists and/or androgen antagonists;
g. A testosterone replacement agent or a testosterone implant; or
h. Estrogen, estrogen and medroxyprogesterone or medroxyprogesterone acetate (MPA), alone or as a combination, or estrogen and methyl testosterone hormone replacement therapy agent.

19. (canceled)

20. (canceled)

21. (canceled)

22. The method according to claim 18, wherein said FSD is (a) female sexual arousal disorder (FSAD) and/or female HSDD, or (b) female anorgasmia.

23. The method according to claims 8-11 wherein the composition is administered by mouth or intranasally.

24. The method according to claim 22, wherein the composition is administered by mouth or intranasally.

25. The method according to claims 8-11, wherein said selective dopamine D3 receptor agonist is administered before and/or during sexual arousal.

26. The method according to claim 22 wherein the dopamine D3 receptor agonist is administered before and/or during sexual arousal.
